(19) United States
(12) Patent Application Publication
(10) Pub. No.: US 2010/0162428 A1
(43) Pub. Date: Jun. 24, 2010

(54) Method Of Excising A Nucleic Acid Sequence From A Plant Genome

(75) Inventors: Jeffrey A. Brown, Apex, NC (US); Fang-Ming Lai, Cary, NC (US); Christina E. Roche, Youngsville, NC (US); Hee-Sook Song, Raleigh, NC (US)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/600,902

(22) PCT Filed: May 30, 2008
(86) PCT No.: PCT/EP2008/056691

§ 371 Date: Nov. 19, 2009

Related U.S. Application Data
(60) Provisional application No. 60/941,227, filed on May 31, 2007.

(51) Int. Cl. A01H 1/00; A01H 5/00

(52) U.S. Cl. 800/270; 800/298

(57) ABSTRACT

The present invention relates to a method for excising a nucleic acid sequence from the genome of a plant or a plant cell. This method is based on the steps of transforming a plant cell with a construct encoding a DNA double strand break inducing enzyme (DSBI), generating a transgenic plant line, performing a transient assay to analyze the functionality of the transgenic enzyme, crossing the plant line with a line containing a nucleic acid sequence to be excised and performing an immature embryo conversion or a tissue culture regeneration through callus formation. The method can also be reversed, which means that a plant cell is transformed with a construct encoding a nucleic acid sequence to be excised, and the crossing is performed with a plant line containing a DSBI. As an alternative to the crossing step, a re-transformation of a transgenic plant line with a second construct can also be performed. The invention is also directed to a plant obtained by this method, or progeny, propagation material, part, tissue, cell or cell culture, derived from such a plant. Finally, the invention relates to the use of a plant or progeny, propagation material, part, tissue, cell or cell culture, derived from this method, as aliment, fodder or seeds or for the production of pharmaceuticals or chemicals.

### SUMMARY OF THE INVENTION

The present invention relates to a method for excising a nucleic acid sequence from the genome of a plant or a plant cell. This method is based on the steps of transforming a plant cell with a construct encoding a DNA double strand break inducing enzyme (DSBI), generating a transgenic plant line, performing a transient assay to analyze the functionality of the transgenic enzyme, crossing the plant line with a line containing a nucleic acid sequence to be excised and performing an immature embryo conversion or a tissue culture regeneration through callus formation. The method can also be reversed, which means that a plant cell is transformed with a construct encoding a nucleic acid sequence to be excised, and the crossing is performed with a plant line containing a DSBI. As an alternative to the crossing step, a re-transformation of a transgenic plant line with a second construct can also be performed. The invention is also directed to a plant obtained by this method, or progeny, propagation material, part, tissue, cell or cell culture, derived from such a plant. Finally, the invention relates to the use of a plant or progeny, propagation material, part, tissue, cell or cell culture, derived from this method, as aliment, fodder or seeds or for the production of pharmaceuticals or chemicals.

### BACKGROUND ART

An aim of plant biotechnology is the generation of plants with advantageous novel characteristics, for example for increasing agricultural productivity, improving the quality in foodstuffs or for the production of certain chemicals or pharmaceuticals (Dunwell J. M. (2000) J. Exp. Bot. 51: 487-96).

Transgenic plants can be generated by a variety of techniques (Review: Potrykus I. and Spangenberg G. ed. (1995) Gene transfer to plants. Springer, Berlin) that typically involve the introduction of separate trait and selectable marker genes. The trait gene, or gene of interest, provides the desired trait, while the selectable marker gene (such as a herbicide resistance gene) provides a means during the transformation process of selecting plants that contain the introduced DNA. The selectable marker gene typically provides no useful function once the transformed plant has been identified. The persistence of the selectable marker gene contributes substantially to the lack of acceptance of these “gene food” products among consumers. Thus, there is a demand to develop techniques by means of which marker DNA can be excised from the plant genome in a time-saving and efficient way.

In addition to improving public acceptance, removal of selectable markers can increase the ease in which multiple traits are combined into a single plant (trait stacking) by facilitating retransformation with the same selectable marker or allowing multiple traits to be crossed into a single line without resulting in multiple copies of the selectable marker.

The skilled worker is familiar with a variety of systems for the site-directed removal of recombinantly introduced nucleic acid sequences. One such system is based on the use of sequence specific recombinases (double strand break inducing enzymes, or DSBI) and two recognition sequences of said recombinase which flank the region to be removed. The effect of the DSBI on this construct brings about the excision of the flanked sequence with one of the recognition sequences remaining in the genome. Various sequence-specific recombination systems are described, such as the Cre/lox system of the bacteriophage P1 (Dale E C and Ow D W (1991) Proc Natl Acad Sci USA 88:10558-10562; Russell S H et al. (1992) Mol Gen Genet. 234: 49-59; Osborne B I et al. (1995) Plant J. 7, 687-701), the yeast FLP/FRT system (Kilby N J et al. (1995) Plant J 8:637-652; Lyznik L A et al. (1996) Nucleic Acids Res 24:3784-3789), the Mu phage Gin recombinase, the E. coli Pin recombinase or the R/RS system of the plasmid pSR1 (Onouchi H et al. (1995) Mol Gen Genet. 247:653-660; Sugita K et al. (2000) Plant J. 22:461-469).

A disadvantage of the sequence-specific recombination systems is the reversibility of the reaction, that is to say an equilibrium exists between excision and integration of the marker sequence in question. This frequently brings about unwanted mutations by multiple consecutive insertions and excisions. This not only applies to the Cre-lox system, but also to the other sequence-specific recombinases (see above). A further disadvantage is the fact that one of the recognition sequences of the recombinase remains in the genome, which is thus modified: The remaining recognition sequence excludes a further use of the recombination system, for example for a second genetic modification, since interactions with the subsequently introduced recognition sequences cannot be ruled out. Substantial chromosomal rearrangements or deletions may result.

The conventional approach for identifying double strand break (DSB) induced homologous recombination (HR) in transgenic plant lines requires a minimum time of about 19 months. After the transformation of i) a plant line with a vector encoding a DSBI and ii) a plant line with a vector encoding the sequence to be excised, single copy homozygous lines are identified in the T₀ to T₂ generations of both plant lines. Then the two lines are crossed to create an F₁ line, and it is only the F₂ line which is analyzed for DSB induced HR (see FIG. 9).

### DETAILED DESCRIPTION OF THE INVENTION

The inventors demonstrate the development of a novel plant regeneration strategy that may allow for shortening the process of getting DSB-mediated repair plant lines, collecting more data than crossing (in vitro tissue culture materials versus conventional crossing approach), and reducing the greenhouse space and labor (see FIG. 9).

In this context the inventors developed an improved method for excising a nucleic acid sequence from the genome of a plant or a plant cell. The sequence to be excised can be, for example, a marker gene, and the marker gene can be e.g. a selectable marker gene cassette for selection of transgenic plants or the entire T-DNA region in a transgenic plant for trait containment purpose. The invention is directed to data on establishment of DSB mediated repair for marker excision in plants using DSBI enzymes, especially homing endonucleases (HENs). The introduced DSB can preferably be repaired by homologous recombination (HR), nonhomologous end joining (NHEJ), precise ligation (PL), or other mechanism so that the sequence of interest is fully excised.

The invention is therefore directed to a method for excising a nucleic acid sequence from the genome of a plant or of a plant cell, comprising:
a) transforming a plant cell with a construct encoding a DNA double strand break inducing enzyme,
b) generating a transgenic plant line from the cell of step a),
c) performing a transient assay with the plant line of step b) or cells or parts thereof to analyze the functionality of the transgenic DNA double strand break inducing enzyme,
d) crossing the plant line of step b) with a plant line containing a nucleic acid sequence to be excised, wherein the nucleic acid sequence to be excised comprises at least one recognition sequence which is specific for the enzyme of step a) for the site-directed induction of DNA double strand breaks, and wherein the nucleic acid sequence to be excised is bordered at both sides by a repeated sequence which allows for a DNA repair mechanism, and
e) performing an immature embryo conversion or a tissue culture regeneration through callus formation.

Preferably the DNA repair mechanism of step d) is homologous recombination.

The term “DNA double strand break inducing enzyme” (DSBI) generally refers to all those enzymes which are capable of generating double strand breaks in double stranded DNA in a sequence specific manner at one or more recognition sequences or recognition sites. The DNA break or cleavage may result in blunt ends or so called “sticky” ends of the DNA (having a 5′- or 3′-overhang). The cleavage site may be localized within or outside the recognition sequence of the enzyme. The subsequent excision of the nucleic acid sequence from the genome of a plant or plant cell is preferably realized by homologous recombination between the homologous or “repeated” sequences that should be induced by the double strand break. General methods are disclosed for example in WO 03/004659. Various enzymes suitable for the induction of double strand breaks are known in the art. The following DSBIs are mentioned by way of example, but not by limitation:
1. Restriction endonucleases (e.g. type II), preferably homing endonucleases as described in detail herein.
2. Recombinases (such as, for example, Cre/lox; R—RS; FLP/FTR).
3. Transposases, for example the P-element transposase (Kaufman P D and Rio D C (1992) Cell 69(1):27-39) or AcDs (Xiao Y L and Peterson T (2000) Mol Gen Genet. 263(1):22-29). In principle, all transposases or integrases are suitable as long as they have sequence specificity (Haren L et al. (1999) Annu Rev Microbiol. 1999; 53:245-281; Beall E L, Rio D C (1997) Genes Dev. 11(16):2137-2151).
4. Chimeric nucleases as described herein.
5. Enzymes which induce double-strand breaks in the immune system, such as the RAG1/RAG2 system (Agrawal A et al. (1998) Nature 394(6695):744-451).
6. Group II endonucleases or group II intron endonucleases. Modifications of the intron sequence allows group II introns to be directed to virtually any sequence in a double-stranded DNA, where group II introns can subsequently insert by means of a reverse splice mechanism (Mohr et al. (2000) Genes & Development 14:559-573; Guo et al. (2000) Science 289:452-457). During this reverse splice mechanism, a double-strand break is introduced into the target DNA, the excised intron RNA cleaving the sense strand while the protein portion of the group II intron endonuclease hydrolyses the antisense strand (Guo et al. (1997) EMBO J. 16: 6835-6848). If it is only desired to induce the double-strand break without achieving complete reverse splicing, as is preferably the case in the present invention, it is possible to resort to, for example, group II intron endonucleases which lack the reverse transcriptase activity. While this does not prevent the generation of the double-strand break, the reverse splicing mechanism cannot proceed to completion.

Preferably, the DSBI is chosen in a way that its corresponding recognition sequences are rarely, if ever, found in the unmodified genome of the target plant organism. Ideally, the only copy (or copies) of the recognition sequence in the genome is (or are) the one(s) comprised within the nucleic acid to be excised, thereby eliminating the chance that other DNA in the genome is excised or rearranged when the DSBI is expressed.

The term “expression” refers to the biosynthesis of a gene product. For example, in the case of a structural gene, expression involves transcription of the structural gene into mRNA and optionally the subsequent translation of mRNA into one or more polypeptides.

The term “genome” or “genomic DNA” is referring to the heritable genetic information of a host organism. Said genomic DNA comprises the entire genetic material of a cell or an organism, including the DNA of the nucleus (chromosomal DNA), extrachromosomal DNA, and organellar DNA (e.g. of mitochondria and plastids like chloroplasts). Preferably the terms genome or genomic DNA is referring to the chromosomal DNA of the nucleus.

The term “chromosomal DNA” or “chromosomal DNA sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), and in situ PCR.

One criterion for selecting a suitable DSBI is the length of its corresponding recognition sequence. Said recognition sequence has an appropriate length to allow for rare cleavage (or DSB), more preferably cleavage only at the recognition sequence(s) comprised in the DNA construct of the invention. One factor determining the minimum length of said recognition sequence is—from a statistical point of view—the size of the genome of the host plant. In a preferred embodiment the recognition sequence has a length of at least 10 base pairs, preferably at least 14 base pairs, more preferably at least 16 base pairs, especially preferably at least 18 base pairs, most preferably at least 20 base pairs. A DSBI enzyme that cleaves a 10 base pair recognition sequence is described in Huang B. et al. (1996) J Protein Chem 15 (5): 481-9.

Suitable enzymes are not only natural enzymes, but also synthetic enzymes. Preferred enzymes are all those DSBI enzymes whose recognition sequence is known and which can either be obtained in the form of their proteins (for example by purification) or expressed using their nucleic acid sequence. Especially preferred are those enzymes which have no or only a few recognition sequences—besides the recognition sequences present in the nucleic acid to be excised—in the genomic sequence of a particular plant. This avoids further double strand breaks at undesired loci in the genome.

This is why homing endonucleases are very especially preferred (Review: Belfort M. and Roberts R. J. (1997) Nucleic Acids Res 25: 3379-3388; Jasin M. (1996) Trends Genet. 12:224-228; Internet: http://rebase.neb.com/rebase/rebase.homing.html). Owing to their long recognition sequences, they have no, or only a few, further recognition sequences in the genomic DNA of eukaryotic organisms in most cases.

The sequences encoding for homing endonucleases can be isolated for example from the chloroplast genome of Chlamydomonas (Turmel M et al. (1993) J Mol Biol 232: 446-467). They are small (usually 18 to 26 kD) and their open reading frame (ORF) has a “codon usage” which is suitable directly for nuclear expression in eukaryotes (Monnat R. J. Jr et al. (1999) Biochem Biophys Res Com 255:88-93). Homing endonucleases which are especially preferably isolated are the homing endonucleases I-SceI (WO96/14408), I-SceII (Sarguiel B et al. (1990) Nucleic Acids Res 18:5659-5665), I-SceIII (Sarguiel B et al. (1991) Mol Gen Genet. 255:340-341), I-CeuI (Marshall (1991) Gene 104:241-245), I-CreI (Wang J et al. (1997) Nucleic Acids Res 25: 3767-3776), I-ChuI (Cote V et al. (1993) Gen 129:69-76), 1-TevI (Chu et al. (1990) Proc Natl Acad Sci USA 87:3574-3578; Bell-Pedersen et al. (1990) Nucleic Acids Res 18:3763-3770), 1-TevII (Bell-Pedersen et al. (1990) Nucleic Acids Res 18:3763-3770), I-TevIII (Eddy et al. (1991) Genes Dev. 5:1032-1041), Endo SceI (Kawasaki et al. (1991) J Biol Chem 266:5342-5347), I-CpaI (Turmel M et al. (1995a) Nucleic Acids Res 23:2519-2525) and I-CpaII (Turmel M et al. (1995b) Mol. Biol. Evol. 12, 533-545).

Further examples which may be mentioned are homing endonucleases such as F-SceI, F-SceII, F-SuvI, F-TevI, F-TevII, I-AmaI, I-AniI, I-CeuI, I-CeuAIIP, I-ChuI, I-CmoeI, I-CpaI, I-CpaII, I-CreI, I-CrepsbIP, I-CrepsbIIP, I-CrepsbIIIP, I-CrepsbIVP, I-CsmI, I-CvuI, 1-CvuAIP, I-DdiI, I-DdiII, I-DirI, I-DmoI, I-HmuI, I-HmuII, I-HspNIP, I-LlaI, I-MsoI, I-NaaI, I-NanI, I-NclIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PpbIP, I-PpoI, I-SPBetaIP, I-ScaI, I-SceI, I-SceII, I-SceIII, I-SceIV, I-SceV, I-SceVI, I-SceVII, I-SexIP, I-SneIP, I-SpomCP, I-SpomIP, I-SpomIIP, I-SquIP, I-Ssp6803I, I-SthPhiJP, I-SthPhiST3P, I-SthPhiS3bP, I-TdeIP, I-TevI, I-TevII, I-TevIII, I-UarAP, I-UarHGPA1P, I-UarHGPA13P, I-VinIP, I-ZbiIP, PI-MtuI, PI-MtuHIP, PI-MtuHIP, PI-PfuI, PI-PfuII, PI-PkoI, PI-PkoII, PI-PspI, PI-Rma43812IP, PI-SPBetaIP, PI-SceI, PI-TfuI, PI-TfuII, PI-ThyI, PI-TliI, PI-TliII, and combinations thereof.

The enzymes can be isolated from their organisms of origin in the manner with which the skilled worker is familiar, and/or their coding nucleic acid sequence can be cloned. The sequences of various enzymes are deposited in GenBank.

Other suitable DSBI enzymes that may be mentioned by way of example are chimeric nucleases which are composed of an unspecific catalytic nuclease domain and a sequence specific DNA binding domaine consisting of zinc fingers (Bibikova M et al. (2001) Mol Cell Biol. 21:289-297). These DNA-binding zinc finger domains can be adapted to suit any DNA sequence. Suitable methods for preparing suitable zinc finger domains are described and known to the skilled worker (Beerli R. R. et al., Proc. Natl. Acad. Sci. USA. 2000; 97 (4):1495-1500; Beerli R. R. et al., J. Biol. Chem. 2000; 275(42):32617-32627; Segal D. J. and Barbas C. F. 3rd., Curr. Opin. Chem. Biol. 2000; 4(1):34-39; Kang J S and Kim J S, J Biol Chem 2000; 275(12):8742-8748; Beerli R R et al., Proc Natl Acad Sci USA 1998; 95(25):14628-14633; Kim J S et al., Proc Natl Acad Sci USA 1997; 94(8):3616-3620; Klug A, J Mol Biol 1999; 293(2):215-218; Tsai S Y et al., Adv Drug Deliv Rev 1998; 30(1-3):23-31; Mapp A K et al., Proc Natl Acad Sci USA 2000; 97(8):3930-3935; Sharrocks A D et al., Int J Biochem Cell Biol 1997; 29(12):1371-1387; Zhang L et al., J Biol Chem 2000; 275(43):33850-33860).

In some aspects of the invention, molecular evolution can be employed to create an improved DSBI. Polynucleotides encoding a candidate DSBI enzyme can, for example, be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer (1994) Proc Natl Acad Sci USA 91: 10747-10751; Stemmer (1994) Nature 370: 389-391; and U.S. Pat. No. 5,605,793, U.S. Pat. No. 5,837,458, U.S. Pat. No. 5,830,721 and U.S. Pat. No. 5,811,238. An alternative to DNA shuffling for the modification of DSBI is rational design. Rational design involves the directed mutation of a gene based on an existing understanding of DNA and/or protein interactions so that the outcome of the mutation is anticipated.

The DSBI enzyme can also be expressed as a fusion protein with a nuclear localization sequence (NLS). This NLS sequence enables facilitated transport into the nucleus and increases the efficacy of the recombination system. A variety of NLS sequences are known to the skilled worker and described, inter alia, by Jicks G R and Raikhel N V (1995) Annu Rev. Cell Biol. 11:155-188. Preferred for plant organisms is, for example, the NLS sequence of the SV40 large antigen. Owing to the small size of many DSBI enzymes (such as, for example, the homing endonucleases), an NLS sequence is however not necessarily required. These enzymes can be capable of passing through the nuclear pores without the need for transport processes mediated by an NLS.

For the present invention, the DNA double strand break inducing enzyme is preferably selected from the group consisting of homing endonucleases, restriction endonucleases, group II endonucleases, recombinases, transposases and chimeric endonucleases.

More preferably, the DNA double strand break inducing enzyme is selected from the group consisting of I-SceI, F-SceI, F-SceII, F-SuvI, F-TevI, F-TevII, I-AmaI, I-AniI, I-CeuI, I-CeuAIIP, I-ChuI, I-CmoeI, I-CpaI, I-CpaII, I-CreI, I-CrepsbIP, I-CrepsbIIP, I-CrepsbIIIP, I-CrepsbIVP, I-CsmI, I-CvuI, I-CvuAIP, I-DdiI, I-DdiII, I-DirI, I-DmoI, I-HmuI, I-HmuII, I-HspNIP, I-LlaI, I-MsoI, I-NaaI, I-NanI, I-NclIP, I-NgrIP, I-NitI, I-NjaI, I-Nsp236IP, I-Paid, I-PboIP, I-PcuIP, I-PcuAI, I-PcuVI, I-PgrIP, I-PobIP, I-Pod, I-PorIIP, I-PpbIP, I-PpoI, I-SPBetaIP, I-ScaI, I-SceII, I-SceIII, I-SceIV, I-SceV, I-SceVI, I-SceVII, I-SexIP, I-SneIP, I-SpomCP, I-SpomCP, I-SpomIIP, I-SquIP, I-Ssp6803I, I-SthPhiJP, I-SthPhiST3P, I-SthPhiS3bP, I-TdeIP, I-TevI, RTI I-TevII, I-TevIII, I-UarAP, I-UarHGPA1P, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-PspI, PI-Rma43812IP, PI-SPBetaIP, PI-SceI, PI-TfuI, PI-TfuII, PI-ThyI, PI-TliI and PI-TliII.

Most preferably the DNA double strand break inducing enzyme is selected from the group consisting of enzymes having a nucleotide sequence as depicted in SEQ ID NOs: 26 or 27 or a substantial homologue thereof.

As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide”, “peptide”, “oligopeptide”, “polypeptide”, “gene product”, “expression product” and “protein” are used interchangeably herein to refer to a polymer or oligomer of consecutive amino acid residues.

The term “nucleic acid” refers to deoxyribonucleotides or ribonucleotides and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” can represent for example a gene, a cDNA, an mRNA, an oligonucleotide and a polynucleotide.

The phrase “nucleic acid sequence” refers to a single or double-stranded polymer of deoxyribonucleotide or ribonucleotide bases usually read from the 5′- to the 3′-end. It can have any length from only a few nucleotides to many kilo bases and includes chromosomal DNA, self-replicating plasmids, infectious polymers of DNA or RNA and DNA or RNA that performs a primarily structural role.

A “coding region” is the portion of the nucleic acid that is transcribed to an mRNA and directs the translation of the specified protein sequence. Eventually the mRNA is translated in a sequence-specific manner to produce into a particular polypeptide or protein. The coding region is said to encode such a polypeptide or protein. The coding region is bounded, in eukaryotes, on the 5′-side by the nucleotide triplet “ATG” which encodes the initiator methionine and on the 3′-side by one of the three triplets that specify stop codons (i.e., TAA, TAG, TGA). In addition to containing introns, genomic forms of a gene may also include sequences located on both the 5′- and 3′-end of the sequences that are present on the RNA transcript. These sequences are referred to as untranslated regions or UTRs; these UTRs are located 5′ or 3′ to the coding region of the mRNA. The 5′-UTR may contain regulatory sequences such as enhancers that can control or influence the transcription of the gene. The 3′-flanking region may contain sequences that can provide information relevant for mRNA processing, stability, and/or expression, as well as direct the termination of transcription and subsequent functions involved in proper mRNA processing, including posttranscriptional cleavage and polyadenylation.

The term “gene” refers to a coding region operably joined to appropriate regulatory sequences capable of regulating the expression of the polypeptide in some manner. A gene includes untranscribed and/or untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

A (polynucleotide) “construct” refers to a nucleic acid at least partly created by recombinant methods. The term “DNA construct” is referring to a polynucleotide construct consisting of deoxyribonucleotides. The construct may be single-stranded or preferably double-stranded. The construct may be circular or linear. The skilled worker is familiar with a variety of ways to obtain and generate a DNA construct.

Constructs can be prepared by means of customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular. Biology, Greene Publishing Assoc. and Wiley Interscience (1987).

When used in reference to a double-stranded nucleic acid sequence such as a cDNA or genomic clone, the term “substantially homologous” or “substantial homologue” refers to any probe which can hybridize to either or both strands of the double-stranded nucleic acid sequence under conditions of low stringency as described infra. When used in reference to a single stranded nucleic acid sequence, the term “substantially homologous” refers to any probe which can hybridize to the single-stranded nucleic acid sequence under conditions of low stringency as described infra.

The term “hybridization” as used herein includes any process by which a strand of nucleic acid joins with a complementary strand through base pairing. (Coombs 1994). Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein, the term “Tm” is used in reference to the “melting temperature”. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41 (% G+C), when a nucleic acid is in aqueous solution at 1 M NaCl [see e.g., Anderson and Young, Quantitative Filter Hybridization, in Nucleic Acid Hybridization (1985)]. Other references include more sophisticated computations which take structural as well as sequence characteristics into account for the calculation of Tm.

Low stringency conditions when used in reference to nucleic acid hybridization comprise conditions equivalent to binding or hybridization at 68° C. in a solution consisting of 5×SSPE (43.8 g/L NaCl, 6.9 g/LNaH2PO4. H20 and 1.85 g/L EDTA, pH adjusted to 7.4 with NaOH), 1% SDS, 5×Denhardt's reagent [50×Denhardt's contains the following per 500 mL: 5 gFicoll (Type 400, Pharmacia), 5 g BSA (Fraction V; Sigma)] and 100 ug/mL denatured salmon sperm DNA followed by washing in a solution comprising 0.2×SSPE, and 0.1% SDS at room temperature when a DNA probe of about 100 to about 1000 nucleotides in length is employed.

High stringency conditions when used in reference to nucleic acid hybridization comprise conditions equivalent to binding or hybridization at 68° C. in a solution consisting of 5×SSPE, 1% SDS, 5×Denhardt's reagent and 100 μg/mL denatured salmon sperm DNA followed by washing in a solution comprising 0.1×SSPE, and 0.1% SDS at 68 C when a probe of about 100 to about 1000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

The DSBI is encoded by a construct that may preferably be a nucleic acid construct. The DSBI enzyme is generated using an expression cassette that comprises the nucleic acid encoding a DSBI enzyme. The cassette is introduced into a plant cell or a plant. The term “expression cassette”—for example when referring to the expression cassette for the DSBI enzyme, but also with respect to any other sequence to be expressed in accordance with the present invention—means those constructs in which the “coding sequence” DNA to be expressed is linked operably to at least one genetic control element which enables or regulates its expression (i.e. transcription and/or translation). Here, expression may be for example stable or transient, constitutive or inducible. For introducing it, the skilled worker may resort to various direct methods (for example transfection, particle bombardment, microinjection) or indirect methods (for example infection with agrobacteria or viruses), all of which are detailed further below.

The following specifications about the expression cassettes, genetic control elements, promoters, enhancers etc. refer to the constructs encoding the DSBI as well as any other nucleic acid sequence which may possibly be expressed in the scope of this invention, such as the sequence which is to be excised, the marker gene sequence, the gene for resistance to antibiotics or herbicides etc.

A construct which is used for the transformation according to method step a) of the present invention may be any nucleic acid molecule which encodes a DNA double strand break inducing enzyme operably linked to at least one genetic control element. Preferably the construct encoding a DNA double strand break-inducing enzyme is selected from the group consisting of a vector, a plasmid, a cosmid, a bacterial construct or a viral construct.

A vector is a genetic construct that can be introduced into a cell. There are for example cloning vectors, expression vectors, gene fusion vectors, shuttle vectors, targeting vectors etc. If the construct is a vector, the vector is preferably selected from the group consisting of pCB series, pLM series, pJB series, pCER series, pEG series, pBR series, pUC series, M13mp series and pACYC series.

A plasmid is a circular DNA double strand molecule. It may be designed to allow the cloning and/or expression of DNA with recombinant DNA techniques. A cosmid (first described by Collins J. and Hohn B. in Proc. Natl. Acad. Sci. USA 1978 September; 75(9):4242-6) is a vector derived from the bacterial X virus (phage). It usually contains at least one or two cohesive (“cos”) sites. The cloning capacity of a cosmid is up to about 47 kb.

The term “about” is used herein to mean approximately, roughly, around, or in the region of. When the term “about” is used in conjunction with a numerical range, it modifies that range by extending the boundaries above and below the numerical values-set forth. In general, the term “about” is used herein to modify a numerical value above and below the stated value by a variance of 20 percent up or down (higher or lower), preferably 15 percent, more preferably 10 percent and most preferably 5 percent.

“Operable linkage” is generally understood as meaning an arrangement in which a genetic control sequence is capable of exerting its function with regard to a nucleic acid sequence to be expressed, for example while encoding a DSBI enzyme. Function, in this context, may mean for example control of the expression, i.e. transcription and/or translation, of the nucleic acid sequence, for example one encoding a DSBI enzyme. Control, in this context, encompasses for example initiating, increasing, governing or suppressing the expression, i.e. transcription and, if appropriate, translation. Controlling, in turn, may be, for example, tissue- and/or time-specific. It may also be inducible, for example by certain chemicals, stress, pathogens and the like.

Operable linkage is understood as meaning for example the sequential arrangement of a promoter, of the nucleic acid sequence to be expressed—in the present case e.g. one encoding a DSBI enzyme—and, if appropriate, further regulatory elements such as, for example, a terminator, in such a way that each of the regulatory elements can fulfill its function when the nucleic acid sequence—for example one encoding a DSBI enzyme—is expressed.

This does not necessarily require a direct linkage in the chemical sense. Genetic control sequences such as, for example, enhancer sequences are also capable of exerting their function on the target sequence from positions located at a distance or indeed other DNA molecules. Preferred arrangements are those in which the nucleic acid sequence to be expressed—for example one encoding a DSBI enzyme—is positioned after a sequence acting as promoter so that the two sequences are linked covalently to one another. The distance between the promoter sequence and the nucleic acid sequence—for example one encoding a DSBI enzyme—is preferably less than 200 base pairs, especially preferably less than 100 base pairs, very especially preferably less than 50 base pairs.

The skilled worker is familiar with a variety of ways in order to obtain such an expression cassette. For example, it is preferably prepared by directly fusing a nucleic acid sequence which acts as promotor with a nucleotide sequence to be expressed—for example one encoding a DSBI enzyme. Operable linkage can be achieved by means of customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

However, an expression cassette may also be constructed in such a way that the nucleic acid sequence to be expressed (for example one encoding a DSBI enzyme) is brought under the control of an endogenous genetic control element, for example a promotor, for example by means of homologous recombination or else by random insertion. Such constructs are likewise understood as being expression cassettes for the purposes of the invention.

The skilled worker furthermore knows that nucleic acid molecules may also be expressed using artificial transcription factors of the zinc finger protein type (Beerli R R et al. (2000) Proc Natl Acad Sci USA 97(4):1495-500). These factors can be adapted to suit any sequence region and enable expression independently of certain promotor sequences.

The term “genetic control sequences” is to be understood in the broad sense and refers to all those sequences that affect the coming into existence, or the function, of the expression cassette according to the invention. For example, genetic control sequences ensure transcription and, if appropriate, translation in the organism. Preferably, the expression cassettes according to the invention encompass 5′-upstream of the respective nucleic acid sequence to be expressed a promotor and 3′-downstream a terminator sequence as additional genetic control sequence, and, if appropriate, further customary regulatory elements, in each case in operable linkage with the nucleic acid sequence to be expressed. Genetic control sequences are described, for example, in “Goeddel; Gene Expression Technology: Methods in Enzymology 185, Academic Press, San Diego, Calif. (1990)” or “Gruber and Crosby, in: Methods in Plant Molecular Biology and Biotechnology, CRC Press, Boca Raton, Fla., eds.: Glick and Thompson, Chapter 7, 89-108” and the references cited therein.

Examples of such control sequences are sequences to which inductors or repressors bind and thus regulate the expression of the nucleic acid. The natural regulation of the sequences before the actual structural genes may still be present in addition to these novel control sequences or instead of these sequences and, if appropriate, may have been genetically modified in such a way that the natural regulation has been switched off and gene expression increased. However, the expression cassette may also be simpler in construction, that is to say no additional regulatory signals are inserted before the abovementioned genes, and the natural promotor together with its regulation is not removed. Instead, the natural control sequence is mutated in such a way that regulation no longer takes place and gene expression is increased. These modified promotors may also be placed on their own before the natural genes for increasing the activity.

A variety of control sequences are suitable, depending on the host organism or starting organism described in greater detail herein, which, owing to the introduction of the expression cassettes or vectors, becomes a genetically modified, or transgenic, organism.

“Transgene”, “transgenic” or “recombinant” refers to a polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples as described herein.

The term “transgenic” or “recombinant” when used in reference to a cell refers to a cell which contains a transgene, or whose genome has been altered by the introduction of a transgene.

The term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

A preferred promotor is, in principle, any promotor that is capable of controlling the expression of genes, in particular foreign genes, in plants. Preferred promotors are those that enable constitutive expression in plants (Benfey et al. (1989) EMBO J. 8:2195-2202). A promotor that is preferably used is, in particular, a plant promotor or a promotor derived from a plant virus. Especially preferred is the promotor of the cauliflower mosaic virus 35S transcript (Franck et al. (1980) Cell 21:285-294; Odell et al. (1985) Nature 313:810-812; Shewmaker et al. (1985) Virology 140:281-288; Gardner et al. 1986, Plant Mol. Biol. 6, 221-228) or the 19S CaMV promotor (U.S. Pat. No. 5,352,605 and WO 84/02913). It is known that this promotor comprises a variety of recognition sequences for transcriptional effectors that, in their totality, bring about permanent and constitutive expression of the gene introduced (Benfey et al. (1989) EMBO J. 8:2195-2202). A further suitable constitutive promotor is the Rubisco small subunit (SSU) promotor (U.S. Pat. No. 4,962,028). A further example of a suitable promotor is the leguminB promotor (GenBank Acc.-No.: X03677). Further preferred constitutive promotors are, for example, the Agrobacterium nopaline synthase promotor, the TR dual promotor, the Agrobacterium OCS (octopine synthase) promotor, the ubiquitin promotor (Holtorf S et al. (1995) Plant Mol Biol 29:637-649), the promoters of the vacuolar ATPase subunits, or the promotor of a wheat proline-rich protein (WO 91/13991).

The expression cassettes may also comprise an inducible, preferably a chemically inducible, promotor (Aoyama T and Chua N H (1997) Plant J 11:605-612; Caddick M X et al. (1998) Nat. Biotechnol 16:177-180; Review: Gatz, Annu Rev Plant Physiol Plant Mol Biol 1997, 48:89-108), by means of which the expression of the exogenous gene in the plant can be controlled at a specific point in time. Such promotors, such as, for example, the PRP1 promotor (Ward et al., Plant. Mol. Biol. 22 (1993), 361-366), a salicylic acid inducible promotor (WO 95/19443), a benzenesulfonamide inducible promotor (EP-A-0388186), a tetracycline inducible promotor (Gatz et al., (1992) Plant J. 2, 397-404), an abscisic acid inducible promotor (EP-A 335528), a salicylic acid inducible promotor (WO 95/19443) or an ethanol-(Salter M G et al. (1998) Plant J. 16:127-132) or cyclohexanone inducible (WO 93/21334) promotor may likewise be used.

In an especially preferred embodiment, nucleic acid encoding the DSBI enzyme, in particular, is expressed under the control of an inducible promotor. This leads to a controlled, governable expression and deletion—for example in plants—, and any potential deleterious effects caused by a constitutive expression of a DSBI enzyme are avoided.

Other preferred promotors are promoters induced by biotic or abiotic stress, such as, for example, the pathogen-inducible promotor of the PRP1 gene (Ward et al., Plant Mol Biol 1993, 22: 361-366), the tomato heat-inducible hsp80 promotor (U.S. Pat. No. 5,187,267), the potato chill-inducible alpha-amylase promotor (WO 96/12814) or the wound-induced pinII promotor (EP375091). Other preferred promoters are promoters with specificity for the anthers, ovaries, pollen, the meristem, flowers, leaves, stems, roots and seeds. A development-regulated promotor is, inter alia, described by Baerson et al. (Baerson S R, Lamppa G K (1993) Plant Mol Biol 22(2):255-67).

Especially preferred promoters are those that ensure expression in tissues or plant parts in which the biosynthesis of starch and/or oils or their precursors takes place or in which the products are advantageously accumulated. The cellular locations for starch biosynthesis are the chloroplasts of the leaves or the amyloplasts of the storage organs such as seeds, fruits or tubers. Within these organs, it is predominantly the cells of the endosperm or the cotyledons of the embryo in which synthesis takes place. Preferred promotors are thus in addition to the above-mentioned constitutive promotors in particular seed-specific promotors such as, for example, the phaseolin promotor (U.S. Pat. No. 5,504,200, Bustos M M et al., Plant Cell. 1989; 1(9):839-53), the promotor of the 2S albumin gene (Joseffson L G et al. (1987) J Biol Chem 262: 12196-12201), the legumin promotor (Shirsat A et al. (1989) Mol Gen Genet. 215(2):326-331), the USP (unknown seed protein) promotor (Bumlein H et al. (1991) Molecular & General Genetics 225(3):459-67), the napin gene promotor (U.S. Pat. No. 5,608,152; Stalberg K, et al. (1996) L. Planta 199: 515-519), the sucrose binding protein promotor (WO 00/26388) or the legumin B4 promotor (LeB4; Bumlein H et al. (1991) Mol Gen Genet. 225:121-128; Baeumlein et al. (1992) Plant Journal 2(2):233-239; Fiedler U et al. (1995) Biotechnology (NY) 13(10):1090-1093), the Ins Arabidopsis oleosin promotor (WO9845461), the Brassica Bce4 promotor (WO 91/13980). Further suitable seed-specific promoters are those of the genes encoding the “high-molecular-weight glutenin” (HMWG), gliadin, branching enzyme, ADP-glucose pyrophosphatase (AGPase) or starch synthase. Furthermore preferred promoters are those which enable seed-specific expression in monocots such as maize, barley, wheat, rye, rice and the like. Promotors that may advantageously be employed are the promotor of the lpt2 or lpt1 gene (WO 95/15389, WO 95/23230) or the promotors described in WO 99/16890 (promoters of the hordein gene, the glutelin gene, the oryzin gene, the prolamine gene, the gliadin gene, the glutelin gene, the zein gene, the kasirin gene or the secalin gene).

Promotors which are preferred as genetic control elements are, furthermore, pollen specific promoters such as, for example, the promotor of the B. campestris bgp1 gene (GenBank Acc.-No: X68210; Xu H et al. (1993) Mol Gen Genet. 239(1-2):58-65; WO 94/13809), of the Oryza sativa ory s1 gene (GenBank Acc.-No.: AJ012760; Xu H et al. (1995) Gene 164 (2):255-259), of the pollen-specific maize gene ZM13 (Hamilton D A et al. (1998) Plant Mol Biol 38(4):663-669; U.S. Pat. No. 5,086,169), of the B. napus gene Bp10 (GenBank Acc.-No.: X64257; Albani D (1992) Plant J 2(3):331-342; U.S. Pat. No. 6,013,859), and functional combinations of such promoters. Other preferred promoters are the Lcg1 promotor for cell-specific expression in the male gametes (WO 99/05281; XU H et al. (1999) Proc. Natl. Acad. Sci. USA Vol. 96:2554-2558) and the promotor of the AtDMC1 gene (Klimyuk V I et al. (1997) Plant J. 11(1):1-14). Further suitable promotors are, for example, specific promotors for tubers, storage roots or roots such as, for example, the class I patatin promotor (B33), the potato cathepsin D inhibitor promotor, the starch synthase (GBSS1) promotor or the sporamin promotor, and fruit-specific promoters such as, for example, the tomato fruit-specific promotor (EP-A 409625).

Promotors that are furthermore suitable are those which ensure leaf specific expression. Promotors which may be mentioned are the potato cytosolic FBPase promotor (WO 98/18940), the Rubisco (ribulose-1,5-bisphosphate carboxylase) SSU (small subunit) promoter, the potato ST-LSI promotor (Stockhaus et al. (1989) EMBO J. 8(9):2445-2451) or functional combinations of such promoters. Other preferred promotors are those that govern expression in seeds and plant embryos. Further suitable promoters are, for example, fruit-maturation-specific promotors such as, for example, the tomato fruit-maturation-specific promotor (WO 94/21794), flower-specific promotors such as, for example, the phytoene synthase promotor (WO 92/16635) or the promotor of the P-rr gene (WO 98/22593) or another node-specific promotor as described in EP-A 249676 may be used advantageously.

In principle, all natural promotors together with their regulatory sequences, such as those mentioned above, may be used for the method according to the invention. In addition, synthetic promotors may also be used advantageously. Genetic control sequences also encompass further promoters, promotor elements or minimal promotors capable of modifying the expression-specific characteristics. Thus, for example, the tissue-specific expression may take place in addition as a function of certain stress factors, owing to genetic control sequences. Such elements are, for example, described for water stress, abscisic acid (Lam E and Chua N H (1991) J Biol Chem 266(26):17131-17135) and heat stress (Schoffl F et al. (1989) Molecular & General Genetics 217(2-3):246-53). Furthermore, other promotors that enable expression in further plant tissues or other organisms, such as, for example, E. coli bacteria, may be linked operably with the nucleic acid sequence to be expressed. Plant promotors that are suitable are, in principle, all of the above-described promotors.

Preferably the promoter of the present invention is selected from the group consisting of constitutive promoters, development-dependent promoters, plant virus derived promoters, inducible promoters, chemically inducible promoters, biotic or abiotic stress inducible promoters, pathogen inducible promoters, tissue specific promoters, promoters with specificity for the embryo, scutellum, endosperm, embryo axis, anthers, ovaries, pollen, meristem, flowers, leaves, stems, roots, seeds, fruits and/or tubers, promoters which enable seed specific expression in monocotyledons including maize, barley, wheat, rye and rice, super promoters, and functional combinations of such promoters.

More preferably the promoter is selected from the group consisting of a ubiquitin promoter, sugarcane bacilliform virus promoter, phaseolin promoter, 35S CaMV promoter, 19S CaMV promoter, short or long USB promoter, Rubisco small subunit promoter, legumin B promoter, nopaline synthase promoter, TR dual promoter, octopine synthase promoter, vacuolar ATPase subunit promoter, proline-rich protein promoter, PRP1 promoter, benzenesulfonamide-inducible promoter, tetracycline-inducible promoter, abscisic acid-inducible promoter, salicylic acid-inducible promoter, ethanol inducible promoter, cyclohexanone inducible promoter, heat-inducible hsp80 promoter, chill-inducible alpha-amylase promoter, wound-induced pinII promoter, 2S albumin promoter, legumin promoter, unknown seed protein promoter, napin promoter, sucrose binding protein promoter, legumin B4 promoter, oleosin promoter, Bce4 promoter, high-molecular-weight glutenin promoter, gliadin promoter, branching enzyme promoter, ADP-glucose pyrophosphatase promoter, synthase promoter, bgp1 promoter, lpt2 or lpt1 promoter, hordein promoter, glutelin promoter, oryzin promoter, prolamine promoter, gliadin promoter, glutelin promoter, zein promoter, kasirin promoter, secalin promoter, ory s1 promoter, ZM13 promoter, Bp10 promoter, Lcg1 promoter, AtDMC1 promoter, class I patatin promoter, B33 promoter, cathepsin D inhibitor promoter, starch synthase promoter, GBSS1 promoter, sporamin promoter, tomato fruit-specific promoter, cytosolic FBPase promoter, ST-LSI promoter, CP12 promoter, CcoMT1 promoter, HRGP promoter, super promoter, promoters in combination with an intron-mediated enhancement (IME) conferring intron (preferably located between the promoter and the “structural” gene, i.e. the sequence to be expressed), and functional combinations of such promoters.

Most preferably the promoter comprises a nucleic acid sequence as depicted in nucleotides 1 to 1112 of SEQ ID NO: 6.

Genetic control sequences furthermore also encompass the 5′-untranslated region, introns or the noncoding 3′-region of genes. It has been demonstrated that they may play a significant role in the regulation of gene expression. Thus, it has been demonstrated that 5′-untranslated sequences are capable of enhancing the transient expression of heterologous genes. Furthermore, they may promote tissue specificity (Rouster J et al., Plant J. 1998, 15: 435-440.). Conversely, the 5′-untranslated region of the opaque-2 gene suppresses expression. Deletion of the region in question leads to an increased gene activity (Lohmer S et al., Plant Cell 1993, 5:65-73).

Genetic control sequences may also encompass ribosome-binding sequences for initiating translation. This is preferred in particular when the nucleic acid sequence to be expressed does not provide suitable sequences or when they are not compatible with the expression system. Genetic control sequences are furthermore understood as also encompassing sequences that create fusion proteins comprising a signal peptide sequence directing subcellular localization of a protein.

The expression cassette can advantageously comprise one or more of what are known as enhancer sequences in operable linkage with the promotor, which enable the increased transgenic expression of the nucleic acid sequence. Additional advantageous sequences, such as further regulatory elements or terminators, may also be inserted at the 3′-end of the nucleic acid sequences to be expressed recombinantly. One or more copies of the nucleic acid sequences to be expressed recombinantly may be present in the gene construct.

Polyadenylation signals which are suitable as genetic control sequences are plant polyadenylation signals, preferably those which correspond essentially to T-DNA polyadenylation signals from Agrobacterium tumefaciens, in particular of gene 3 of the T-DNA (octopine synthase) of the Ti plasmids pTiACHS (Gielen et al., EMBO J. 3 (1984), 835 et sec.) or functional equivalents thereof. Examples of particularly suitable terminator sequences are the OCS (octopine synthase) terminator and the NOS (nopaline synthase) terminator.

The term “transformation” or “transforming” as used herein refers to the introduction of a nucleic acid molecule (e.g. a transgene) into a plant cell. Preferably the transformation method is selected from the group consisting of Agrobacterium mediated transformation, biolistic transformation (gene gun), protoplast transformation, polyethylene glycol transformation, electroporation, sonication, microinjection, macro injection, vacuum filtration, infection, incubation of dried embryos in DNA-containing solution, osmotic shock, silica/carbon fibers, laser mediated transformation, meristem transformation (floral dip, vacuum infiltration), and pollen transformation.

Methods for transforming plant cells/plants and for regenerating plants from plant tissues or plant cells with which the skilled worker is familiar are exploited for transient or stable transformation. Suitable direct methods of DNA delivery are especially those for either protoplast transformation or for the intact cells and tissues by means of polyethylene-glycol-induced DNA uptake, biolistic methods such as the gene gun (“particle bombardment” method), electroporation, the incubation of dry embryos in DNA-containing solution, sonication and microinjection, the micro- or macroinjection into tissues or embryos, tissue electroporation, incubation of dry embryos in DNA-containing solution or vacuum infiltration of seeds. In the case of injection or electroporation of DNA into plant cells, the plasmid used need not meet any particular requirement. Simple plasmids such as those of the pUC series may be used with or without linearization. If intact plants are to be regenerated from the transformed cells, the presence of an additional selectable marker gene on the plasmid is useful.

Any plant tissue may act as target material. Likewise, expression may take place in callus, embryogenic tissue or somatic embryos.

In addition to these “direct” transformation techniques, transformation can also be carried out by means of Agrobacterium tumefaciens or Agrobacterium rhizogenes. These strains contain a plasmid (Ti or Ri plasmid). Part of this plasmid, termed T-DNA (transferred DNA), is transferred to the plant following agrobacterial infection and integrated into the genome of the plant cell.

The term “Agrobacterium” refers to a soil-borne, Gram-negative, rod-shaped phytopathogenic bacterium that causes crown gall. The term “Agrobacterium” includes, but is not limited to, the strains Agrobacterium tumefaciens, (which typically causes crown gall in infected plants), and Agrobacterium rhizogenes (which causes hairy root disease in infected host plants).

Infection of a plant cell with Agrobacterium generally results in the production of opines (e.g., opaline, agropine, octopine etc.) by the infected cell.

The terms “infecting” and “infection” with a bacterium refer to co-incubation of a target biological sample, (e.g., cell, tissue, etc.) with the bacterium under conditions such that nucleic acid sequences contained within the bacterium are introduced into one or more cells of the target biological sample.

In general, transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme linked immunosorbent assay (ELISA), which detects the presence of a polypeptide encoded by one or more of the transgenes. Alternatively, transient transformation may be detected by assessing the activity of the protein encoded by the transgene as demonstrated herein (e.g., histochemical assay of GUS enzyme activity by staining with X-glucoronidase which gives a blue precipitate in the presence of the GUS enzyme; or a chemiluminescent assay of GUS enzyme activity using the GUS-Light kit (Tropix)). The term “transient transformant” refers to a cell that has transiently contained one or more transgenes in the cell without incorporating the introduced DNA into its genome.

In contrast, the term “stable transformation” or “stably transformed” refers to the introduction and integration of one or more transgenes into the genome of a cell, preferably resulting in chromosomal integration and stable heritability through mitosis and meiosis. Stable transformation of a cell may be detected by Southern blot hybridization of genomic DNA of the cell with nucleic acid sequences that are capable of binding to one or more of the transgenes after a period of time when transgene integration into the plant genome occurs. Alternatively, stable transformation of a cell may also be detected by the polymerase chain reaction of genomic DNA of the cell to amplify transgene sequences. The term “stable transformant” refers to a cell that has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression that may exhibit variable properties with respect to meiotic stability.

The DNA constructs can be introduced into cells, either in culture or in the organs of a plant by a variety of conventional techniques. For example, the DNA constructs can be introduced directly to plant cells using ballistic methods such as DNA particle bombardment, or the DNA construct can be introduced using techniques such as electroporation and microinjection of a cell. Particle mediated transformation techniques (also known as “biolistics”) are described in, e.g., Klein et al. (1987) Nature 327: 70-73; Vasil V et al. (1993) Bio/Technol 11: 1553-1558; and Becker D et al. (1994) Plant J 5: 299-307. These methods involve penetration of cells by small particles with the nucleic acid either within the matrix of small beads or particles, or on the surface.

The terms “bombarding”, “bombardment”, and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807), and are commercially available (e.g., the helium gas-driven microprojectile accelerator (PDS-1000/He) (BioRad).

The biolistic PDS-1000 Gene Gun (Biorad, Hercules, Calif.) uses helium pressure to accelerate DNA-coated gold or tungsten rnicrocarriers toward target cells. The process is applicable to a wide range of tissues and cells from organisms, including plants. The term “microwounding” when made in reference to plant tissue refers to the introduction of microscopic wounds in that tissue. Microwounding may be achieved by, for example, particle bombardment as described herein.

Microinjection techniques are known in the art and are well described in the scientific and patent literature. Also, the cell can be permeabilized chemically, for example using polyethylene glycol, so that the DNA can enter the cell by diffusion. The DNA can also be introduced by protoplast fusion with other DNA-containing units such as minicells, cells, lysosomes or liposomes. The introduction of DNA constructs using polyethylene glycol (PEG) precipitation is described in Paszkowski et al. (1984) EMBO J. 3: 2717.

Liposome-based gene delivery is e.g., described in WO 93/24640; Mannino and Gould-Fogerite (1988) BioTechniques 6 (7): 682-691; U.S. Pat. No. 5,279,833; WO 91/06309; and Felgner et al. (1987) Proc Natl Acad Sci USA 84:7413-7414).

Another suitable method of introducing DNA is electroporation, where an electrical pulse is used to reversibly permeabilize the cells. Electroporation techniques are described in Fromm et al. (1985) Proc Natl Acad Sci USA 82: 5824. PEG-mediated transformation and electroporation of plant protoplasts are also discussed in Lazzeri P (1995) Methods Mol. Biol. 49: 95-106. Preferred general methods that may be mentioned are the calcium-phosphate-mediated transfection, the DEAE-dextran-mediated transfection, the cationic lipid mediated transfection, electroporation, transduction and infection. Such methods are known to the skilled worker and described, for example, in Davis et al., Basic Methods In Molecular Biology (1986). For a review of gene transfer methods for plant and cell cultures, see, Fisk et al. (1993) Scientia Horticulturae 55: 5-36 and Potrykus (1990) CIBA Found Symp 154: 198. Methods are known for introduction and expression of heterologous genes in both monocot and dicot plants. See, e.g., U.S. Pat. No. 5,633,446, U.S. Pat. No. 5,317,096, U.S. Pat. No. 5,689,052, U.S. Pat. No. 5,159,135, and U.S. Pat. No. 5,679,558; Weising et al. (1988) Ann. Rev. Genet. 22: 421-477.

Transformation of monocots in particular can use various techniques including electroporation (e.g., Shimamoto et al. (1992) Nature 338: 274-276); biolistics (e.g., EP-A1270, 356); and Agrobacterium (e.g., Bytebier et al. (1987) Proc Natl Acad Sci USA. 84: 5345-5349). In particular, Agrobacterium mediated transformation is now a highly efficient transformation method in monocots (Hiei et al. (1994) Plant J 6: 271-282). A generation of fertile transgenic plants can be achieved using this approach in the cereals maize, rice, wheat, oat, and barley (reviewed in Shimamoto K (1994) Current Opinion in Biotechnology 5: 158-162; Vasil et al. (1992) Bio/Technology 10: 667-674; Vain et al. (1995) Biotechnology Advances 13(4): 653-671; Vasil (1996) Nature Biotechnology 14: 702; Wan & Lemaux (1994) Plant Physio. 104: 37-48) Other methods, such as microprojectile or particle bombardment (U.S. Pat. No. 5,100,792, EP-A-444 882, EP-A-434 616), electroporation (EP-A 290 395, WO 87/06614), microinjection (WO 92/09696, WO 94/00583, EP-A 331 083, EP-A 175 966, Green et al. (1987) Plant Tissue and Cell Culture, Academic Press) direct DNA uptake (DE 4005152, WO 90/12096, U.S. Pat. No. 4,684,611), liposome mediated DNA uptake (e.g. Freeman et al. (1984) Plant Cell Physiol 2 9: 1353), or the vortexing method (e.g., Kindle (1990) Proc Natl Acad Sci USA 87: 1228) may be preferred where Agrobacterium transformation is inefficient or ineffective.

In particular, transformation of gymnosperms, such as conifers, may be performed using particle bombardment 20 techniques (Clapham D et al. (2000) Scan J For Res 15: 151-160). Physical methods for the transformation of plant cells are reviewed in Oard, (1991) Biotech. Adv. 9: 1-11. Alternatively, a combination of different techniques may be employed to enhance the efficiency of the transformation process, e.g. bombardment with Agrobacterium coated microparticles (EP-A-486234) or microprojectile bombardment to induce wounding followed by co-cultivation with Agrobacterium (EP-A-486233).

The expression cassette for the DSBI enzyme is preferably integrated into specific plasmids, either into a shuttle, or intermediate, a vector or into a binary vector. If, for example, a Ti or Ri plasmid is to be used for the transformation, at least the right border, but in most cases the right and the left border, of the Ti or Ri plasmid T-DNA is linked with the expression cassette to be introduced as a flanking region. Binary vectors are preferably used. Binary vectors are capable of replication both in E. coli and in Agrobacterium. They contain a selection marker gene and a linker or polylinker flanked by the right or left T-DNA flanking sequence. They can be transformed directly into Agrobacterium (Holsters et al., Mol. Gen. Genet. 163 (1978), 181-187). The selection marker gene permits the selection of transformed agrobacteria and is, for example, the nptII gene, which imparts resistance to kanamycin. The agrobacterium, which acts as host organism in this case, should already contain a plasmid with the vir region. The latter is required for transferring the T-DNA to the plant cell. An agrobacterium thus transformed can be used for transforming plant cells.

The use of Agrobacterium tumefaciens for the transformation of plants using tissue culture explants has been described by Horsch et al. (Horsch R B (1986) Proc Natl Acad Sci USA 83(8):2571-2575), Fraley et al. (Fraley et al. 1983, Proc. Natl. Acad. Sci. USA 80, 4803-4807) and Bevans et al. (Bevans et al. 1983, Nature 304, 184-187).

Many strains of Agrobacterium tumefaciens are capable of transferring genetic material, such as, for example, the strains [pEHA101], EHA105[pEHA105], LBA4404[pAL4404], C58C1[pMP90] and C58C1[pGV2260]. The strain EHA101[pEHA101] has been described by Hood et al. (Hood E E et al. (1996) J Bacteriol 168(3):1291-1301), the strain EHA105[pEHA105] by Hood et al. (Hood et al. 1993, Transgenic Research 2, 208-218), the strain LBA4404[pAL4404] by Hoekema et al. (Hoekema et al. 1983, Nature 303, 179-181), the strain C58C1[pMP90] by Koncz and Schell (Koncz and Schell 1986, Mol. Gen. Genet. 204, 383-396), and the strain C58C1[pGV2260] by Deblaere et al. (Deblaere et al. 1985, Nucl. Acids Res. 13, 4777-4788).

For Agrobacterium-mediated transformation of plants, the DNA construct of the invention may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the bacteria infect the cell. Agrobacterium tumefaciens mediated transformation techniques are well described in the scientific literature. See, for example, Horsch et al. (1984) Science 233: 496-498, Fraley et al. (1983) Proc Natl Acad Sci USA 80:4803-4807, Hooykaas (1989) Plant Mol Biol 13: 327-336, Horsch R B (1986) Proc Natl Acad Sci USA 83 (8):2571-2575), Bevans et al. (1983) Nature 304:184-187, Bechtold et al. (1993) Comptes Rendus De L′Academie Des Sciences Serie III-Sciences De La Vie-Life Sciences 316: 1194-1199, Valvekens et al. (1988) Proc Natl Acad Sci USA 85: 5536-5540.

The agrobacterial strain employed for the transformation comprises, in addition to its disarmed Ti plasmid, a binary plasmid with the T-DNA to be transferred, which usually comprises a gene for the selection of the transformed cells and the gene to be transferred. Both genes must be equipped with transcriptional and translational initiation and termination signals. The binary plasmid can be transferred into the agrobacterial strain for example by electroporation or other transformation methods (Mozo & Hooykaas 1991, Plant Mol. Biol. 16, 917-918). Coculture of the plant explants with the agrobacterial strain is usually performed for two to three days.

A variety of vectors could, or can, be used. In principle, one differentiates between those vectors which can be employed for the agrobacterium-mediated transformation or agroinfection, i.e. which comprise the expression cassette, for the expression of the DSBI enzyme within a T-DNA, which indeed permits stable integration of the T-DNA into the plant genome. Moreover, border-sequence-free vectors may be employed, which can be transformed into the plant cells for example by particle bombardment, where they can lead both to transient and to stable expression.

The use of T-DNA for the transformation of plant cells has been studied and described intensively (EP 120516; Hoekema, In: The Binary Plant Vector System, Offsetdrukkerij Kanters B. V., Alblasserdam, Chapter V; Fraley et al., Crit. Rev. Plant. Sci., 4:1-46 and An et al., EMBO J. 4 (1985), 277-287). Various binary vectors are known, some of which are commercially available such as, for example, pBIN19 (Clontech Laboratories, Inc. USA).

To transfer the DNA to the plant cell, plant explants are cocultured with Agrobacterium tumefaciens or Agrobacterium rhizogenes. Starting from infected plant material (for example leaf, root or stalk sections, but also protoplasts or suspensions of plant cells), intact plants can be regenerated using a suitable medium that may contain, for example, antibiotics or biocides for selecting transformed cells. The plants obtained can then be screened for the presence of the DNA introduced, in this case the expression cassette for the DSBI enzyme according to the invention. As soon as the DNA has integrated into the host genome, the genotype in question is, as a rule, stable and the insertion in question is also found in the subsequent generations. As a rule, the expression cassette integrated contains a selection marker that confers a resistance to a biocide (for example a herbicide) or an antibiotic such as kanamycin, G 418, bleomycin, hygromycin or phosphinotricin and the like to the transformed plant. The selection marker permits the selection of transformed cells (McCormick et al., Plant Cell Reports 5 (1986), 81-84). The plants obtained can be cultured and hybridized in the customary fashion. Two or more generations should be grown in order to ensure that the genomic integration is stable and hereditary.

The abovementioned methods are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu Rev. Plant Physiol. Plant Molec. Biol. 42 (1991), 205-225). The construct to be expressed is preferably cloned into a vector that is suitable for the transformation of Agrobacterium tumefaciens, for example pBin19 (Bevan et al., Nucl. Acids Res. 12 (1984), 8711).

Agrobacterium-mediated transformation is suited best to dicotyledonous plant cells, and has been successfully optimized for certain monocotyledonous plant cells, whereas the direct transformation techniques are suitable for any cell type.

Transformed cells, i.e. those that comprise the DNA integrated into the DNA of the host cell, can be selected from untransformed cells if a selectable marker is part of the DNA introduced. A marker can be, for example, any gene that is capable of conferring a resistance to antibiotics or herbicides. Transformed cells that express such a marker gene are capable of surviving in the presence of concentrations of a suitable antibiotic or herbicide that kill an untransformed wild type. Various positive and negative selection markers are described hereinabove. Examples are the ahas (acetohydroxy acid synthase) gene, which confers resistance to sulfonylurea and imidazolinone herbicides, the bar gene, which confers resistance to the herbicide phosphinothricin (Rathore K S et al., Plant Mol. Biol. March 1993; 21(5):871-884), the nptII gene, which confers resistance to kanamycin, the hpt gene, which confers resistance to hygromycin, or the EPSP gene, which confers resistance to the herbicide Glyphosate.

As soon as a transformed plant cell has been generated, an intact plant can be obtained using methods known to the skilled worker. For example, callus cultures are used as starting material. The formation of shoot and root can be induced in this as yet undifferentiated cell biomass in the known fashion. The shoots obtained can be induced for root development under the suitable conditions. The recovered plants can then be cultured in vitro, and planted in soil.

After the transformation of a plant cell with a construct encoding a DSBI enzyme, a transgenic plant line is generated from the transformed plant cell. This generation occurs according to the methods known by the person skilled in the art. Usually, step b) of the present invention may be performed as follows.

For generating the intact transgenic plant containing the gene encoding a DSBI enzyme, the putative transgenic calli that are resistant to the chemical selection agent either D-serine or Imazethapyr (Pursuit™) depending on the selection marker gene used, are cultured on the regeneration medium containing the shoot promoting phytohormone (e.g. cytokinine) and also the selection agent. Shoot formed are transferred to the rooting medium in the presence of selection agent, but in the absence of phytohormones. The integration of the transgene in the generated putative transgenic plant genome is confirmed by routine molecular techniques such as Southern blot analysis, or PCR analysis.

In the following will be described the characteristics of the nucleic acid sequence to be excised by the action of the DSBI enzyme.

The term “nucleic acid sequence to be excised” refers to any nucleotide sequence of any length, the excision or deletion of which may be deemed desirable for any reason (e.g., confer improved qualities), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product, (e.g., promoter sequence, polyadenylation sequence, termination sequence, enhancer sequence, etc.).

Preferably, the length of the sequence to be excised is at least about 10 or at least about 50 base pairs, more preferably at least about 100 or at least about 500 base pairs, especially preferably at least about 1000 or at least about 5000 base or pairs, and most preferably at least about 10000 or at least about 50000 base pairs. Also, preferably, the length of the sequence to be excised is at most about 50000 or at most about 10000 base pairs, more preferably at most about 5000 or at most about 1000 base pairs, especially preferably at least about 500 or at least about 100 base pairs. Those lower and upper limits may be combined in any adequate way.

The nucleic acid to be excised may be initially part of a construct, a so-called “recombination construct”, which serves e.g. for the transformation of a plant cell or a plant in order to result in a plant line containing the nucleic acid sequence to be excised (see step d) of the method according to the invention).

The nucleic acid sequence(s) to be excised (for example the T-DNA region or parts thereof, or selection markers such as genes for resistance to antibiotics or herbicides) are deleted or excised from the genome of a plant in a predictable manner. The sequence to be eliminated comprises at least one recognition sequence for the site directed induction of a DNA double strand break (for example recognition sequences of rare-cleaving restriction enzymes) and is bordered at both sides by a repeated sequence (or “homologous” sequence). A double strand break is induced by an enzyme suitable for inducing DNA double strand breaks at the recognition sequence (a DSBI enzyme), which, in consequence, triggers the homologous recombination of the homologous sequences, and thus the deletion of any nucleic acid sequence located between the homologous sequences. The recognition sequence for the site directed induction of DNA double strand breaks is likewise deleted.

The term “recognition sequence” refers to a DNA sequence that is recognized by a DSBI as described above. A recognition sequence for the site directed induction of DNA double strand breaks generally refers to those sequences that, under the conditions in the plant cell or plant used in each case, enable the recognition and cleavage by the DSBI enzyme. The recognition sequence will typically be at least 10 base pairs long, is more usually 10 to 30 base pairs long, and in most embodiments, is less than 50 base pairs long. Recognition sequences for sequence specific DSBIs (e.g., homing endonucleases) are described in the art. Recognition sequences and organisms of origin of the respective DSBI enzymes can be taken, e.g., from WO 03/004695.

Also encompassed are minor deviations (degenerations) of the recognition sequence that still enable recognition and cleavage by the DSBI enzyme in question. Such deviations also in connection with different framework conditions such as, for example, calcium or magnesium concentration have been described (Argast G M et al. (1998) J Mol Biol 280: 345-353). Also encompassed are core sequences of these recognition sequences. It is known that the inner portions of the recognition sequences suffice for an induced double-strand break and that the outer ones are not absolutely relevant, but can codetermine the cleavage efficacy. Thus, for example, an 18 by core sequence can be defined for I-SceI:

| | |
|---|---|
| Recognition sequence of I-SceI: | |
| 5′-AGTTACGCTAGGGATAA\hat{ }CAGGGTAATATAG | (SEQ ID NO: 28) |
| 3′-TCAATGCGATCCC\hat{ }TATTGTCCCATTATATC | |
| Core sequence of I-SceI: | |
| 5′-TAGGGATAA\hat{ }CAGGGTAAT | (SEQ ID NO: 29) |
| 3′-ATCCC\hat{ }TATTGTCCCATTA | |

The sequences that are deleted or excised are those located between the two homology sequences (e.g. homology or repeated sequences called “A” and “B”). The skilled worker knows that he is not bound to specific sequences when performing recombination, but that any sequence can undergo homologous recombination with another sequence provided that sufficient length and homology exist.

“Homologous recombination” is a DNA recombination event occurring at and encouraged by the presence of two homologous (“repeated”) DNA sites, it leads to a rearrangement or reunion of the DNA sequences by crossing over in the region of identical sequence.

Referring to the “homology” or “repeated” sequences A and B, “sufficient length” preferably refers to sequences with a length of at least 20 base pairs, preferably at least 50 base pairs, especially preferably at least 100 base pairs, very especially preferably at least 250 base pairs, most preferably at least 500 base pairs.

Referring to the homology sequences A and B, “sufficient homology” preferably refers to sequences with at least 70%, preferably 80%, by preference at least 90%, especially preferably at least 95%, very especially preferably at least 99%, most preferably 100%, homology within these homology sequences over a length of at least 20 base pairs, preferably at least 50 base pairs, especially preferably at least 100 base pairs, very especially preferably at least 250 base pairs, most preferably at least 500 base pairs.

“Homology” between two nucleic acid sequences is understood as meaning the identity of the nucleic acid sequence over in each case the entire sequence length which is calculated by alignment with the aid of the program algorithm GAP (Wisconsin Package Version 10.0, University of Wisconsin, Genetics Computer Group (GCG), Madison, USA), setting the following parameters: 1 Gap Weight: 12 Length Weight: 4 Average Match: 2,912 Average Mismatch: −2,003.

In one embodiment, only one recognition sequence for the site-directed induction of DNA double strand breaks is located between the homology sequences A and B, so that the nucleic acid sequence to be excised (or the recombination construct employed for the transformation of a target plant or plant cell for the generation of a plant line of step d) of the method according to the invention) is constructed in the 5′- to 3′-orientation as follows:
a1) a first homology sequence A,
b1) a recognition sequence for the site-directed induction of DNA double strand breaks, and
a2) a second homology sequence B, the homology sequences A and B having a sufficient length and sufficient homology in order to enable efficient homologous recombination.

In another embodiment, a further nucleic acid sequence is located between the homology sequences A and B, so that the nucleic acid sequence to be excised (or the recombination construct employed for the transformation of a target plant or plant cell for the generation of a plant line of step d) of the method according to the invention) is constructed as follows in the 5′/3′-direction of:
a1) a first homology sequence A,
b1) a recognition sequence for the site-directed induction of DNA double strand breaks,
c) a further nucleic acid sequence, and
a2) a second homology sequence B, the homology sequences A and B having a sufficient length and sufficient homology in order to enable efficient homologous recombination.

The recognition sequence for the site-directed induction of DNA double strand breaks may also be located after or within the further nucleic acid sequence.

In a further embodiment, a second recognition sequence for the site-directed induction of double strand breaks is present after the further nucleic acid sequence. This embodiment is advantageous in particular in the case of homology sequences A and B which are further apart, or in the case of longer further nucleic acid sequences, since recombination efficacy is increased. In this embodiment, the nucleic acid sequence to be excised (or the recombination construct employed for the transformation of a target plant or plant cell for the generation of a plant line of step d) of the method according to the invention) is constructed as follows in a 5′- to 3′-orientation of:
a1) a first homology sequence A,
b1) a first recognition sequence for the site-directed induction of DNA double strand breaks, and
c) a further nucleic acid sequence, and
b2) a second recognition sequence for the site-directed induction of DNA double strand breaks, and
a2) a second homology sequence B, the homology sequences A and B having a sufficient length and sufficient homology in order to enable efficient homologous recombination.

Furthermore, other recognition sequences may also be present between the homology sequences A and B, in addition to the second recognition sequences for the site-directed induction of DNA double strand breaks. The individual recognition sequences (for example b1 or b2) for the site-directed induction of DNA double strand breaks may be identical or different, i.e. they may act as recognition sequence for an individual enzyme for the site-directed induction of DNA double strand breaks or else for a variety of enzymes. The embodiment in which the recognition sequences for the site-directed induction of DNA double strand breaks act as recognition sequence for an individual enzyme for the site-directed induction of DNA double strand breaks is preferred in this context.

The skilled worker is familiar with a variety of ways to obtain a recombination construct comprising the nucleic acid sequence to be excised and to obtain a plant line containing the nucleic acid sequence to be excised. The construct can be prepared by means of customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987). Preferably, the recombination construct according to the invention is generated by joining the above-mentioned essential constituents of the recombination construct together in the above-mentioned sequence using the recombination and cloning techniques with which the skilled worker is familiar, and the result is then introduced into the genome of a host plant.

Furthermore, the skilled worker is familiar with various ways in which the recombination construct according to the invention may be introduced into the genome of a plant cell or plant. In this context, the insertion may be directed (i e taking place at a defined insertion site) or undirected (i.e. taking place randomly). Suitable techniques are known to the skilled worker.

In addition to the elements described above with respect to the expression cassette of the DSBI enzyme (genetic control elements, promoter, enhancer etc.) the recombination construct (comprising the nucleic acid sequence to be excised) may encompass further nucleic acid sequences. Such nucleic acid sequences may preferably constitute expression cassettes. The following may be mentioned by way of example of the DNA sequences to be expressed in the expression constructs, but not by way of limitation:

### i) Positive Selection Markers:

Positive selection markers are genes whose presence conveys to a cell or plant the ability to persist or be identified in the presence of an otherwise harmful treatment. As pertaining to plant transformation, selection markers are required for selecting cells that have integrated and expressed any DNA of interest, e.g. the T-DNA. The selectable marker which has been introduced together with the expression construct can confer resistance to a biocide (for example a herbicide such as phosphinothricin, glyphosate or bromoxynil), a metabolism inhibitor such as 2-deoxyglucose-6-phosphate (WO 98/45456) or an antibiotic such as, for example, tetracyclines, ampicillin, kanamycin, G 418, neomycin, bleomycin or hygromycin to the cells which have successfully undergone recombination or transformation. The selection marker permits the selection of the transformed cells from untransformed cells (McCormick et al., Plant Cell Reports 5 (1986), 81-84). Especially preferred selection markers are those that confer resistance to herbicides. Examples of selection markers that may be mentioned are:
DNA sequences which encode phosphinothricin acetyltransferases (PAT), which acetylates the free amino group of the glutamine synthase inhibitor phosphinothricin (PPT) and thus brings about detoxification of the PPT (de Block et al. 1987, EMBO J. 6, 2513-2518) (also referred to as Bialophos® resistance gene (bar)),
5-enolpyruvylshikimate-3-phosphate synthase genes (EPSP synthase genes), which confer resistance to Glyphosate® (N-(phosphonomethyl)glycine),
the gox gene, which encodes the Glyphosate®-degrading enzyme (Glyphosate oxidoreductase),
the deh gene (encoding a dehalogenase which inactivates Dalapon®),
the mutated acetolactate synthases which are insensitive to sulfonylurea and imidazolinone,
bxn genes which encode Bromoxynil®-degrading nitrilase enzymes,
the kanamycin, or G418, resistance gene (NPTII). The NPTII gene encodes a neomycin phosphotransferase which reduces the inhibitory effect of kanamycin, neomycin, G418 and paromomycin owing to a phosphorylation reaction,
the DOG.sup.R1 gene. The DOG.sup.R1 gene has been isolated from the yeast Saccharomyces cerevisiae (EP 0 807 836). It encodes a 2-deoxyglucose-6-phosphate phosphatase which confers resistance to 2-DOG (Randez-Gil et al. 1995, Yeast 11, 1233-1240).
   ii) Negative, or counter, selection markers enable the identification and/or survival of cells lacking a specified gene function, for example the selection of organisms with successfully deleted sequences which encompass the marker gene (Koprek T et al. (1999) The Plant Journal 19(6):719-726). TK thymidine kinase (TK) and diphtheria toxin A fragment (DT-A), codA gene encoding a cytosine deaminase (Gleve A P et al. (1999) Plant Mol. Biol. 40(2):223-35; Pereat R I et al. (1993) Plant Mol. Biol. 23(4): 793-799; Stougaard J; (1993) Plant J 3:755-761), the cytochrome P450 gene (Koprek et al. (1999) Plant J. 16:719-726), genes encoding a haloalkane dehalogenase (Naested H (1999) Plant J. 18:571-576), the iaah gene (Sundaresan V et al. (1995) Genes & Development 9:1797-1810) or the tms2 gene (Fedoroff N V & Smith D L 1993, Plant J 3: 273-289).
   iii) Reporter genes which encode readily quantifiable proteins and which may also, via intrinsic color or enzyme activity, ensure the assessment of the transformation efficacy or of the location or timing of expression. Very especially preferred here are genes encoding reporter proteins (see also Schenborn E, Groskreutz D. Mol. Biotechnol. 1999; 13(1):29-44) such as:
“green fluorescence protein” (GFP) (Chui W L et al., Curr Biol 1996, 6:325-330; Leffel S M et al., Biotechniques. 23(5):912-8, 1997; Sheen et al. (1995) Plant Journal 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228).
chloramphenicoltransferase,
luciferase (Millar et al., Plant Mol Biol Rep 1992 10:324-414; Ow et al. (1986) Science, 234:856-859); permits the detection of bioluminescence,
beta-galactosidase, encodes an enzyme for which a variety of chromogenic substrates are available,
beta-glucuronidase (GUS) (Jefferson et al., EMBO J. 1987, 6, 3901-3907) or the uidA gene, which encodes an enzyme for a variety of chromogenic substrates,
R locus gene product: protein which regulates the production of anthocyanin pigments (red coloration) in plant tissue and thus makes possible the direct analysis of the promotor activity without the addition of additional adjuvants or chromogenic substrates (Dellaporta et al., In: Chromosome Structure and Function: Impact of New Concepts, 18th Stadler Genetics Symposium, 11:263-282, 1988),
beta-lactamase (Sutcliffe (1978) Proc Natl Acad Sci USA 75:3737-3741), enzyme for a variety of chromogenic substrates (for example PADAC, a chromogenic cephalosporin),
xylE gene product (Zukowsky et al. (1983) Proc Natl Acad Sci USA 80:1101-1105), catechol dioxygenase capable of converting chromogenic catechols,
alpha-amylase (Ikuta et al. (1990) Bio/technol. 8:241-242),
tyrosinase (Katz et al. (1983) J Gene Microbiol 129:2703-2714), enzyme which oxidizes tyrosine to give DOPA and dopaquinone which subsequently form melanine, which is readily detectable,
aequorin (Prasher et al. (1985) Biochem Biophys Res Commun 126(3):1259-1268), can be used in the calcium-sensitive bioluminescence detection.

The above-mentioned nucleic acids encoding markers and reporter genes can be comprised within the nucleic acid to be excised according to the invention. The same applies, e.g., for the T-DNA region or part thereof.

The recombination construct and any vectors derived from it may comprise further functional elements. The term “further functional elements” is to be understood in the broad sense. It preferably refers to all those elements which affect the generation, multiplication, function, use or value of the recombination system according to the invention, recombination construct according to the invention or cells or organisms comprising them. The following may be mentioned by way of example, but not by limitation, of the further functional elements.
Replication origins that ensure replication of the expression cassettes or vectors according to the invention in, for example, E. coli. Examples that may be mentioned are ORI (origin of DNA replication), the pBR322 on or the P15A on (Sambrook et al.: Molecular Cloning. A Laboratory Manual, 2.sup.nd ed. Cold Spring Harbor Laboratory Press, Cold Spring Harbor, N.Y., 1989).
Multiple cloning regions (MCS) enable and facilitate the insertion of one or more nucleic acid sequences.
Sequences which make possible homologous recombination or insertion into the genome of a host organism.
Elements, for example border sequences, which make possible the agrobacterium-mediated transfer in plant cells for the transfer and integration into the plant genome, such as, for example, the right or left border of the T-DNA or the vir region.

All of the above-mentioned expression cassettes or further functional elements may be located, as mentioned, between the homology or repeated sequences A and B of the nucleic acid sequence to be excised. However, they may also be located outside them. This is advantageous in particular in the case of border sequences.

The method of the invention is useful for obtaining plants from which genome a nucleic acid sequence has been excised. In addition to the “whole” plants or the “mature” plants, the invention also comprises progeny, propagation material (such as leaves, roots, seeds including embryo, endosperm, and seed coat, seedlings, fruit, pollen, shoots and the like), parts (organs, shoot vegetative organs/structures e.g. leaves, stems and tubers, roots, flowers, cuttings, and floral organs/structures, e.g. bracts, sepals, petals, stamens, carpels, anthers and ovules), tissues (e.g. vascular tissue, ground tissue, and the like), cells (e.g. guard cells, egg cells, trichomes and the like), cell cultures, and harvested material, derived from a plant which is obtained by the method according to the invention.

“Mature plants” are to be understood as meaning plants at any developmental stage beyond the seedling. “Seedling” is to be understood as meaning a young, immature plant in an early developmental stage. The “progeny” (or descendant) includes, inter alia, a clone, a seed, a fruit, selfed or hybrid progeny and descendants, and any propagule of any of these, such as cuttings and seed, which may be used in reproduction or propagation, sexual or asexual. Also encompassed by the invention is a plant that is a sexually or asexually propagated offspring, clone or descendant of such a plant, or any part or propagule of said plant, offspring, clone or descendant.

The term “cell” or “plant cell” as used herein refers to a single cell. The term “cells” refers to a population of cells. The population may be a pure population comprising one cell type. Likewise, the population may comprise more than one cell type. In the present invention, there is no limit on the number of cell types that a cell population may comprise. The cells may be synchronized or not synchronized. A plant cell within the meaning of this invention may be isolated (e.g., in suspension culture) or comprised in a plant tissue, plant organ or plant at any developmental stage.

The term “tissue” with respect to a plant (or “plant tissue”) means arrangement of multiple plant cells including differentiated and undifferentiated arrangements. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissue may be in planta, in organ culture, tissue culture, or cell culture.

Included within the scope of the invention are all genera and species of higher and lower plants of the plant kingdom. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The method according to the invention may preferably be used for the following plant families: Amaranthaceae, Brassicaceae, Carophyllaceae, Chenopodiaceae, Compositae, Cucurbitaceae, Labiatae, Leguminosae-Papilionoideae, Liliaceae, Linaceae, Malvaceae, Rosaceae, Saxifragaceae, Scrophulariaceae, Solanacea, Tetragoniacea and transgene combinations thereof.

Annual, perennial, monocotyledonous and dicotyledonous plants are preferred host organisms for the generation of transgenic plants. The use of the method according to the invention is furthermore advantageous in all ornamental plants, useful or ornamental trees, flowers, cut flowers, shrubs or turf. Plants which may be mentioned by way of example but not by limitation are angiosperms, bryophytes such as, for example, Hepaticae (hepaticas) and Musci (mosses); pteridophytes such as ferns, horsetail and clubmosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae.

Plants for the purposes of the invention comprise by way of example and not by way of limitation the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geraniums, Liliaceae such as drachaena, Moraceae such as ficus, Araceae such as philodendron and many others.

Flowering plants which may be mentioned by way of example but not by limitation are the families of the Leguminosae such as pea, alfalfa and soya; Gramineae such as rice, maize, wheat; Solanaceae such as tobacco and many others; the family of the Umbelliferae, particularly the genus Daucus (very particularly the species carota (carrot)) and Apium (very particularly the species graveo lens dulce (celery)) and many others; the family of the Solanacea, particularly the genus Lycopersicon, very particularly the species esculentum (tomato) and the genus Solanum, very particularly the species tuberosum (potato) and melongena (aubergine) and many others; and the genus Capsicum, very particularly the species annum (peppers) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli); and the genus Arabidopsis, very particularly the species thaliana and many others; the family of the Compositae, particularly the genus Lactuca, very particularly the species sativa (lettuce) and many others.

The transgenic plants according to the invention are selected in particular among monocotyledonous crop plants, such as, for example, cereals such as wheat, barley, sorghum and millet, rye, triticale, maize, rice or oats, and sugar cane. Further preferred are trees such as apple, pear, quince, plum, cherry, peach, nectarine, apricot, papaya, mango, and other woody species including coniferous and deciduous trees such as poplar, pine, sequoia, cedar, oak, etc. Especially preferred are Arabidopsis thaliana, Nicotiana tabacum, oilseed rape, soybean, corn (maize), wheat, linseed, potato and tagetes. The transgenic plants according to the invention are furthermore selected in particular from among dicotyledonous crop plants such as, for example, Brassicaceae such oilseed rape, cress, Arabidopsis, cabbages or canola, Leguminosae such as soya, alfalfa, peas, beans or peanut. Solanaceae such as potato, tobacco, tomato, aubergine or peppers, Asteraceae such as sunflower, Tagetes, lettuce or Calendula. Cucurbitaceae such as melon, pumpkin/squash or courgette, and linseed, cotton, hemp. Flax, red pepper, carrot, sugar beet and the various tree, nut and wine species.

Especially preferred for the method of the present invention are maize, Arabidopsis thaliana, Nicotiana tabacum and oilseed rape and all genera and species which are used as food or feedstuffs, such as the above-described cereal species, or which are suitable for the production of oils, such as oil crops (such as, for example, oilseed rape), nut species, soya, sunflower, pumpkin/squash and peanut.

Most preferred plants for the method of the present invention are maize, Arabidopsis, sorghum, rice, rapeseed, tobacco, wheat, rye, barley, oat, potato, tomato, sugar beet, pea, sugarcane, asparagus, soy, alfalfa, peanut, sunflower and pumpkin.

The transgenic plant line which is generated in step b) of the method of the invention, or cells or parts of this transgenic plant line, are used in step c) to perform a transient assay. This assay serves to analyze the functionality of the DSBI enzyme. The assay may avoid a time-consuming assessment of the activity of the DSBI enzyme and may allow instead a simple and rapid evaluation of the DSBI functionality.

In a preferred embodiment, the transient assay is an intrachromosomal homologous recombination (ICHR) assay. This assay is used to monitor the frequency of intra-chromosomal HR.

Preferably, the transient assay of step c) of the method according to the invention comprises a transient transformation of a reporter construct. This transformation can be performed by any of the transformation methods described above with respect to the transformation of the (DSBI encoding) construct, preferably by biolistic bombardment or PEG-mediated protoplast transfection.

Preferably, the reporter construct comprises a nucleic acid sequence to be excised, wherein the nucleic acid sequence comprises at least one recognition sequence which is specific for the enzyme of step a) of the method according to the invention for the site-directed induction of DNA double strand breaks, and wherein the nucleic acid sequence is bordered at both ends by a homology sequence which allows for homologous recombination.

By way of example, but not of limitation, one concept of those reporter constructs will be explained in the following. The transient assay system can employ, for example, a “recombination trap” consisting of overlapping parts of a recombinant gene, for example a beta-glucuronidase (GUS) gene, comprised within the reporter construct. The overlap between two fragments of the gene, e.g. the GUS gene, can be removed by HR, leading to restoration of the functional gene. Such HR events can be detected, e.g. in the case of the GUS gene as blue spots or sectors, when plants or plant parts or cells are histochemically stained.

Preferably, the reporter construct is selected from the group consisting of a GUS construct, a green fluorescent protein (GFP) construct, a chloramphenicol transferase construct, a luciferase construct, a beta-galactosidase construct, an R-locus gene product construct, a beta-lactamase construct, a xyl E gene product construct, an alpha amylase construct, a tyrosinase construct and an aequorin construct. The method of detection of those gene products is well known to the skilled person, and is described in the literature cited above.

After the transient assay of step c), the plant line which is generated in step b) of the method according to the invention is crossed with a plant line containing a nucleic acid sequence to be excised, wherein the nucleic acid sequence to be excised comprises at least one recognition sequence which is specific for the enzyme of step a) for the site-directed induction of DNA double strand breaks, and wherein the nucleic acid sequence to be excised is bordered at both sides by a repeated sequence which allows for a DNA repair mechanism (step d)).

“DNA repair” is a process by which a DNA damage, e.g. a double strand break, is identified and corrected. In the present invention, preferably this repair mechanism is homologous recombination (HR). Alternatively, the mechanism to repair the introduced double strand break may be nonhomologous end joining (NHEJ), precise ligation (PJ), or other mechanisms so that the sequence of interest is fully excised.

Non-homologous end joining (NHEJ) is a pathway that can be used to repair double-strand breaks in DNA. NHEJ is referred to as “non-homologous” because the break ends are directly ligated without the need for a homologous template, in contrast to homologous recombination, which requires a homologous sequence to guide repair. The term “non-homologous end joining” was coined in 1996 by Moore J. K. and Haber J. E. (Mol Cell Biol. 1996 May; 16(5):2164-73). NHEJ typically utilizes short homologous DNA sequences, termed microhomologies, to guide repair. Microhomologies in the single-stranded overhangs that are often present on the ends of double-strand breaks are used to promote restorative repair.

When these overhangs are compatible, NHEJ almost always repairs the break accurately, with no sequence loss. Imprecise repair leading to loss of nucleotides can also occur, but is much less common. A number of proteins are involved in NHEJ. The Ku heterodimer, consisting of Ku70 and Ku80, forms a complex with the DNA dependent protein kinase catalytic subunit (DNA-PKcs), which is present in mammals but absent in yeast. The DNA Ligase IV complex, consisting of the catalytic subunit DNA Ligase IV and its cofactor XRCC4, performs the ligation step of repair. The recently discovered protein XLF, also known as Cernunnos, is also required for NHEJ.

The term “crossing” means the mating between two plants (eventually representing two plant lines) wherein the two individual plants are of not-identical genetic background. In other words, the two parental types have different genetic constitution. For the cross-pollination with the plants comprising the nucleic acid to be excised, both the T₀ lines and the homozygous T₁ lines can be used.

The crossing may be performed via pollination. In general, pollination is the transfer of pollen from the male reproductive structure of a flower to the female reproductive structure of a flower. More precisely, the pollination is the transfer of pollen from an anther (of the stamen) to the stigma (of a pistil). The pollination, which represents the sexual reproduction in plants, results in fertilization and, usually, seed production. In general, pollination may occur on a single plant (self-pollination) or between different plants or plant varieties (cross-pollination).

In a preferred embodiment, the “nucleic acid sequence to be excised” comprises the T-DNA region or part thereof. Another possibility is that the nucleic acid sequence to be excised encodes a selection marker. This selection marker is preferably selected from the group consisting of negative selection markers, markers conferring resistance to a biocidal metabolic inhibitor, to an antibiotic or to a herbicide, positive selection markers and counter-selection markers.

Most preferably, the selection marker is selected from the group consisting of acetohydroxy acid synthase, D-serine deaminase, phosphinothricin acetyltransferase, 5-enolpyruvyl-shikimate-3-phosphate synthase, glyphosates degrading enzymes, dalapono inactivating dehalogenases, sulfonylurea- and imidazolinone-inactivating acetolactate synthases, bromoxynilo degrading nitrilases, Kanamycin- or G418-resistance genes, neomycin phosphotransferase, 2-desoxyglucose-6-phosphate phosphatase, hygromycin phosphor-transferase, dihydrofolate reductase, D-amino acid metabolizing enzyme, D-amino acid oxidase, gentamycin acetyl transferase, streptomycin phosphotransferase, aminoglycoside-3-adenyl transferase, bleomycin resistance determinant, isopentenyltransferase, beta-glucoronidase, mannose-6-phosphate isomerase, UDP-galactose-4-epimerase, cytosine deaminase, cytochrome P-450 enzymes, indoleacetic acid hydrolase, haloalkane dehalogenase and thymidine kinase.

Finally, after the crossing step d), an immature embryo conversion is performed (step e)). The “immature embryo conversion” is a process to recover a plant or a seedling from an immature embryo via in vitro conversion/germination of an immature embryo to a full seedling without callus formation. During this process, immature embryos can be placed onto the rooting medium and immature embryos are converted into seedlings. The process has been described, e.g., in “Green C. E. and Phillips R. L. Crop Science. Vol. 15 May-June 1975, pp 417-421”. The conversion is the ability of an embryo to germinate and, preferably, subsequently to develop into an established autotrophic plant. The germination is the initiation of physiological processes in an embryo, usually induced by the uptake of water and exposure to inductive environmental cues, resulting in meristematic growth (cell division and elongation), and ending with the complete development of cotyledons.

The term “immature embryo” refers to an embryo derived from a seed/kernel that has not fully developed and matured to a stage with the proper size, weight, and moisture content. These developing embryos possess ability to become a plant under suitable in vitro conditions.

Further preferred embodiments of the method of immature embryo conversion according to the invention will be described in the following by way of example.

After a successful pollination (self or cross-pollination), immature embryos are dissected from corn kernels about 10-20 days after pollination aseptically, placed onto an agar MS medium without the supplementation of phytohormones, and incubated under light at 20-27° C. The shoots and roots start to become visible after one day of incubation, and the full intact seedlings can be obtained in about 7 days. Each immature embryo becomes one intact seedling.

Alternatively to the “immature embryo conversion” of step e)—or subsequent hereto—a tissue culture regeneration through callus formation is performed. This step allows for recovering a transgenic plant containing a DNA double strand break inducing enzyme.

Preferably, the method of the present invention also comprises the identification of a single copy transgenic line following step b) or c). Preferably a single copy transgenic plant line is used for the further steps of the method of the invention. In the context of the present invention, the term “single copy” refers to the double strand break inducing enzyme.

The identification of a single copy transgenic line may be performed via any standard molecular technique that is known to the person skilled in the art. Preferably, the single copy identification is performed via quantitative PCR or Southern hybridization.

Quantitative PCR is a technique used to simultaneously quantify and amplify a specific part of a given DNA molecule. It is used to determine whether or not a specific sequence is present in the sample, and if it is present, the number of copies in the sample. The procedure follows the general pattern of polymerase chain reaction, but the DNA is quantified after each round of amplification. Two common methods of quantification are the use of fluorescent dyes that intercalate with double-strand DNA, and modified DNA oligonucleotide probes that fluoresce when hybridized with a complementary DNA. The techniques include SYBR Green quantitative PCR, Probe-based quantitative PCR and Quantitative Reverse Transcriptase PCR.

Details about PCR technologies may be found, e.g. in “PCR—Polymerase-Kettenreaktion. Das Methodenbuch” (Hans-Joachim Müller, Spektrum Akademischer Verlag, June 2001), “Molekularbiologische Diagnostik” (Frank Thiemann Hoppenstedt Publishing, 2002) or “Der Experimentator: Molekularbiologie/Genomics” (Cornel Mülhardt, Spektrum Akademischer Verlag, April 2006).

“Southern hybridization” or “Southern blot” is a method of enhancing the result of an agarose gel electrophoresis by marking specific DNA sequences. By way of a general example, but not of limitation, the method comprises the following steps:

1. DNA fragments are electrophoresed on a gel to separate DNA (e.g. deriving from a PCR) based on size.
2. If DNA is larger than 15 kb, prior to blotting, the gel may be treated with a dilute acid, such as dilute HCl which acts to depurinate the DNA fragments. This breaks the DNA into smaller pieces that will be able to complete the transfer more efficiently than larger fragments.
3. The gel from the DNA electrophoresis is treated with an alkaline solution (typically containing sodium hydroxide) to cause the double-stranded DNA to denature, separating it into single strands. Denaturation is necessary so that the DNA will stick to the membrane and be hybridized by the probe (see below).
4. A sheet of nitrocellulose (or, alternatively, nylon) membrane is placed on top of the gel. Pressure is applied evenly to the gel (either using suction, or by placing a stack of paper towels and a weight on top of the membrane and gel). This causes the DNA to move from the gel onto the membrane by capillary action, where it sticks.
5. The membrane is then baked (in the case of nitrocellulose) or exposed to ultraviolet radiation (nylon) to permanently crosslink the DNA to the membrane.
6. The membrane is now treated with a hybridization probe—an isolated DNA molecule with a specific sequence that pairs with the appropriate sequence (the appropriate sequence is the complementary sequence of what the restriction enzyme recognized). The probe DNA is labelled so that it can be detected, for example by incorporating radioactivity or tagging the molecule with a fluorescent or chromogenic dye. In some cases, the hybridization probe may be made from RNA, rather than DNA.
7. After hybridization, excess probe is washed from the membrane, and the pattern of hybridization is visualized on X-ray film, or equivalent technology, by autoradiography in the case of a radioactive or fluorescent probe, or by development of color on the membrane itself if a chromogenic detection is used.

The method was first described by Southern, E. M. (1975): “Detection of specific sequences among DNA fragments separated by gel electrophoresis”, J Mol Biol., 98:503-517. The skilled person know how to perform a Southern blot, for example according to the description in T. Maniatis, E. F. Fritsch and J. Sambrook, Molecular Cloning: A Laboratory Manual, Cold Spring Harbor Laboratory, Cold Spring Harbor, N.Y. (1989).

In another preferred embodiment of the present invention, the method comprises the analysis of the transgene expression level following step b) or c). The term “transgene” refers in this context to the double strand break inducing enzyme. In this context, the term “expression” includes transcription and translation. Preferably, a high or at least a medium expressing plant line is used for the further steps of the method of the invention. This step is performed either alternatively to the above-mentioned step of “identification of a single copy transgenic line” or—preferably—in addition to the latter, most preferably subsequent to the latter.

This analysis can be performed via any standard molecular technique that is known to the person skilled in the art. Preferably, the analysis of the transgene expression level is performed via RT-PCR or Northern hybridization.

Reverse transcription polymerase chain reaction (RT-PCR) is a technique for amplifying a defined piece of an RNA molecule. The RNA strand is first reverse transcribed into its DNA complement or complementary DNA, followed by amplification of the resulting DNA using polymerase chain reaction. This can either be a 1 or 2 step process. Polymerase chain reaction itself is the process used to amplify specific parts of a DNA molecule, via the temperature-mediated enzyme DNA polymerase.

In the first step of RT-PCR, called the “first strand reaction”, complementary DNA is made from a mRNA template using dNTPs and an RNA-dependent DNA polymerase, reverse transcriptase, through the process of reverse transcription. The above components are combined with a DNA primer in a reverse transcriptase buffer for an hour at about 37° C. After the reverse transcriptase reaction is complete, and complementary DNA has been generated from the original single-stranded mRNA, standard polymerase chain reaction, termed the “second strand reaction” is initiated.

1. A thermostable DNA polymerase and the upstream and downstream DNA primers are added.
2. The reaction is heated to temperatures above about 37° C. to facilitate sequence specific binding of DNA primers to the cDNA (copy DNA).
3. Further heating allow the thermostable DNA polymerase (“transcriptase”) to make double-stranded DNA from the primer bound cDNA.
4. The reaction is heated to approximately 95° C. to separate the two DNA strands.
5. The reaction is cooled enabling the primers to bind again and the cycle repeats.

After approximately 30 cycles, millions of copies of the sequence of interest are generated. The original RNA template is degraded by RNase H, leaving pure cDNA (plus spare primers). This process can be simplified into a single step process by the use of wax beads containing the required enzymes for the second stage of the process which are melted, releasing their contents, on heating for primer annealing in the second strand reaction. Northern blot techniques may be used to study the RNA's gene expression further.

The “Northern blot” or “Northern hybridization” is a technique used to study gene expression. It takes its name from the similarity of the procedure to the Southern blot procedure, used to study DNA, with the key difference that RNA, rather than DNA, is the substance being analyzed by electrophoresis and detection with a hybridization probe. A notable difference in the procedure (as compared with the Southern blot) is the addition of formaldehyde in the agarose gel, which acts as a denaturant. As in the Southern blot, the hybridization probe may be made from DNA or RNA.

The skilled person know how to perform a Northern blot, for example according to the description in T. Maniatis, E. F. Fritsch and J. Sambrook, Molecular Cloning: A Laboratory Manual, Cold Spring Harbor Laboratory, Cold Spring Harbor, N.Y. (1989).

In another preferred embodiment of the present invention, the method comprises the pollination of the transgenic plant line following step b) or c), wherein the pollination is either self-pollination or cross-pollination with a wild-type plant line. Self-pollination is preferred. Preferably, the lines that were previously identified to have a single copy DSBI transgene and/or to have a high or at least a medium DSBI expression are used for the pollination step.

After the self-pollination of the so-called “T₀ lines” (the lines arising from step b) of the method of the invention), the seeds obtained are called “T₁ seeds”.

The term “wild-type”, “natural” or of “natural origin” means with respect to an organism, polypeptide, or nucleic acid sequence, that said organism is naturally occurring or available in at least one naturally occurring organism which is not changed, mutated, or otherwise manipulated by man.

Preferably, the seeds and/or seedlings obtained through the pollination (the “T₁” seedlings) are analyzed for their zygosity. In the present case, this analysis determines the presence of the DSBI transgene and serves to identify the homozygous and the hemizygous plants. The term “homozygous” refers to two DNA sequences in the organism each located in the same genomic location, one on each homologous chromosome, “heterozygous”, or interchangeably hemizygous describes the presence of only a single copy of a gene (e.g. a transgene) on a single chromosome in an otherwise diploid (or polyploid) organism.

The zygosity analysis may be performed according to any standard molecular technique which is known to the person skilled in the art, for example via quantitative PCR, Southern hybridization and/or fluorescence in situ hybridization. The latter is defined as the use of a nucleic acid probe to detect and identify specific complementary sequences of DNA in chromosomes or RNA eukaryotic cells and tissues. The detection is performed via fluorescence, e.g. a probe coupled to a fluorescent dye.

In a further preferred embodiment, the homozygous lines which are identified after the zygosity analysis are selected for the crossing of step d) of the method according to the invention.

In another preferred embodiment of the present invention, the seeds and/or seedlings (called “F₁”) obtained by step e) of the method according to the invention are analyzed for DNA double strand break mediated homologous recombination.

Preferably, this homologous recombination analysis of the seeds and/or seedlings is determined by standard molecular techniques including PCR analysis, colorimetric or biochemical assays, or DNA sequencing. This analysis may be performed by the method as described for step c) of the method according to the invention. Alternatively or additionally, a PCR analysis may be performed. The selection of the primers depends on the nucleic acid sequence to be excised. The skilled person knows how to design an adequate PCR reaction, e.g. as described in T. Maniatis, E. F. Fritsch and J. Sambrook, Molecular Cloning: A Laboratory Manual, Cold Spring Harbor Laboratory, Cold Spring Harbor, N.Y. (1989).

The method as defined above can also be reversed, which means that it is directed to the reciprocal process. In this case, the transformation of step a) is performed with a construct encoding a nucleic acid sequence to be excised, the transient assay of step c) is performed in order to assess the functionality of the recognition sequence and the repeated sequence of the construct of step a), and the generated transgenic plant line is crossed with a plant line containing a DNA double strand break inducing enzyme. Therefore, the present invention is also directed to a method for excising a nucleic acid sequence from the genome of a plant or of a plant cell, comprising:
a) transforming a plant cell with a construct encoding a nucleic acid sequence to be excised, wherein the nucleic acid sequence to be excised comprises at least one recognition sequence which is specific for a DNA double strand break inducing enzyme for the site-directed induction of DNA double strand breaks, and wherein the nucleic acid sequence to be excised is bordered at both sides by a repeated sequence which allows for a DNA repair mechanism,
b) generating a transgenic plant line from the cell of step a),
c) performing a transient assay with the plant line of step b) or cells or parts thereof to analyze the functionality of the recognition sequence and the repeated sequence of the construct of step a),
d) crossing the plant line of step b) with a plant line containing a DNA double strand break inducing enzyme, and
e) performing either an immature embryo conversion or a tissue culture regeneration through callus formation.

All of the terms and procedures defined above apply in the same way to this reciprocal method, with the exception of the transient assay, which is preferably performed with a construct encoding a DNA double strand break inducing enzyme.

As an alternative to the crossing of step d), another approach of obtaining marker excision events is the “re-transforming” of the plant line—which was generated in step b) and analyzed in step c)—with a construct.

In the case that the transforming of step a) was performed with a construct encoding a DNA double strand break inducing enzyme, the re-transformation is performed with a construct encoding a nucleic acid sequence to be excised. Therefore, the present invention is directed to a method for excising a nucleic acid sequence from the genome of a plant or of a plant cell as defined above, wherein the crossing of step d) is replaced by a re-transforming of the plant line of step b) with a construct encoding a nucleic acid sequence to be excised, wherein the nucleic acid sequence to be excised comprises at least one recognition sequence which is specific for the enzyme of step a) for the site-directed induction of DNA double strand breaks, and wherein the nucleic acid sequence to be excised is bordered at both sides by a repeated sequence which allows for a DNA repair mechanism., wherein the nucleic acid sequence to be excised comprises at least one recognition sequence which is specific for the enzyme of step a) for the site-directed induction of DNA double strand breaks, and wherein the nucleic acid sequence to be excised is bordered at both sides by a repeated sequence which allows for a DNA repair mechanism.

In the “reciprocal” method, where the transforming of step a) was performed with a construct encoding a nucleic acid sequence to be excised, the re-transforming is performed with a construct encoding a DNA double strand break inducing enzyme. Therefore, the present invention is also directed to a method for excising a nucleic acid sequence from the genome of a plant or of a plant cell as defined above, wherein the crossing of step d) is replaced by a re-transforming of the plant line of step b) with a construct encoding a DNA double strand break inducing enzyme.

The re-transformation process of the transgenic plant line with the second construct may be performed according to the description above, i.e. by any of the above-mentioned transformation methods. The same applies to the structure of the second construct (genetic control elements, promoters, enhancers, polyadenylation signals, ribosome binding sites etc.), which may be designed according to the above description.

In this alternative method, the skilled person is aware that another (a second) selection marker system should preferably be used for the second transformation. If, for example, the selection marker system for the first construct was based on the “ahas” gene, the second selection marker system could be the “dsdA” gene comprised within the expression cassette of the construct encoding the nucleic acid sequence to be excised, or vice versa. Any other combination of any of the above-mentioned selection markers or any selection marker that is known to the skilled person can be used likewise.

Preferably, retransformation is performed on the transgenic plant containing the excision target DNA sequence, especially when the excision target DNA sequence is the first selection marker gene. For example, immature embryos derived from the first transgenic line containing the ahas selection marker gene that is the excision target is dissected, infected and co-cultivated with an agrobacterium strain containing a plasmid comprising of a second transformation cassette with the dsdA gene as the selection marker. The similar transformation steps are followed as if the first transgenic line is wildtype in reference to the second selectable marker gene. If the first transgenic line contains the first T-DNA with the ahas gene, for example, applying only D-serine for the dsdA gene can do the selection. Thus produced plants are analysed for the excision events.

The present invention is further directed to a plant obtained by the method according to the invention, or the progeny, propagation material, a part, tissue, cell or cell culture, derived from such a plant.

Finally, the invention is directed to the use of a plant or progeny, propagation material, part, tissue, cell or cell culture according to the invention as aliment, fodder or seeds or for the production of pharmaceuticals or chemicals.

The plants according to the invention may be consumed by humans or animals and may therefore also be used as food or feedstuffs, for example directly or following processing known in the art. Here, the deletion of, for example, resistances to antibiotics and/or herbicides, as are frequently introduced when generating the transgenic plants, makes sense for reasons of customer acceptance, but also product safety.

A further subject matter of the invention relates to the use of the above-described plants and structures derived from them, pharmaceuticals or chemicals, especially fine chemicals. Here again, the deletion of, for example, resistances to antibiotics and/or herbicides is advantageous for reasons of customer acceptance, but also product safety.

A “pharmaceutical” is understood as meaning a drug, a chemical drug, or a medicine, which is used in medical treatment, prevention or vaccination. “Fine chemicals” is understood as meaning enzymes, vitamins, amino acids, sugars, fatty acids, natural and synthetic flavors, aromas and colorants widely usable. Especially preferred is the production of tocopherols and tocotrienols, and of carotenoids. Culturing the transformed host organisms, and isolation from the host organisms or from the culture medium, is performed by methods known to the skilled worker. The production of pharmaceuticals such as, for example, antibodies or vaccines, is described by Hood E E, Jilka J M. (1999) Curr Opin Biotechnol. 10(4):382-386; Ma J K and Vine N D (1999) Curr Top Microbiol Immunol. 236:275-92).

Various further aspects and embodiments of the present invention will be apparent to those skilled in the art in view of the present disclosure. All documents mentioned in this specification are incorporated herein by reference. Certain aspects and embodiments of the invention will now be illustrated by way of example. It is to be understood that this invention is not limited to the particular methodology, protocols, cell lines, plant species or genera, constructs, and reagents described as such.

### REFERENCES

Nutter R C, Scheets K, Panganiban L C, Lommel S A. The complete nucleotide sequence of the maize chlorotic mottle virus genome. Nucleic Acids Res. 1989 Apr. 25; 17(8):3163-77.
Scheets K, Khosravi-Far R, Nutter R C. Transcripts of a maize chlorotic mottle virus cDNA clone replicate in maize protoplasts and infect maize plants. Virology. 1993 April; 193(2): 1006-9.
Scheets K. Maize chlorotic mottle machlomovirus and wheat streak mosaic rymovirus concentrations increase in the synergistic disease corn lethal necrosis. Virology. 1998 Mar. 1; 242(1):28-38.
Scheets K. Maize chlorotic mottle machlomovirus expresses its coat protein from a 1.47-kb subgenomic RNA and makes a 0.34-kb subgenomic RNA. Virology. 2000 Feb. 1; 267(1):90-101.
Czako M, Wenck A R, Marton L (1996) Negative selection markers for plants. In: Gresshoff P M (ed) Technology transfer of plant biotechnology. CRC press, Boca Raton, pp 67-93.
Jefferson R A (1987) Assaying chimeric genes in plants: the GUS gene fusion system. Plant Mol Biol Rep 5:387-405.
Schrott M (1995) Selectable marker and reporter genes. In: Potrukus I (ed) Gene transfer to plants. Springer, Berlin, Heidelberg, N.Y., pp 325-336.
Kozak, M (1987) An analysis of 5′-noncoding sequences from 699 vertebrate messenger RNAs Nucleic Acids Research, 15:20 8125-8148.

### EXAMPLES

### Materials and General Methods

Unless indicated otherwise, chemicals and reagents in the Examples were obtained from Sigma Chemical Company (St. Louis, Mo.), restriction endonucleases were from New England Biolabs (Beverly, Mass.) or Roche (Indianapolis, Ind.), oligonucleotides were synthesized by MWG Biotech Inc. (High Point, N.C.), and other modifying enzymes or kits regarding biochemicals and molecular biological assays were from Clontech (Palo Alto, Calif.), Pharmacia Biotech (Piscataway, N.J.), Promega Corporation (Madison, Wis.), or Stratagene (La Jolla, Calif.). Materials for cell culture media were obtained from Gibco/BRL (Gaithersburg, Md.) or DIFCO (Detroit, Mich.). The cloning steps carried out for the purposes of the present invention, such as, for example, restriction cleavages, agarose gel electrophoresis, purification of DNA fragments, transfer of nucleic acids to nitrocellulose and nylon membranes, linking DNA fragments, transformation of E. coli cells, growing bacteria, multiplying phages and sequence analysis of recombinant DNA, are carried out as described by Sambrook (1989). The sequencing of recombinant DNA molecules is carried out using ABI laser fluorescence DNA sequencer following the method of Sanger (Sanger 1977).

### 1. Double Strand Break (DSB) Mediated Homologous Recombination for Marker Excision in Maize

Homing endonuclease (HEN)-expression in plant cells can enhance intrachromosomal homologous recombination (ICHR). The expression level of the HEN transgene in the HEN plants plays an important role in influencing ICHR rate. In a conventional way, the ICHR assays are performed on tissues from progeny plants derived from crosses between HEN-expressing plants and plants containing the target sequences (FIG. 9A). Compared to Arabidopsis, as a plant species representing small size and short generation time, evaluation of this system in maize requires large amount of efforts, time, and resources including the greenhouse space as well as long generation time. To overcome these discrepancies and obtain successful marker excision in maize in an efficient and effective manner, the following methods were developed (FIGS. 9B and 9C).

In maize, the selected T0 plants (HEN lines as well as excision capable lines) were selfed to obtain T1 seeds. The T1 seedlings were examined for a zygocity test using TaqMan assays in order to identify homozygous lines. T0 lines showing medium to high levels of transgene expression were selected to increase efficiency of DSB-mediated HR and limit the number of plants to be used for crossing (FIG. 8). First, single copy lines were identified using a TaqMan semi-quantitative PCR assay. These single copy lines were tested in order to identify medium to high expression of the introduced gene (I-SceI or GU-US, FIGS. 1-4) at the mRNA levels using TaqMan real-time RT-PCR. Transgene expression levels were normalized to the expression level of an endogenous gene (single copy gene is preferable). Leaf tissues from some of the strongest I-SceI expressing lines were transformed with a plasmid encoding the GU-US gene for transient ICHR assays via biolistic transformation. Successful ICHR is indicated in this assay by the detection of blue GUS positive spots on bombarded leaf samples. This process can also be conducted by transferring I-SceI construct into leaf tissues of the transgenic GU-US lines. This transient assay system facilitated the identification of the HEN transgenic lines that were the best candidates for successful DSB-mediated HR. The selected transgenic lines were used for further experimentation to obtain DSB-mediated HR plants.

The selected HEN homozygous lines were cross pollinated with the plants comprising their complementary constructs (FIGS. 3 and 4); that is, the I-SceI lines were crossed with lines harboring the reporter constructs (GU-US) and the reporter lines were crossed with lines harboring the I-SceI constructs. As a control following the conventional method, the resulting progeny seeds comprising both I-SceI and GU-US constructs were analyzed for DSB-induced HR directly, and planted for evaluation of recombination in the whole plants (FIG. 9A). In the progeny seeds comprising both I-SceI and reporter constructs, DSB-mediated HR was observed strongly in endosperm and sporadically in scutellum, but not in the embryo axis under the control of constitutive promoters (e.g. maize ubiquitin promoter in combination with maize ubiquitin intron) for expression of I-SceI and excision capable gene or expression cassette. Since scutellum is the target tissue for maize transformation and regeneration via Agrobacterium, the immature embryos of the F1 plants were used for regeneration process to recover DSB-mediated HR plants via embryonic callus culture. This process allowed propagating and differentiating the tissues that showed DSB-mediated HR. Therefore even though DSB-mediated HR did not occur in the embryo axis, the probability of identifying DSB-mediated HR lines was significantly increased (FIG. 9B). In addition, the I-SceI constructs were re-transformed into the immature embryos of the homozygous GU-US lines to improve DSB-mediated HR, since this process will go through regeneration process (FIG. 9C). Table 1 summarizes the basic features and the timeframes of these new approaches.

TABLE 1
Summary of three approaches for obtaining marker-
free event with HEN I-SceI gene in corn.

| | Timeline² to obtain the | |
|---|---|---|
| | excision target marker- | |
| Approaches¹ | free evens | Features exemplified in this invention |
| A. | Minimum 19 months | Low chance of obtaining marker excision event, |
| | | and/or requirement for large scale seed screening |
| | | (due to low frequency of full excision in a seed). |
| B. | 8 month | Increased chance of obtaining marker-excision |
| | | event (due to the potential of recovering excision |
| | | event at a single cell level, and the tissue culture |
| | | process promotes the recombination activities). |
| C. | 9 months | Increased chance of obtaining marker-excision |
| | | event (due to the high potential of obtaining the |
| | | fully excised event based on single cell |
| | | transformation) |

¹Approaches based on FIG. 9.
²A generation time is needed to segregate the I-SceI gene from the final excision-target marker-free event.

As described above, when a strong, constitutive, ubiquitous promoter was used to express the HEN gene and GU-US, no DSB-mediated HR, (indicated by blue spots) was detected in embryo axis in maize upon analysis of mature kernel. To achieve DSB-mediated HR in embryo, the super promoter was chosen, since this promoter in maize shows strong expression in the whole embryo (scutellum and embryo axis) during germination, calli (including embryogenic calli) during regeneration. The expression levels in these tissues can be enhanced by addition of intron-mediated enhancement (IME)-conferring intron between the super promoter and I-SceI gene.

The I-SceI homing endonuclease gene sequence was optimized to improve expression and mRNA stability. The sequence was optimized using a 50% mix of maize and soybean preferred codons. RNA instability motifs, codon repeats, cryptic splice sites, unwanted restriction sites and mRNA secondary structures were identified and removed to arrive at the final optimized sequence. The synthetic sequence was synthesized by Entelechon GmbH, Regensburg, Germany. The gene was synthesized with adenine at ±3 (+1 nucleotide for adenine of ATG as a translational start codon) Kozak consensus (Kozak, 1987), selected restriction sites and Gateway attachment regions (FIG. 22).

| | |
|---|---|
| SEQ ID NO: 26 | coding sequence of codon optimized I-SceI |
| SEQ ID NO: 27 | optimized I-SceI CDS with attachment regions |

With the method according to the invention, fully recombined maize was generated in the T₁ generation for both the excision of an entire reporter gene cassette and for recombination of the GU/US reporter at the rate of 1.8-9.6% and at 5.4-16.4% efficiency at 95% confidence, respectively.

The following flowchart is provided to give a brief and simplified overview over the examples described in the following. It is not to be understood as limiting.

### A. Agrobacterium-Mediated Transformation of Maize Immature Embryos

a) with I-Sce I construct (selectable marker cassette: ahas)
b) with GUS pseudo marker excision construct (marker: ahas)
c) with GU-US reporter construct (marker: ahas)
Agrobacterium inoculation, co-cultivation, selection, and plant regeneration
1. Inoculating the immature embryos with agrobacterium cell suspension;
2. Performing co-cultivation of immature embryos and agrobacterium on the medium without antibiotics and selection agents;
3. Transferring cultures to the recovery medium that contains antibiotics, but without the selection agents; Embryogenic callus production initiates from the scutellum at this stage;
4. Selecting transgenic embryogenic calli on medium with selection agent;
5. Recovering plantlets on regeneration medium with the selection agent;
6. Transferring young T₀ seedlings to rooting medium with the selection agents;
7. Performing TaqMan copy number analysis, and identify single copy events;
8. Transferring rooted, TaqMan-positive, single copy plants to soil;
9. Determining the transgene expression level via RT-PCR;
10. T₀ lines are self-pollinated to obtain T₁ seeds (harvest and storage). T₁ seedlings are examined for homozygosity via TaqMan assay

### B. Semi-Transient Assay System for the Proof of Concept on DSB-Mediated HR:

GU-US plasmid is introduced in the I-Sce I expressing Maize lines
a) in leaf tissue via particle gun or
b) in protoplasts via PEG-mediated transformation
determination of the transient expression levels of the reporter gene=transient ICHR assay (intrachromosomal homologous recombination) detection of the blue=positive spots→identification of the HEN transgenic T₀ lines that are the best candidates

### C. Cross Pollination

of a selected homozygous transgenic I-Sce I line with a line containing a selection marker bordered by excision sites (or with a GU-US or a GUS construct):
I-Sce I×GU-US=F1 progenies (embryos/seeds) are obtained

### D. Immature Embryo Conversion

(a process to recover a plant from an immature embryo via in vitro conversion/germination of an immature embryo to a full seedling without callus formation. F1 immature embryos are placed onto the rooting medium without auxin 2,4-D, and immature embryos are then converted into seedlings.)
(to avoid the time-consuming screening of F1 plants which are derived from the mature seeds)
F1 immature embryos are placed on rooting medium containing the selection agent against the selection marker linked to the I-Sce I gene→seedlings are recovered

### E. Plant Regeneration Via Embryogenic Calli

This is a process for recovering potentially marker-excised plants through a tissue culture process via callus formation. F1 immature embryos are cultured on the recovery (to promote callus formation), the selection (to promote embryogenic callus formation and to select cells containing integrated T-DNA), regeneration (to convert mature calli to plantlets) and then the rooting (to recover full seedlings) media. The major advantage of this process is to recover a F1 plant from a single cell, hence to separate/select a marker-excised cell line.

### F. Re-Transformation of a Transgenic Line

This is a technique applied to marker excision in addition to the routine crossing between two transgenic lines (the marker-excision target line and the HEN line, for example). To conduct re-transformation, immature embryos of the transgenic line A (e.g. the excision target line with first selection marker gene in the T-DNA) is transformed again with the agrobacterium strain containing HEN gene in its T-DNA, or immature embryos of the transgenic line B (e.g. the HEN line) can be transformed again with the agrobacterium strain containing the excision target T-DNA (or vice versa). The marker-excised seedlings can be recovered from the re-transformation process.

### G. Plant Analysis for the DSB-Mediated HR or Marker Excision

molecular screening for identification of the marker-free (=full marker-excision) plants
a) GUS histochemical staining assay of leaf and kernels
b) PCR analysis for marker excision

### 1.1 Vector Construction

### 1.1.1 Homing Endonuclease (I-SceI) Constructs

I-SceI was PCR amplified from vector pCB586-4 using primers 1 and 2.

| SEQ ID NO: 7: | Primer 1 (AscI-ATG-I-SceI 5′): |
|---|---|
| 5′-AGGCGCGCCATGAAAAACATCAAAAAAAACCA | |
| SEQ ID NO: 8: | Primer 2 (SbfI-TAA-I-SceI 3′): |

5′-GCTCCTGCAGGTTATTTCAGGAAAGTTTC

The resulting PCR product was digested with AscI and SbfI and cloned into AscI and SbfI digested vector pLM065 to produce vector pJB010, which comprises an expression cassette wherein the maize Ubiquitin promoter drives the expression of I-SceI.

Vector pCER 040b is a binary expression vector where the maize Ubiquitin promoter drives the expression of I-SceI, and was generated by ligation of the T4 DNA polymerase filled-in PmeI-XbaI fragment of pJB010 into T4 DNA Polymerase filled in AscI-PacI digested pEG085.

Vector pCER 041 is a binary expression vector where the sugarcane bacilliform virus (ScBV) promoter drives the expression of I-SceI, and was generated by ligation of the T4 DNA polymerase filled-in PacI-AscI fragment of pJB010 into T4 DNA Polymerase filled in EcoRV-AscI digested pEG085.

### 1.1.2 GU-US Construct

Vector pCB642-2 encodes an expression cassette wherein the GUS ORF comprises an internal duplication of 610 base pairs, with an I-SceI recognition site situated between the duplicated regions (GU-US). The fragment encoding the GU-US and NOS terminator was isolated from pCB642-2 by digestion with HindIII and SpeI, and was cloned into HindIII and SpeI digested pBluescript to generate pJB028.

The binary GU/US expression vector pJB034 was generated by ligation of the T4 DNA Polymerase filled in SpeI—XhoI fragment from pJB028 into T4 DNA Polymerase filled in EcoRV—AscI digested pEG085.

### 1.1.3 Pseudo-Marker Excision Construct

Vector pJB035 was generated by ligating the AscI fragment of pBPSMM247b comprising the GUS expression cassette (p-ScBV:GUS:t-NOS) into AscI-digested pUC001.

In order to introduce I-SceI sites flanking the GUS expression cassette in pJB035, oligos encoding the recognition sequence were generated Annealing of oligos 3 and 4 resulted in a double-stranded I-SceI recognition site with a Sail-compatible overhang on one end and an XbaI-compatible overhang on the other.

| | | |
|---|---|---|
| SEQ ID NO: 9: | Oligo 3 (SalI I-SceI): | 5′-TCGATAGGGATAACAGGGTAAT |
| SEQ ID NO: 10: | Oligo 4 (XbaI-I-SceI): | 5′-CTAGATTACCCTGTTATCCCTA |

An I-SceI site was added downstream of the GUS expression cassette by digesting pJB035 with SalI and XbaI and ligating in annealed oligos 3 and 4, thereby generating pJB036.

Oligos 5 and 6 were generated in order to produce a double stranded I-SceI sequence with Pad compatible ends.

| | | |
|---|---|---|
| SEQ ID NO: 11: | Oligo 5 (PacI-I-SceI 5′): | 5′-TAGGGATAACAGGGTAAT |
| SEQ ID NO: 12: | Oligo 6 (PacI-I-SceI 3′): | 5′-TACCCTGTTATCCCTAAT |

Annealed oligos 5 and 6 were ligated into PacI-digested pJB036 to generate pJB037.

The final pseudo-marker excision vector required a duplicated DNA sequence flanking the I-SceI sites in order to serve as a target sequence for homologous recombination (HR target). For this purpose, a portion of the maize AHAS terminator was duplicated. The region was excised via the 850 by EcoRI KpnI fragment from vector pEG085. This EcoRI-KpnI fragment was cloned into EcoRI and KpnI digested pJB037 to generate pJB038, which comprises the [I-SceI:p-ScBV:GUS:t-NOS:I-SceI:HR target] pseudo-marker cassette.

The pseudo-marker binary vector was generated by ligation of the 5.2 Kb HpaI-PmeI fragment from pJB038 into T4 DNA Polymerase filled in Pad AscI digested pEG085, to generate pJB039.

### 1.2 Agrobacterium-Mediated Corn Transformation and Regeneration

### 1.2.1 Plant Tissue Culture and Bacterial Culture Media

Unless indicated otherwise, chemicals and reagents in the Examples were obtained from Sigma Chemical Company (St. Louis, Mo.). Materials for cell culture media were obtained from Gibco/BRL (Gaithersburg, Md.) or DIFCO (Detroit, Mich.). The cloning steps carried out for the purposes of the present invention, such as, for example, transformation of E. coli cells, growing bacteria, multiplying phages and sequence analysis of recombinant DNA, are carried out as described by Sambrook (1989). The following examples are offered by way of illustration and not by way of limitation.

### Media Recipes

Imazethapyr (Pursuit) stock solution (1 mM) is prepared by dissolving 28.9 mg of Pursuit into 100 ml of DMSO (Sigma), and stored at 4° C. in the dark. Acetosyringone stock is prepared as 200 mM solution in DMSO and stored at ±20° C. D-serine stock solution is prepared in the double distilled water, filter-sterilized and store at 4° C.

TABLE 2
Maize YP Media (for growing Agrobacterium)

| | Supplier/ | Final |
|---|---|---|
| Media Components | Catalog # | Concentration |
| Yeast extract | Sigma Y1626 | 5 g/L |
| Peptone (from meat) | EM V298413 | 10 g/L |
| NaCl | Sigma S5886 | 5 g/L |

Adjust pH to 6.8 with 1 M NaOH. For solid medium add 3 g agar (EM Science) per 250 mL bottle. Aliquot 100 mL media to each 250 mL bottle, autoclave, let cool and solidify in bottles. For plate preparation, medium in bottle is melted in microwave oven, and the bottle is placed in water bath and cool to 55° C. When cooled, add spectinomycin (Sigma S-4014) to a final concentration of 50 mg/L mix well and pour the plates.

TABLE 3
Maize LS-inf Medium

| | Supplier/ | Final | |
|---|---|---|---|
| Media Components | Catalog # | Concentration | |
| MS (Murashige and Skoog basal media) | Sigma M-5524 | 4.3 | g/L |
| Vitamin assay casamino acids (Difco) | Difco vitamin | 1.0 | g/L |
| | assay | | |
| Glucose | Sigma G7528 | 36 | g/L |
| Sucrose | Sigma S5391 | 68.5 | g/L |
| 2,4-D (stock at 0.5 mg/mL) | Sigma D7299 | 1.5 | mg/L |
| Nicotinic acid (stock 0.5 mg/mL) sterile | Sigma N4126 | 0.5 | mg/L |
| Pyridoxine HCl (0.5 mg/mL) sterile | Sigma P8666 | 0.5 | mg/L |
| Thiamine HCl (1.0 mg/mL) sterile | Sigma T4625 | 1.0 | mg/L |
| Myo-inositol (100 mg/mL) sterile | Sigma I5125 | 100 | mg/L |

Adjust pH to 5.2 with 1 M HCl, filter sterilize, dispense in 100 mL aliquots, add acetosyringone (100 μM) to the medium right before used for Agrobacterium infection (50 μL to 100 mL media-200 mM stock).

TABLE 4
Maize 1.5LSAs Medium (for co-cultivation)

| | Supplier/ | Final | |
|---|---|---|---|
| Media Components | Catalog # | Conc. | |
| MS (Murashige and Skoog basal media) | Sigma M-5524 | 4.3 | g/L |
| Glucose | Sigma G7528 | 10 | g/L |
| Sucrose | Sigma S5391 | 20 | g/L |
| 2,4-D (stock at 0.5 mg/mL) | Sigma D7299 | 1.5 | mg/L |
| Nicotinic acid (stock 0.5 mg/mL) sterile | Sigma N4126 | 0.5 | mg/L |
| Pyridoxine HCl (0.5 mg/mL) sterile | Sigma P8666 | 0.5 | mg/L |
| Thiamine HCl (1.0 mg/mL) sterile | Sigma T4625 | 1.0 | mg/L |
| Myo-inositol (100 mg/mL) sterile | Sigma I5125 | 100 | mg/L |
| L-proline (stock 350 mg/mL) | Sigma P5607 | 700 | mg/L |
| MES (stock 250 mg/mL) | Sigma M3671 | 500 | mg/L |

Adjust pH media to 5.8 with 1 M NaOH. Weigh 4 g Sigma Purified Agar per bottle (8 g/L) and dispense 500 mL media per bottle, autoclave. When cooled add AgNO3 (stock at 15 mM) to a final concentration of 15 μM and L-cysteine (stock at 150 mg/ml) to a final concentration 300 mg/l. Pour into 100×20 mm Petri plates. Medium containing acetosyringone should be used freshly without long-term storage.

TABLE 5
Maize Recovery Medium: IM medium

| | Supplier/ | Final | |
|---|---|---|---|
| Media Components | Catalog # | Conc. | |
| MS(Murashige and Skoog basal media) | Sigma M-5524 | 4.3 | g/L |
| Sucrose | Sigma S5391 | 30 | g/L |
| 2,4D(stock 0.5 mg · ml) | Sigma D7299 | 1.5 | mg/mL |
| Casein hydrolysate | V919638 | 100 | mg/L |
| Proline | Sigma P5607 | 2.9 | g/L |

Measure ˜¾ of the total volume ddH₂O desired, add sucrose and salts, and dissolve under stirring. After all ingredients are dissolved, adjust to final volume with ddH₂O and to pH 5.8 using 1M KOH. Aliquot 500 mls of liquid medium into a 1 L bottle with 0.9 g gelrite, autoclave for 20 minutes (liquid cycle). After autoclaving place bottles into a water-bath to cool to 55° C. and add MS Vitamins (to a final concentration of 1.0 mg/mL), silver nitrate (to final concentration of 15 μM) and Timentin (to final concentration of 150 mg/L). Pour media into 100×20 mm petri plates and allow media to remain in the laminar hood overnight to prevent excess condensation.

TABLE 6a
Selection Media

| | Supplier/ | Final | |
|---|---|---|---|
| Media Components | Catalog # | Concentration | |
| MS (Murashige and Skoog basal media) | Sigma M-5524 | 4.3 | g/L |
| Sucrose | Sigma S5391 | 20 | g/L |
| 2,4-D (stock at 2.0 mg/mL) | Sigma D7299 | 0.5 | mg/L |
| Nicotinic acid (stock 0.5 mg/mL) sterile | Sigma N4126 | 0.5 | mg/L |
| Pyridoxine HCl (0.5 mg/mL) sterile | Sigma P8666 | 0.5 | mg/L |
| Thiamine HCl (1.0 mg/mL) sterile | Sigma T4625 | 1.0 | mg/L |
| Myo-inositol (100 mg/mL) sterile | Sigma I5125 | 100 | mg/L |
| L-proline (stock 350 mg/mL) | Sigma P5607 | 700 | mg/L |
| MES (stock 250 mg/mL) | Sigma M3671 | 500 | mg/L |

Adjust pH of media to pH 5.8 with 1 M NaOH. Add Sigma Purified Agar (8 g/L), dispense 500 mL medium per 1 L bottle, autoclave, when cooled add (Table 6b):

| Medium type | Post autoclaving components | Supplier/Catalog # | Final Concentration | |
|---|---|---|---|---|
| Selection with | Timentin (stock at 200 mg/ml) | Bellamy DS | 150 | mg/L |
| Pursuit | Pursuit (stock at 1 mM) | AC263, 499 | 500 | nM |
| | Picloram (2 mg/mL) | Sigma Z0876 | 2 | mg/L |
| Selection with | Timentin (stock at 200 mg/mL) | Bellamy DS | 150 | mg/L |
| D-Serine | D-Serine (Stock at 1M) | AlfaAesar A11353 | 10 | mM |
| | Picloram (2 mg/mL) | Sigma Z0876 | 2 | mg/L |

TABLE 7a
Maize Regeneration Media

| | Supplier/ | Final | |
|---|---|---|---|
| Media Components | Catalog # | Concentration | |
| MS (Murashige and Skoog basal media) | Sigma M-5524 | 4.3 | g/L |
| Sucrose | Sigma S5391 | 20 | g/L |
| Nicotinic acid (stock 0.5 mg/mL) sterile | Sigma N4126 | 0.5 | mg/L |
| Pyridoxine HCl (0.5 mg/mL) sterile | Sigma P8666 | 0.5 | mg/L |
| Thiamine HCl (1.0 mg/mL) sterile | Sigma T4625 | 1.0 | mg/L |
| Myo-inositol (100 mg/mL) sterile | Sigma I5125 | 100 | mg/L |
| L-proline (stock 350 mg/mL) | Sigma P5607 | 700 | mg/L |
| MES (stock 250 mg/mL) | Sigma M3671 | 500 | mg/L |

Adjust pH media to 5.8 with 1 M NaOH. Weigh 4 g Sigma Purified Agar (Sigma A7921) per bottle (8 g/L). Dispense 500 mL media per bottle, autoclave and let solidify in bottles. For use, microwave to melt media, when cooled, add (Table 7b):

| | Post autoclaving | | | |
|---|---|---|---|---|
| Type of media | components | Supplier/Catalog # | Final Concentration | |
| Regeneration medium | Timentin (200 mg/mL) | Bellamy DS | 150 | mg/L |
| with Pursuit | Pursuit (stock at 1 mM) | AC263, 499 | 500 | nM |
| | Zeatin (stock at 5 mg/mL) | Sigma Z0876 | 2.5 | mg/L |
| Regeneration medium | Timentin (200 mg/mL) | Bellamy DS | 150 | mg/: |
| with D-Serine | D-Serine (stock at 1 mM) | AlfaAesar A11353 | 15 | mM |
| | Zeatin (stock at 5 mg/mL) | Sigma Z0876 | 2.5 | mg/L |

Pour into 100×20 mm Petri plates

TABLE 8
Maize Rooting Media

| | Supplier/ | Final | |
|---|---|---|---|
| Media Components | Catalog # | Concentration | |
| ½ MS (Murashige and Skoog basal | Sigma M-5524 | 2.15 | g/L |
| media) | | | |
| Sucrose | Sigma S5391 | 20 | g/L |
| Nicotinic acid (stock 0.5 mg/mL) sterile | Sigma N4126 | 0.5 | mg/L |
| Pyridoxine HCl (0.5 mg/mL) sterile | Sigma P8666 | 0.5 | mg/L |
| Thiamine HCl (1.0 mg/mL) sterile | Sigma T4625 | 1.0 | mg/L |
| Myo-inositol (100 mg/mL) sterile | Sigma I5125 | 100 | mg/L |
| L-proline (stock 350 mg/mL) | Sigma P5607 | 700 | mg/L |
| MES (stock 250 mg/mL) | Sigma M3671 | 500 | mg/L |

Adjust pH of media to pH 5.8 with 1 M NaOH, add 1 g Gelrite per bottle (2 g/L), dispense 500 mL media per bottle, autoclave, pour into disposable Phyatrays after adding the selection agents.

| | Post autoclaving | | | |
|---|---|---|---|---|
| Type of media | components | Supplier/Catalog # | Final Concentration | |
| Rooting medium with | Timentin (200 mg/ml) | Bellamy DS | 150 | mg/L |
| Pursuit | Pursuit (stock at 1 mM) | AC263, 499 | 500 | nM |
| | Zeatin (stock at 5 mg/mL) | Sigma Z0876 | 2.5 | mg/L |
| Rooting medium with | Timentin (200 mg/mL) | Bellamy DS | 150 | mg/L |
| D-Serine | D-Serine (stock at 1 mM) | AlfaAesar A11353 | 10 | mM |
| | Zeatin (stock at 5 mg/mL) | Sigma Z0876 | 2.5 | mg/L |

### 1.3 Preparation of Donor Plants for Transformation Experiments

1.3.1 Deposit under the Budapest Treaty

A deposit was made under the Budapest Treaty for the following material:

1. Seed of Zea mays line BPS553; Patent Deposit Designation PTA-6170.
2. Seed of Zea mays line BPS631; Patent Deposit Designation PTA-6171.

The deposit was made with the American Type Culture Collection (ATCC), Manassas, Va. 20110-2209 USA on Aug. 26, 2004.

### 1.3.2 Preparation of Hybrid Donor Plants

The following Zea mays inbred lines are employed for the following steps:

1. HiIIA: HiII parent A; deposit No.: T0940A, Maize Genetics and Genomics Database), available from Maize Genetics Cooperation—Stock Center USDA/ARS & Crop Sci/UIUC, S-123 Turner Hall, 1102 S. Goodwin Avenue, Urbana Ill. USA 61801-4798; http://www.maizegdb.org/stock.php.

### 2. A188: Agronomy & Plant Genetics, 411 Borlaug Hall, Univ of Minnesota, Saint Paul Minn. 55108.

### 3. BPS533 (ATCC Patent Deposit Designation PTA-6170).

### 4. BPS631 (ATCC Patent Deposit Designation PTA-6171).

F1 seeds of corn genotype HiIIAxA188 are produced by crossing HiIIA (female parent) with inbred line A188 (male), and planted in the greenhouse as pollen donor. F2 seeds of (HiIIAxA188) are produced by self-pollination of F1 (HiIIAxA188) plants either in the greenhouse or in the field, and planted in the greenhouse as the pollen donor. Hybrid immature embryos of BPS553x(HiIIAxA188) or BPS631x(HiIIAxA188) are produced using inbred line BPS553 (ATCC Patent Deposit Designation PTA-6170) or BPS631 (ATCC Patent Deposit Designation PTA-6171) as the female parents, and either F1 or F2 (HiIIAxA188) plants as the male parent in the greenhouse.

Seeds are sowed in pots containing Metromix. Once the seeds become germinated and rooted, one seedling/pot is maintained for immature embryo production, and the second seedling is discarded; Alternatively seeds are started in a 4×4 inch pots, and seedlings are transplanted to 10-inch pots two weeks after sowing the seeds. Approximately one tablespoon of Osmocote 14-14-14 (a type of slow releasing fertilizer) is added to the surface of each pot. The temperature in the greenhouse is maintained at 24° C. night and 28° C. day. Watering is done automatically, but is supplemented daily manually as needed. Twice a week, the plants are watered with a 1:15 dilution of Peters 20-20-20 fertilizer.

### 1.3.3 Preparation of Inbred Donor Plants

Seeds of inbred lines BPS553 or BPS631 are sown either directly in 4-inch pots, and the seedlings are transplanted to 10-inch pots two weeks after sowing the seeds. Alternatively, seeds are directly sown into 10-inch pots. Self- or sib-pollination is performed. The growing conditions are same as above for the hybrid line.

### 1.3.4 Hand-Pollination

Every corn plant is monitored for ear shoots, and when appeared, they are covered with a small white ear shoot bag (Lawson). Once the ear shoots have started to produce silks, the silks are cut and covered again with the ear shoot bag. The tassel of the same plant is bagged with a brown paper bag (providing that the tassel has entered anthesis). The next morning, the tassel is shaken to remove pollen and anthers into the bag. The bag is then removed and pollen is shaken over the silks of the ear shoot. Pollinating is done between 8 and 10 a.m. in the morning. Secure the brown paper bag over the ear shoot and around the corn stalk. After pollination, the tassel is removed from the plant to reduce pollen (allergens to many people) in the greenhouse.

To ensure synchronized pollinations for the same genotypes, and hence to avoid weekend harvesting/transformation, ear shoots of those early flowering plants are cut back again. A group of plants, e.g. >5 to 10 plants are then pollinated on the same day. However, this practice is dependent on the quality/quantity of pollens on a plant. Sib-pollination is needed for the inbred lines. For instance either BPS553 or BPS631 can be either selfed or sib-pollinated between the same genotype).

### 1.3.5 Harvest and Pre-Treat Ears

Ears from corn plants (the first ear that comes out is the best) are harvested 8 to 14 (average 10) days after pollination (DAP). Timing of harvest varies depending on growth conditions and maize variety. The size of immature embryos is a good indication of their stage of development. The optimal length of immature embryos for transformation is about 1 to 1.5 mm, including the length of the scutellum. The embryo should be translucent, not opaque. If the ear is ready, but can not be used for transformation that day, the ear can be harvested, put in the pollination bag, and stored in a plastic bag in 4° C. fridge for 1 to 3 days.

### 1.4 Agrobacterium Mediated Transformation

### 1.4.1 Preparation of Agrobacterium

Agrobacterium glycerol stock is stored at ±80° C. Inoculums of Agrobacterium are streaked from glycerol stocks onto YP agar medium (A-1) containing appropriate antibiotics (e.g. 50 mg/L spectinomycin and/or 10 mg/L tetracycline, or 100 mg/l kanamycin). The bacterial cultures are incubated in the dark at 28° C. for 1 to 3 days, or until single colonies are visible. The obtained plate can be stored at 4° C. for 1 month and used as a master plate to streak out fresh cells. Fresh cells should be streaked onto YP agar with the appropriate antibiotic from a single colony on the master plate, at least 2 days in advance of transformation. These bacterial cultures can be incubated in the dark at 28° C. for 1 to 3 days.

Alternatively frozen Agrobacterium stock can be prepared by streaking Agrobacterium cells from frozen stock to a plate B-YP-002 (YP+50 mg/L spectinomycin+10 mg/L tetracycline), and growing at 28° C. for 2 to 3 days. Save it as master plate and store at 4 C for up to a month. From the master plate, streak a loop of agro cells to a flask containing 25 mL liquid B-YP-000 medium supplemented with 50 mg/L Spectinomycin+10 mg/l tetracycline. Grow on a shaker set at 300 rpm and 28° C. 2 to 3 days. Prepare frozen agro stock by mixing 1 part of the above agro culture with 1 part of sterile 30% glycerol. Vortex to mix well and dispense 10 μL the Agrobacterium/glycerol mixture to a 50 μL Eppendorf tube. Store at ±80° C.

One loop full (2 mm in diameter) of bacterial culture is suspended in 1.0 to 1.8 mL LS-inf medium supplemented with 200 nM acetosyringone. This yields a bacterial suspension with approximate optical density (OD₆₀₀) between 0.5 to 2.0. Vortex for 0.5 to 3 hours. Vortexing is performed by fixing (e.g. with tape) the microfuge tube horizontally (instead of vertically) on the platform of a vortexer to ensure better disperse Agrobacterium cells into the solution. Mix 100 μL of Agrobacterium cell suspension with 900 uL of LS-inf solution in a curvet, and measure OD₆₀₀. Adjust OD of original Agrobacterium solution to 0.6 to 2.0 with LS-Inf (with 100 nM acetosyringone) solution. The Agrobacterium suspension must be vortexed in the LS-inf+acetosyringone media for at least 0.5 to 3 hours prior to infection. Prepare this suspension before starting harvesting embryos.

Alternatively Agrobacterium suspensions for corn transformation can be prepared as follows: Two days before transformation, from −80° C. stock, streak Agrobacteria from one tube to a plate containing B-YP-002 (solidified YP+50 mg/L spectinomycin+10 mg/l tetracycline) and grow at 28° C. in the dark for two days. About 1 to 4 hrs before transformation, place one scoop of bacterial cells to 1.5 mL M-LS-002 medium (LS-inf+200 μM acetosyrigone) in a 2 mL Eppendorf tube. Vortex the tube to dispense the bacterial cells to solution and shake the tube at 1000 rpm for 1 to 4 hrs. The OD₆₀₀ should be in the range of 0.6 to 1.0 or about 10⁸ cfu/mL.

For the purpose of the following examples Agrobacterium tumefaciens strain LBA4404 or disarmed Agrobacterium strain K599 (NCPPB 2659)) transformed with binary vector plasmid pBPSMM232 were employed. pBPSMM232 contains the ahas gene (as selection marker) and the gus reporter gene.

### 1.4.2 Surface Sterilization of Corn Ear and Isolation of Immature Embryos

The ears are harvested from the greenhouse 8 to 12 days after pollination. All husk and silks are removed and ears are transported in the brown pollination bag back to the tissue culture lab. The cob is moved into the sterile hood. A large pair of forceps is inserted into the basal end of the ear and the forceps are used as a handle for handling the cob. Optionally, when insects/fungus are present on the ear, the ear should be first sterilized with 20% commercial bleach for 10 min (alternatively 30% Clorox solution for 15 min), and then rinsed with sterilized water three times. While holding the cob by the forceps, the ear is completely sprayed with 70% ethanol and then rinsed with sterile ddH₂O.

### 1.4.3 Inoculation Method-1: The Modified “Tube” Method

The cob with the forceps handle is placed in a large Petri plate. A dissecting scope may be used. The top portion (⅔'s) of kernels are cut off and removed with a #10 scalpel (for safety consideration, the cut on the kernels is made by cutting away from your hand that holds the handle of the forceps). The immature embryos are then excised from the kernels on the cob with a scalpel (#11 scalpel): the scalpel blade is inserted on an angle into one end of the kernel. The endosperm is lifted upwards; the embryo is lying underneath the endosperm. The excised embryos are collected in a microfuge tube (or a small Petri plate) containing roughly 1.5 to 1.8 mL of Agrobacterium suspension in LS-inf liquid medium containing acetosyrigone (see above; A-2). Each tube can contain up to 100 embryos. The tube containing embryos is hand-mixed several times, and let the tube/plate stand at room temperature (20 to 25° C.) for 30 min. Remove excess bacterial suspension from the tube/plate with a pipette. Transfer the immature embryos and bacteria in the residue LS-inf medium to a Petri plate containing co-cultivation agar medium. Transfer any immature embryos that remain in the microfuge tube by a sterile loop. Remove excess bacterial suspension with a pipette. A small amount of liquid must be left in the plate to avoid drying out the embryos while plating. Place the immature embryos on the co-cultivation medium with the flat side down (scutellum upward). Do not embed the embryos into medium. Leave the plate cover open in the sterile hood for about 15 min for evaporating excess moisture covering immature embryos. Seal the Petri dishes with 3 M micropore tape. About 100 embryos can be placed on a Petri plate for co-cultivation. Seal the plate and wrap with a sheet of aluminum foil. Incubate the plates in the dark at 22° C. for 2 to 3 days. Take 3 to 5 immature embryos for GUS staining if a GUS construct is used to assess transient GUS expression.

### 1.4.4 Method-2: The “Drop” Method

Excised immature embryos are directly put on the co-cultivation medium (Appendix A-3) with the flat side down (scutellum upward). Each plate (20×100 mm plate) can hold up to 100 immature embryos. Put 5 μL of diluted Agrobacterium cell suspension to each immature embryo with a repeat pipettor. Remove excess moisture covering immature embryos by leaving the plate cover open in the hood for about 15 min. Seal the plate with 3 M micropore tape and wrap with aluminum foil. Incubate the plate in the dark at 22° C. for 2 to 3 days. Take 3-5 immature embryos for GUS staining if a GUS construct is used to assess transient GUS expression.

### 1.4.5 Recovery

After co-cultivation, transfer the embryos to recovery media (A-4) and incubate the plates in dark at 27° C. for about 5 to 10 days. Keep scutellum side up and do not embed into the media.

### 1.4.6 Selection

Transfer immature embryos to 1^{st} selection media (A-6). Roughly 25 to 50 immature embryos can be placed on each plate. Be careful to maintain the same orientation of the embryos (scutellum up). Do not embed the embryos in the media. Seal the Petri plates with white tape. Incubate in the dark at 27° C. for 10 to 14 days (First selection). Subculture all immature embryos that produce variable calli to 2nd selection media (A-6). Try to avoid transferring slimy or soft calli. At this stage, use scissors to remove any shoots that have formed (try to remove the entire embryo from the scutellum if possible and discard it). Firmly place the callus on the media—do not embed into the media. Wrap the plates in 3M Micropore tape and put in the dark at 27° C. Incubate for 2 weeks under the same conditions for the first selection (Second selection). Using 2 pairs of fine forceps, excise the regenerable calli from the scutellum under a stereoscopic microscope. The regenerable calli is whitish/yellowish in color, compact, not slimy and may have some embryo-like structures. Transfer calli to fresh the 2nd selection media (A-6), wrap in 3M Micropore tape and incubate in the dark at 27° C. for 2 weeks. Firmly place the callus on the media—do not embed into the media. Be careful to group and mark the calli pieces that came from the same embryo.

### 1.4.7 Regeneration and Transplanting of Transformed Plants

Excise the proliferated calli (whitish with embryonic structures forming), in the same manner as for 2^{nd} selection and transfer to regeneration media (A-7) in 25×100 mm plates. Firmly place the callus on the media—do not embed into the media. Wrap the plates in 3M Micropore tape and put in the light at 25 or 27° C. Be careful to group the calli pieces that came from the same embryo and number them by embryo.

Incubate under light (ca. 2,000 lux; 14/10 hr light/dark) at 25 or 27° C. for 2 to 3 weeks, or until shoot-like structures are visible. Transfer to fresh regeneration media if necessary. Transfer calli sections with regenerated shoots or shoot-like structures to a Phytatray or Magenta boxes containing rooting medium (A-8) and incubate for 2 weeks under the same condition for the above step, or until rooted plantlets have developed. After 2 to 4 weeks on rooting media, transfer calli that still have green regions (but which have not regenerated seedlings) to fresh rooting Phytatrays. Seedling samples are taken for TaqMan analysis to determine the T-DNA insertion numbers.

Transfer rooted seedlings to Metromix soil in greenhouse and cover each with plastic dome for at least 1 week, until seedlings have established. Maintain the plants with daily watering, and supplementing liquid fertilizer twice a week. When plants reach the 3 to 4 leaf-stages, they are fertilized with Osmocote. If needed putative transgenic plants containing ahas gene are sprayed with 70 to 100 g/ha Pursuit™ by a licensed person, and grown in the greenhouse for another two weeks. Non-transgenic plants should develop herbicidal symptoms or die in this time. Survived plants are transplanted into 10″ pots with MetroMix and 1 teaspoon Osmocote™.

At the flowering stage, the tassels of transgenic plants are bagged with brown paper bags to prevent pollen escape, and the ear shoots are also covered with the ear bag for preventing pollen contamination. Pollination is performed on the transgenic plants. It is best to do self-pollination on the transgenic plants. If silking and anthesis are not synchronized, a wild-type pollen donor or recipient plant with same genetic background as the transgenic T₀ plant should be available for performing cross-pollination. T₁ seeds are harvested, dried and stored properly with adequate label on the seed bag. After harvesting the transgenic T₁ seeds, T₀ plants including the soil and pot should be bagged in autoclave bags and autoclaved (double bagging).

### 1.5 Identification of Single Copy Transgenic Lines (T0) Showing High Expression of Transgene

### 1.5.1 Identification of Single Copy Lines

Single copy lines were identified using TaqMan copy assays (Applied Biosystems Catalog #4326270).

1.5.2 Identification of High Expressing Lines for Transgenes at the mRNA Levels

### 1.5.2.1 Sampling

The nucleic acid samples that were used to determine copy number (see Example 1.5.1, above) were used to assay for transgene (pCER040b & pCER041:I-SceI, pJB034:GU-US, and JB039:GUS) mRNA expression levels.

The T0 leaf nucleic acid samples used for copy number analysis were DNase treated using the DNA-free kit from Ambion (catalog #1906), as described by the manufacturer.

### 1.5.2.2 Expression Analysis Reaction Set-Up

Once samples have been treated with DNase, expression analysis was performed. For analysis of each sample, two reactions were run, one for the gene of interest (either the NOS terminator, GUS reporter gene or I-SceI gene) and one for an endogenous gene control used to quantify RNA concentration in the reaction. The expression of the endogenous gene should remain constant throughout assay conditions so as to accurately reflect relative concentration of the gene of interest. For these experiments a maize gene was identified that shows stable expression levels under normal greenhouse growth conditions (BPS-NC clone ID 62054718). Primers 12 and 13 were used to analyze expression levels of this gene.

| | |
|---|---|
| SEQ ID NO: 13: | Primer 12 (Forward primer Endo): |

5′-TCTGCCTTGCCCTTGCTT-3′

| | |
|---|---|
| SEQ ID NO: 14: | Primer 13 (Reverse primer Endo): |

5′-CAATTGCTTGGCAGGTCTTATTT-3′

The NOS terminator primers anneal before the transcriptional stop in the terminator. The sequences of the primers are below.

| SEQ ID NO: 15: | Primer 14 (Forward primer NOS): |
|---|---|
| 5′-TCCCCGATCGTTCAAACATT-3′ | |
| SEQ ID NO: 16: | Primer 15 (Reverse primer NOS): |

5′-CCATCTCATAAATAACGTCATGCAT-3′

The GUS reporter gene primers anneal in the middle of the gene sequence. The sequences of the primers are below.

| SEQ ID NO: 17: | Primer 16 (Forward primer GUS): |
|---|---|
| 5′-TTACGTGGCAAAGGATTCGAT-3′ | |
| SEQ ID NO: 18: | Primer 17 (Reverse primer GUS): |

5′-GCCCCAATCCAGTCCATTAA-3′

The I-SceI gene primers anneal within the open reading frame. The sequences of the primers are below.

| | |
|---|---|
| SEQ ID NO: 19: | Primer 18 (Forward primer I-SceI): |

5′-GACCAGGTATGTCTGCTGTACGA-3′

| | |
|---|---|
| SEQ ID NO: 20: | Primer 19 (Reverse primer I-Scel): |

5′-CAGGTGGTTAACACGTTCTTTTTT-3′

The reactions were run in a 96-well optical plate (Applied Biosystems, 431-4320), with endogenous control and gene of interest reactions run on the same plate simultaneously. Semi-quantitative RT-PCR using SYBR Green (Eurogentec #RTSNRT032X-1) was performed on the samples using standard procedures known in the art. Reactions were performed on the Perkin Elmer GeneAmp 5700 (serial # 100001042), as described by the manufacturer.

The thermocycler parameters used were as follows:

### Stage 1: 30 min at 48° C. (Reps:1)

### Stage 2: 10 min at 95° C. (Reps:1)

### Stage 3: 15 sec at 95° C. and 1 min at 60° C. (Reps:40)

The default dissociation protocol was used:

### 15 sec at 95° C.

### 20 sec at 60° C.

20 min, 35° C. slow ramp (60-95° C.)

### 1.5.2.3 Data Analysis Results on the GeneAmp5700 for Transgene

The results of the endogenous control reactions were used to confirm the quality and integrity of mRNA samples. Transgene expression was categorized as high, medium, or low in the T0 generation Maize plants based on Ct values generated by GeneAmp5700. High level were regarded as Ct values in the range of 18 to 23, medium levels (Ct values: 24 to 26), low levels (Ct values: 26 to 30). Samples that produced Ct values above 30 were considered to show no transgene expression. Table 9 shows a summary of the number of transgenic lines for each construct, grouped by experimentally determined mRNA expression levels.

TABLE 9
Number of T0 plants in each category of expression
level for each transgene construct.

| Construct | High | Medium | Low | Total tested |
|---|---|---|---|---|
| pJB034 | 36 | 28 | 19 | 83 |
| pJB039 | 56 | 50 | 18 | 124 |
| pJBcer040b | 20 | 2 | 2 | 24 |
| pJBcer041 | 20 | 38 | 29 | 87 |

### 1.6 Proof of Concept on Double Strand Break (DSB)-Mediated Homologous Recombination Via Semi-Transient Assay System

### 1.6.1 Transient Expression Assay for Proof of Concept on DSB-Mediated Recombination

A transient expression assay was used to provide proof of concept data for the DSB-mediated homologous recombination system in plant cells. A GU-US reporter construct (e.g. pJB034) was introduced to maize leaf tissue or protoplasts via biolistic bombardment or PEG-mediated transformation, respectively. The functional GUS open reading frame can only be generated from pJB034 upon homologous recombination of the GU-US locus. The results from these experiments are summarized in Table 10 and in FIG. 4. GUS staining was detected at significantly higher levels when leaf tissue from I-SceI-expressing maize plants was bombarded with pJB034 as compared with maize leaves that did not express I-SceI.

TABLE 10
GUS staining results from transient bombardment assays

| | pJB035 (GUS) | pJB034 (GU-US) |
|---|---|---|
| WT maize | +++ | − |
| CER040b transgenic maize | +++ | ++ |
| CER041 transgenic maize | +++ | + |

### 1.6.2 Biolistic Transformation

The plasmid constructs are isolated using Qiagen plasmid kit (cat# 12143). DNA is precipitated onto 0.6 μM gold particles (Bio-Rad cat# 165-2262) according to the protocol described by Sanford et al. (1993) and accelerated onto target tissues (e.g. two week old maize leaves, BMS cultured cells, etc.) using a PDS-1000/He system device (Bio-Rad). All DNA precipitation and bombardment steps are performed under sterile conditions at room temperature.

Two mg of gold particles (2 mg/3 shots) are resuspended in 100% ethanol followed by centrifugation in a Beckman Microfuge 18 Centrifuge at 2,000 rpm in an Eppendorf tube. The pellet is rinsed once in sterile distilled water, centrifuged, and resuspended in 25 μL of 1 μg/μL total DNA. The following reagents are added to the tube: 220 μL H₂O, 250 μL 2.5M CaCl₂, 50 μL 0.1M spermidine, freebase. The DNA solution is briefly vortexed and placed on ice for 5 min followed by centrifugation at 500 rpm for 5 min in a Beckman Microfuge 18 Centrifuge. The supernatant is removed. The pellet is resuspended in 600 μL ethanol followed by centrifugation for 1 min at 14,000 rpm. The final pellet is resuspended in 36 μL of ethanol and used immediately or stored on ice for up to 4 hr prior to bombardment. For bombardment, two-week-old maize leaves are cut in approximately 1 cm in length and located on 2 inches diameter sterilized Whatman filter paper. In the case of BMS cultured cells, 5 mL of one-week-old suspension cells are slowly vacuum filtered onto the 2 inches diameter filter paper placed on a filter unit to remove excess liquid. The filter papers holding the plant materials are placed on osmotic induction media (N6 1-100-25, 0.2 M mannitol, 0.2 M sorbitol) at 27° C. in darkness for 2-3 hours prior to bombardment. A few minutes prior to shooting, filters are removed from the medium and placed onto sterile opened Petri dishes to allow the calli surface to partially dry. To keep the position of plant materials, a sterilized wire mesh screen is laid on top of the sample. Each plate is shot with 104 of gold-DNA solution once at 2,200 psi for the leaf materials and twice at 1,100 psi for the BMS cultured cells. Following bombardment, the filters holding the samples are transferred onto MS basal media and incubated for 2 days in darkness at 27° C. prior to transient assays. Determine transient expression levels of the reporter gene following the protocols in the art as described above.

### 1.6.3 Protoplast Transfection

Isolation of protoplasts is conducted by following the protocol developed by Sheen (1990). Maize seedlings are kept in the dark at 25° C. for 10 days and illuminated for 20 hours before protoplast preparation. The middle part of the leaves are cut to 0.5 mm strips (about 6 cm in length) and incubated in an enzyme solution containing 1% (w/v) cellulose RS, 0.1% (w/v) macerozyme R10 (both from Yakult Honsha, Nishinomiya, Japan), 0.6 M mannitol, 10 mM Mes (pH 5.7), 1 mM CaCl₂, 1 mM MgCl₂, 10 mM β-mercaptoethanol, and 0.1% BSA (w/v) for 3 hr at 23° C. followed by gentle shaking at 80 rpm for 10 min to release protoplasts.

Protoplasts are collected by centrifugation at 100×g for 2 min, washed once in cold 0.6 M mannitol solution, centrifuged, and resuspended in cold 0.6 M mannitol (2×10⁶/mL).

A total of 50 μg plasmid DNA in a total volume of 100 μL sterile water is added into 0.5 mL of a suspension of maize protoplasts (1×10⁶ cells/mL) and mix gently. 0.5 mL PEG solution (40% PEG 4000, 100 mM CaNO₃, 0.5 mannitol) is added and pre-warmed at 70° C. with gentle shaking followed by addition of 4.5 mL MM solution (0.6 M mannitol, 15 mM MgCl₂, and 0.1% MES). This mixture is incubated for 15 minutes at room temperature. The protoplasts are washed twice by pelleting at 600 rpm for 5 min and resuspending in 1.0 mL of MMB solution [0.6 M mannitol, 4 mM MES (pH 5.7), and brome mosaic virus (BMV) salts (optional)] and incubated in the dark at 25° C. for 48 hr. After the final wash step, collect the protoplasts in 3 mL MMB medium, and incubate in the dark at 25° C. for 48 hr. Determine transient expression levels of the reporter gene following the protocols in the art.

### 1.7 Recovery Marker-Free Transgenic Plants Through Direct Conversion, and Through Callus Culture of F1 Hybrid Immature Embryos

After crossing a transgenic plant containing selection marker bordered by excision sites, and a second transgenic plant containing I-SceI gene, each of the F1 progenies (embryos/seeds) may have: (1) all of the cells in an embryo with intact GOI and selection marker (cell with intact selection marker); (2) all of the cells with GOI, but without selection marker (full marker excision occurred); and (3) some of the cells have the selection marker excised resulting in a mixed genotype.

Depending on the stage excision occurs during and after pollination, there are possibly different genotypes in an embryo/seed. If selection marker excision occurs right at the single cell stage of pollination, a fully excised plant is expected. However, if marker excision event occurs at a later—multi-cell stage during embryo development, one zygote/embryo may contain mixed cell types—cells with and without selection marker.

Conventionally, mature seeds of F1 progeny are harvested, and planted in soil to obtain individual seedlings for future screening. Molecular techniques, such as PCR analysis, and Southern blot analysis are applied to identify the full excision plant.

### 1.7.1 Screening Based on the Conversion of F1 Immature Embryos

In order to save time as compared to the conventional approach through screening mature seeds, a process of converting F1 immature embryos on the rooting medium (A-8) containing the selection agent against the selection marker linked to I-Sce-I gene is applied. For instance, D-serine is applied in the rooting medium if dsdA selection marker is used for generating I-Sce-I plant. Immature embryos are dissected and placed onto the rooting medium (A-8), and then incubated at 27° C. chamber with 16 hr photoperiod. Seedlings recovered are then subjected to molecular screening for identifying the full marker excision plant. For a comparison between the conventional method and the method of immature embryo conversion, about 80 days are saved with the application of immature embryos conversion assuming both methods have the same rate of obtaining full excision events (Table 11).

### 1.7.2 Screening Based on the Callus Culture of F1 Immature Embryos

For increasing the chance of obtaining the full excision event, a process of culturing F1 immature embryos is applied in this invention.

As compared with the approaches of screening F1 plants derived from the mature seeds and from immature embryos, we hypothesize that cell division associated with embryogenic callus culture of immature embryos promotes the activities of marker excision due to the active DNA multiplication and repair activities in the embryogenic callus initiation process. Since regeneration through corn embryogenic callus culture is single cell-based process, a regenerated seedling, therefore, contains a single cell type—e.g. either full excised genotype or non-excised genotype. For example, if the excision occurs only in the scutellum in the F1 immature embryo/seed, it is impossible to recover the full excision plant through screening plants derived from mature seeds or converted immature embryos. In contrast, callus culture of chimeric immature embryo may result in recovering the full excision plant.

TABLE 11
Comparison of time required for conventional and tissue culture regeneration
approaches in obtaining a full marker excision plant.

| | | | | | Recover a full | |
|---|---|---|---|---|---|---|
| | | | Regenerating plants | | marker excision | |
| | Conventional | Time | through callus | Time | plant through | Time |
| | method (through | required | culture from F1 | required | immature embryo | required |
| | mature seeds) | (days) | immature embryos | (days) | conversion | (days) |
| Step 1 | Producing F1 | 70 | Producing F1 | 70 | Producing F1 | 70 |
| | progeny between | | progeny between | | progeny between | |
| | excision target and | | excision target and | | excision target and | |
| | I-SceI parents by | | I-SceI parents by | | I-SceI parents by | |
| | crossing pollination | | crossing pollination | | crossing | |
| | | | | | pollination | |
| Step 2 | Obtaining mature | 50 | Obtaining immature | 10 | Obtaining | 14 |
| | F1 seeds | | embryos (1.0-1.8 mm) | | immature embryos | |
| | | | | | (2-4 mm) | |
| Step 3 | Germinating seeds | 14 | Regenerating plants | 60 | Generating plants | 14 |
| | to obtain F1 | | from scutulum of | | from immature | |
| | seedlings | | immature embryo | | embryos via | |
| | | | through tissue | | embryo conversion | |
| | | | culture | | | |
| Step 4 | Selecting marker- | 7 | Identifying marker- | 7 | Selecting marker | 7 |
| | free plants | | free plants | | free plants | |
| Step 5 | Making F2 progeny | 120 | | | Making F2 | 70 |
| | | | | | immature embryos | |
| Step 6 | Germinating F2 | 14 | | | Generating F2 | 14 |
| | seeds to obtain F2 | | | | plants from | |
| | seedlings | | | | immature embryos | |
| | | | | | via embryo | |
| | | | | | conversion | |
| Step 7 | Identifying marker- | 7 | | | Identifying | 7 |
| | free plants | | | | marker-free plants | |
| | Total | 277 | | 147 | | 196 |

F1 immature embryos of 1 to 1.8 mm in length are dissected onto the recovery medium (A-4, IM medium), and incubated at 27° C. in dark for about 5 to 10 days, and then the derived calli are transferred to and cultured on the selection medium containing the selection agent against the second selection marker gene linked with Sce-I gene for about 14 days. The calli are further cultured on the selection medium for another 14 days, and then transferred to the regeneration medium (A-7) and incubated in a tissue culture chamber under 16 hr/day photoperiod for about 7 to 14 days. The regenerated plants are then transferred to the rooting medium (A-8) under the same condition as the regeneration step. The seedlings are then subjected to molecular screening for identifying the full marker-excision plant.

### 1.7.3 Plant Analysis for DSB-Mediated Homologous Recombination or Marker Excision

### 1.7.3.1 GUS Histochemical Assay

One method that was used to monitor recombination events was histochemical GUS staining in leaf and kernel tissues from pJB034-containing plants. The pJB034 construct comprises an interrupted β-glucuronidase (GUS) reporter gene containing an internal partial sequence duplication such that the functional open reading frame can only be reconstituted via homologous recombination between the repeated sequence. The expression of functional GUS protein in plant tissues can be visualized by means of a chromogenic substrate such as 5-bromo-4-chloro-3-indolyl-β-D-glucuronic acid using methods known to those in the art.

Plant tissues from [JB034 x I-SceI] and [JB034 x I-SceI NULL] plants were analyzed for GUS expression via histochemical staining The results are summarized in Table 12 and FIG. 11. Tissues from plants generated via JB034 x I-SceI crosses showed significantly more GUS expression in leaf and kernels than tissues from plants generated from JB034 x NULL crosses. Crosses with null plants (i.e. plants that do not express I-SceI) showed no detectable GUS staining.

For all combinations, reciprocal crosses were performed with regard to the maternal or paternal transmission of the I-SceI and JB034 constructs; no quantitative or qualitative differences in GUS expression were detectable between maternally or paternally supplied transgenes. Larger amounts of GUS staining were generally observed in plants derived from crosses with CER040b (pUbi::I-SceI) as compared with CER041 (ScBV::I-SceI) plants.

TABLE 12
Results of GUS staining of tissues from JB034 X I-SceI plants

| JB034 crossed with: | GUS Staining | Comments |
|---|---|---|
| JB034 (self) | − | No staining in any tissues |
| CER040b (Ubi::I-SceI) | +++ | Strong staining in leaves |
| | | and kernels (endosperm |
| | | and scutellum) |
| CER041 (ScBV::I-SceI) | ++ | Strong staining in leaves |
| | | and less intense staining in |
| | | kernels (endosperm) |
| Null | − | No staining in any tissues |

Leaf tissue from different JB034 x I-SceI plants varied in the amount of GUS staining that was visualized: from spotty GUS staining representing recombination events that happened relatively late in the leaf development, streaks of GUS staining representing tissue developed from cells that had previously undergone recombination, to fully blue leaves representing recombination events that occurred at a developmental stage preceding leaf formation. Since recombination occurs at the cellular level, it was possible that different leaves from the same plant would yield different GUS staining patterns.

### 1.7.3.2 PCR Analysis for Marker Excision

PCR was used to provide molecular characterization of recombination events from plants comprising either pJB034 or pJB039 reporter constructs.

Plants from JB034 crosses were analyzed by PCR using primers 7 and 8, which are based in the ScBV promoter and a region of the GUS ORF that is downstream of the repeated region, respectively.

| SEQ ID NO: 21: | Primer 7 (ScBV fwd): |
|---|---|
| 5′-GATCGCAGTGCGTGTGTGACACC-3′ | |
| SEQ ID NO: 22: | Primer 8 (GUS AS879 rev): |

5′-GTCCGCATCTTCATGACGACC-3′

In order to maximize assay throughput, genomic DNAs were grouped into pools for PCR analyses. PCR amplification with primers 7 and 8 from the native JB034 construct yields a 1.7 kb product, while amplification from recombined JB034 generates a 1.0 Kb PCR product. FIG. 12 shows that PCR performed with genomic DNA from [JB034 x I-SceI] plants yielded both the 1.7 kb and 1.0 kb products; when the template was genomic DNA from selfed JB034 results in generation of only the 1.7 kb product expected from the unrecombined JB034 locus. Individual plants from the genomic DNA pools that yielded the excision-specific PCR product were subsequently analyzed separately.

Plants from JB039 crosses were analyzed by PCR using primers 9 and 10, which are based in the AHAS open reading frame and the region adjacent to the T-DNA Right border, respectively. PCR with primers 9 and 10 should result in a 6.7 kb product from a native JB039 template, and a 0.9 kb product from the proposed recombined JB039. PCR was carried out under conditions that would not allow efficient amplification of the 6.7 kb native product, so in order to confirm that the genomic DNA was intact for all samples, an additional PCR was performed in order to provide an easily amplifiable product from genomic DNA comprising the native JB039 construct. This confirmatory PCR used primers 9 and 11 generates a 1.2 kb product from native JB039, and no product from recombined JB039, due to the loss of the primer 11 homologous sequence.

| | |
|---|---|
| SEQ ID NO: 23: | Primer 9 (AHAS ORF fwd): |

5′-CTAATGGTGGGGCTTTCAAGG

| | |
|---|---|
| SEQ ID NO: 24: | Primer 10 (RB proximal rev): |

5′-CCTTAAGGCGATCGCGCTGAGGC

| | |
|---|---|
| SEQ ID NO: 25: | Primer 11 (distal AHAS term rev): |

5′-AGTGTACGGAATAAAAGTCC

In order to efficiently analyze genomic DNA from as many plants as possible, the plant genomic DNAs from [JB039 x I-SceI] or [JB039 x null] plants were pooled and initially assayed as such. FIG. 13 shows typical results of these PCR analyses. Individual plants from the genomic DNA pools that yielded the excision-specific PCR product were subsequently analyzed separately.

### 1.7.3.3 Plant Analysis for Marker Excision

A series of 17 crosses were generated for the GU-US reporter construct (JB034). A total of 369 events from 8 crosses were obtained with the Ubiquitin I-SceI construct (CER40b: Zm.ubiquitin promoter::Zm.ubiquitin intron::I-SceI::NOS terminator) and 520 events from 9 crosses obtained with the ScBV I-SceI construct (CER41: ScBV promoter::I-SceI::NOS terminator). Histochemical screening of the Ubiquitin I-SceI cross produced 118 positive recombination events indicated by blue streaks and spots, an average of 15 recombined events per cross. PCR analysis of 354 of these events produced 58 positive PCR products, an average of 7 recombined events per cross. When the crosses were made using the ScBV I-SceI 14 histochemically positive events out of 520 were obtained, an average of 2 recombined events per cross. The PCR analysis identified 28 positive PCR products out of 516 events, an average of 4 recombined events per cross (Table 3). The weaker ScBV promoter resulted in fewer recombination events born out by both histochemical and PCR screening methods. A JB034 crossed to an I-SceI null line yielded 93 events of which 1 was histochemically and PCR positive. The positive event is possibly a spontaneous recombination event or the result of an error.

A similar series of crosses were made for the JB039 lines and the two I-SceI constructions. The 4 Pseudo GUS x Ubiquitin I-SceI crosses yielded 188 events of which 18 produced a positive PCR product, an average of 5 positive events per cross. The other 7 crosses using the ScBV-I-SceI construct yielded 434 events of which only 8 were PCR positive, an average of 1 recombined event per cross (Table 13). A subset of the events was histochemically stained however no white streak or spots could be distinguished in the intense blue background so further staining was abandoned. As with the interrupted GUS construct the use of the weaker ScBV promoter resulted in approximate 4 fold lower recombination events.

TABLE 13
Molecular screening results from all regenerated plants.

| GU-USxUbq-SceI | | | Pseudo GUSxUbq-SceI | |
|---|---|---|---|---|
| 8 Crosses | Stained | PCR | 4 Crosses | PCR |
| Total Events | 369 | 354 | Total Events | 118 |
| Positive¹ | 118 | 58 | Positive | 18 |
| Average² | 15 | 7 | Average | 5 |
| GU-USxScBV-SceI | | | Pseudo GUSxScBV-SceI | |
| 9 Crosses | Stained | PCR | 7 Crosses | |
| Total Events | 520 | 516 | Total Events | 434 |
| Positive | 14 | 28 | Positive | 8 |
| Average | 2 | 4 | Average | 1 |
| GU-USxI-SceI null | | | Pseudo GUSx I-SecI null | |
| 1 Cross | Stained | PCR | 4 Crosses | |
| Total Events | 93 | 93 | Total Events | 172 |
| Positive | 1 | 1 | Positive | 0 |

Positive¹ recombination was indicated by either blue spots or streaks and positive PCR events were indicated by an appropriate sized band. All results were chimeric in nature due to the pooling of tissue from same event and/or the incomplete excision obtained within any individual plant.
The average² is expressed as events per cross.

A total of 132 out of 834 regeneration events containing the GU-US reporter construct, JB034 stained positive for recombination while 86 out of 870 events produced a PCR band indicative of the recombined gene. Of these positive events 50 were positive for both criteria (Table 14). The one positive event obtained in the I-SceI null cross was either a spontaneous event or an error.

TABLE 14
Summary of events obtained with regeneration and screening results. Summary of
the events obtained from each type of cross analyzed either histochemically or with PCR.
GU-US events screened differed for each test as tissue for every plant for the histochemical
analysis was not always available at the time of event sampling. The histochemical analysis
of the excision events was discontinued and PCR analysis was performed exclusively.

| | Total # Events | | | | |
|---|---|---|---|---|---|
| | GUS Histochemical | # Events | Total # Events | # Events | # Events both |
| Cross | assays tested | Stain | PCR tested | PCR+ | Stained & PCR+ |
| JB034xI-SceI | 834 | 132 | 870 | 86 | 50 |
| JB034xI-SceInull | 93 | 1 | 93 | 1 | 1 |
| JB039xI-SceI | 28 | 0 | 622 | 26 | |
| JB039xI-SceInull | 76 | 0 | 172 | 0 | |

1.7.4 Retransformation Strategy for Producing Plants with Both I-SceI and Excision Target

We also evaluated another approach of obtaining putative marker excision events: re-transforming the excision target plant with a construct containing the I-SceI gene with a second selection marker. Since the tissue culture process may promote the excision activities (hypothesis), re-transformation experiments were conducted with several testing constructs (HEN constructs: JB084, LM319 or LM320).

To perform the re-transformation experiments, we followed the transformation procedure described above using the excision target plant (with AHAS as the selection marker) as the transformation donor material, and applying the selection agent against the selection cassette for the second transformation construct (e.g. D-Ser for dsdA gene).

When we used a strong, constitutive, ubiquitous promoter to express the HEN gene and GU-US, no DSB-mediated HR, (indicated by blue spots) was detected in embryo axis in maize upon analysis of mature kernel. To achieve DSB-mediated HR in embryo, the super promoter was chosen, since this promoter in maize shows strong expression in the whole embryo (scutellum and embryo axis) during germination, calli (including embryogenic calli) during regeneration. The expression levels in these tissues can be enhanced by addition of an intron-mediated enhancement (IME)-conferring intron between the super promoter and I-SceI gene.

### 1.7.4.1 Embryo-Specific Promoter

In order to maximize I-SceI expression in the embryos of developing maize kernels, vectors were generated that comprise the expression cassettes wherein the I-SceI gene is driven by the super promoter, a promoter that has been described to drive high levels of expression in these tissues.

Vector pJB082 is a pUC based vector that comprises a p-Super::I-SceI::t-Nos expression cassette, and was generated by the 3-way ligation of the T4 DNA polymerase filled in HindIII-BglII super promoter fragment from pLM266, the T4 DNA polymerase filled in AscI-SbfI fragment of pJB010, and the T4 DNA polymerase filled in AscI fragment of pCER039. How was JB084 finally assembled?

The binary vector pLM319 comprises the p-Super::I-SceI::t-Nos cassette, and was generated by ligation of the T4 DNA polymerase filled in PacI-PmeI fragment of pJB082 into T4 DNA polymerase filled in AscI digested pLM151.

An expression cassette comprising p-Super::I-Ubi::I-SceI::t-Nos was generated in a pUC vector backbone by ligating in the T4 DNA polymerase filled in BglII-AscI ubiquitin intron fragment from pLM303 into the T4 DNA polymerase filled in SphI digested pJB082, thereby generating pJB083. The binary vector pLM320 was generated by ligation of the T4 DNA polymerase filled in PacI-PmeI p-Super::I-Ubi::I-SceI::t-Nos fragment from pJB083 into T4 DNA polymerase filled in AscI digested pLM151.

### 1.7.4.2 Retransformation of Reporter Events

A homozygous event for each reporter construct, JB034 and JB039 underwent embryo rescue and was re-transformed with the I-SceI gene driven by the Super promoter, JB084. This promoter is believed to give higher expression in the germinating embryo and scutellum layer, which may improve the recovery of recombined plants. A similar retransformation set was performed with RLM319 and RLM320 using embryos from JB034 homozygous events. A total of 112 embryos were transformed with RLM319 and 100 embryos were transformed with RLM320.

### 1.7.4.3 Plant Analysis for Marker Excision

A total of 16 lines containing the pseudo-marker gene, JB039 were recovered from the retransformation experiment with JB084. Nine lines were stained at the five-leaf stage and examined for white patches. Three lines showed leaves with a half white half blue pattern. The rest showed fully blue leaves. Leaves from six lines including one that had previously shown the half white pattern were stained at pollination and all showed fully blue leaves. A total of 11 lines containing the interrupted GUS gene, JB034 were recovered from the re-transformation experiment with JB084. All were stained for recombination but none showed any blue staining.

A total of 20 lines containing the interrupted GUS gene, JB034 were recovered from two retransformation experiments with LM319. A total of 28 lines containing the interrupted GUS gene, JB034 were recovered from two retransformation experiments with LM320. These retransformed plants were screened for the presence of the selectable marker and GUS stained to screen for recombination. Nine LM319 and 15 LM320 plants were GUS stained to screen for recombination. No homologous recombination positive events were recovered from the transformants produced using the Super promoter without the intron, JB084 or RLM319. Eleven positive events, with 3 being completely blue in tissue culture were identified from the transformation using the Super promoter with the intron, RLM320 (Table 15, FIGS. 5 and 6). Re-transformation data generated with the super promoter constructs indicated that super promoter in combination with intron (i.e Maize Ubiquitin intron in LM320) is effective in driving the expression of I-SceI gene to a functional level. Without this intron (LM319), the super promoter is ineffective.

TABLE 15
The construct used for each transformation and the number of
confirmed lines. The GUS staining showed recombination occurred
with the Super promoter coupled with the Ubiquitin intron while
no recombinants were obtained using the super promoter without
the intron.

| First | | | Number of | Recombined | Fully |
|---|---|---|---|---|---|
| Con- | Second | embryos | confirmed | events/# of | recombined |
| struct | Construct | infected | events | events | events |
| JB039 | JB084 | | 12 | 3/9 | 0 |
| JB034 | JB084 | | 11 | 0/11 | 0 |
| JB034 | LM319 | 112 | 7 | 0/9 | 0 |
| JB034 | LM320 | 100 | 12 | 11/15 | 3/15 |

JB034 transgenic plants were re-transformed with RLM320 or JB084 followed by selfing to set seed. None of the progeny containing JB084 showed homologous recombination. Leaves from a total of 15 T0 events containing RLM320 were tested for homologous recombination. Eleven out of 15 events showed homologous recombination via both GUS histochemical assay and PCR. Three out of 11 were fully recombined. Five out of the 11 events were tested in T1 generation. Three to four plants per event were analysed. Two out of a total of 12 T1 plants were fully recombined.

### 2. Application of Minimaize as an Efficient Tool for Determining Frequency of Marker Excision in Maize

In order to reduce the time to obtain the marker-free transgenic plants, a rapid cycling dwarf maize line can be utilized. This transformable dwarf line offers advantages over regular maize lines because it's small size and short life cycle—it completes a life cycle from seed to seed in about 60 days as compared to 120 days for the regular maize lines. This line is extremely useful in determining the HEN's marker excision efficiency in maize.

Transformation experiments are conducted mainly based the protocol with agrobacterium-mediated transformation procedure described in the Example 3. On the other hand, transformation experiments can also be conducted based on direct DNA delivery methods such as a biolistic transformation, e.g. particle bombardment known to the skilled in the art. Preparation of transformation donor materials also follows the procedure described above.

### BRIEF DESCRIPTION OF THE DRAWINGS

FIG. 1. Vector pCER 040b. Binary I-SceI expression vector comprising the maize ubiquitin promoter/intron cassette driving the expression of I-SceI.

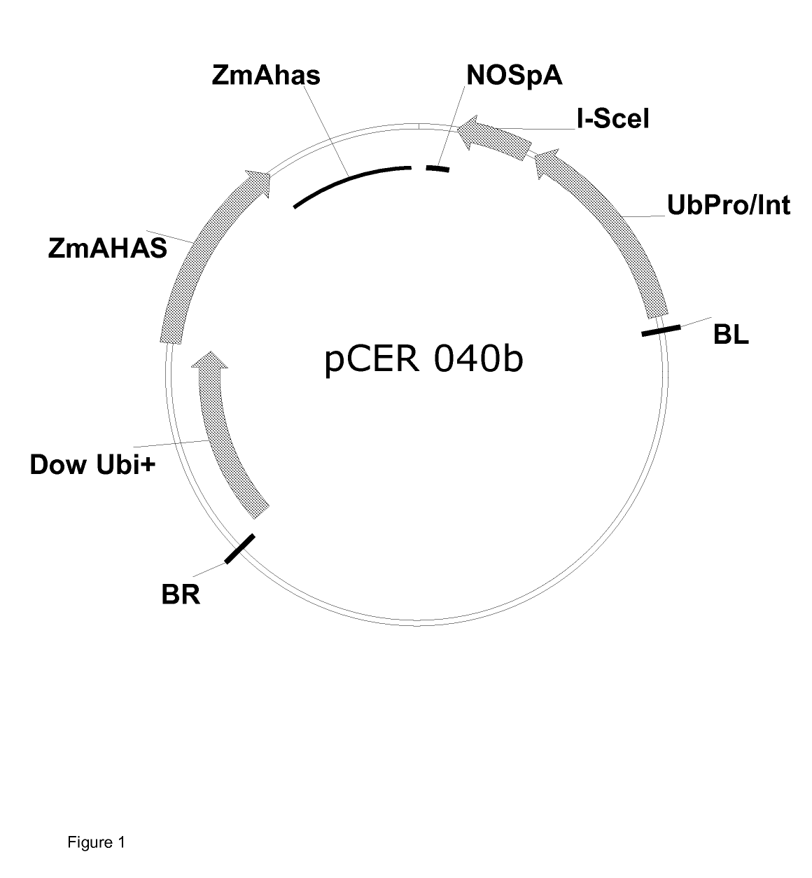

FIG. 2. Vector pCER 041. Binary I-SceI expression vector comprising the ScBV promoter driving the expression of I-SceI

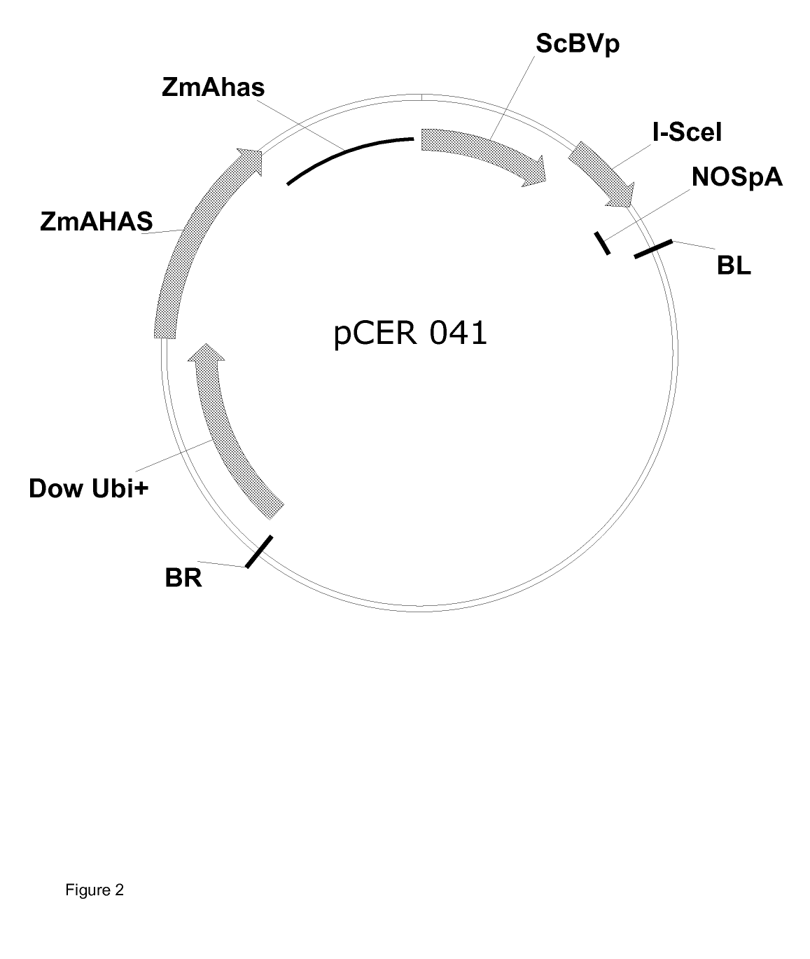

FIG. 3. Vector pJB 034. Binary reporter vector comprising the GU-US reporter cassette.

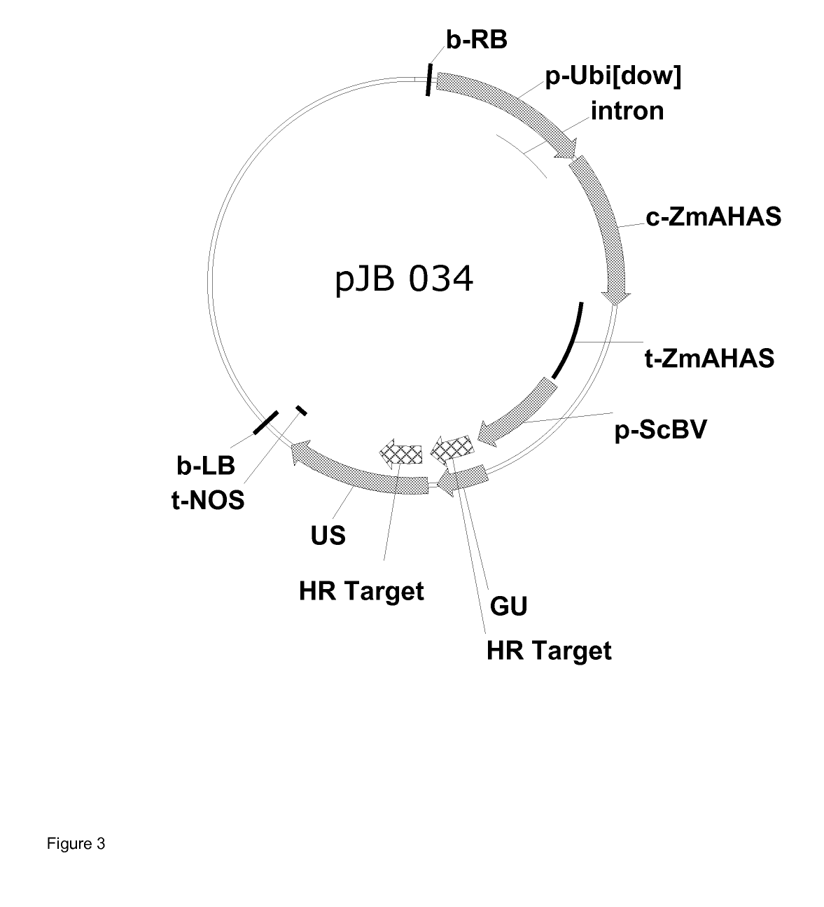

FIG. 4. Vector pJB 039. Binary reporter vector comprising the pseudo-marker excision cassette.

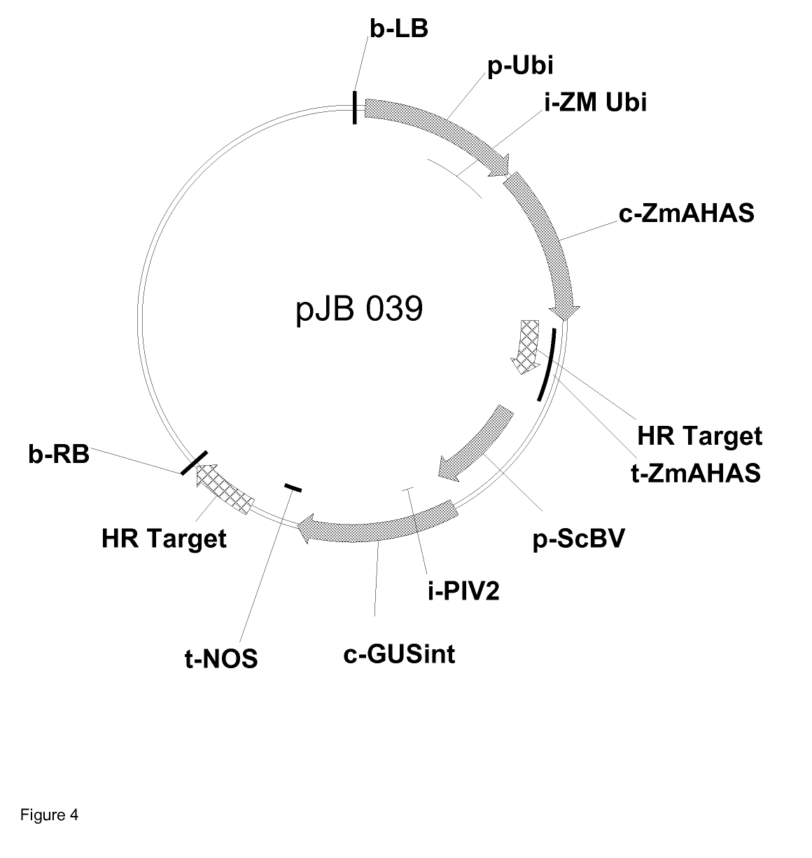

FIG. 5. Vector pLM 319. Binary I-SceI expression vector comprising the Super promoter driving the expression of I-SceI.

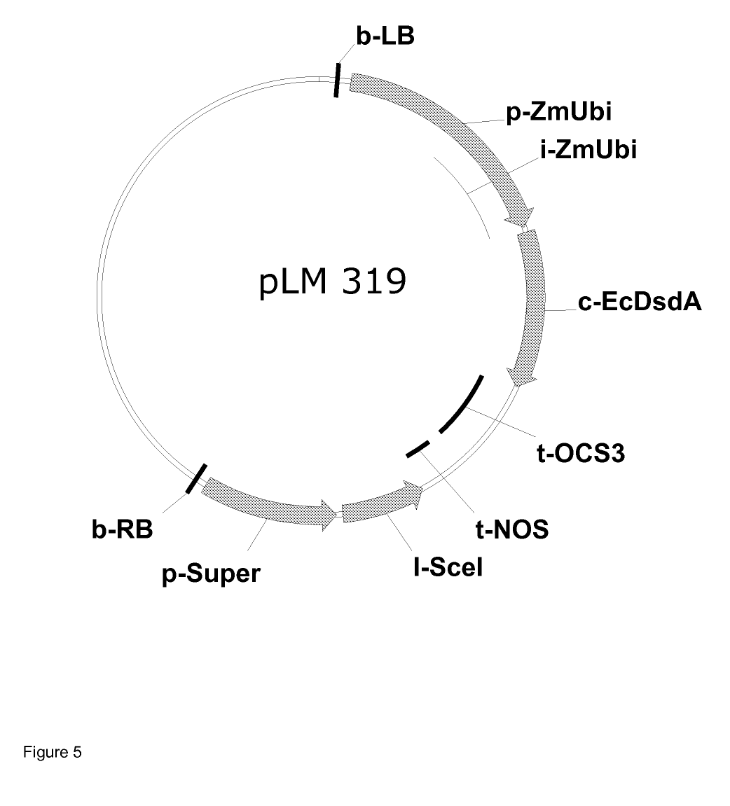

FIG. 6. Vector pLM 320. Binary I-SceI expression vector comprising I-SceI expression driven by the Super promoter in conjunction with the Zm ubiquitin intron.

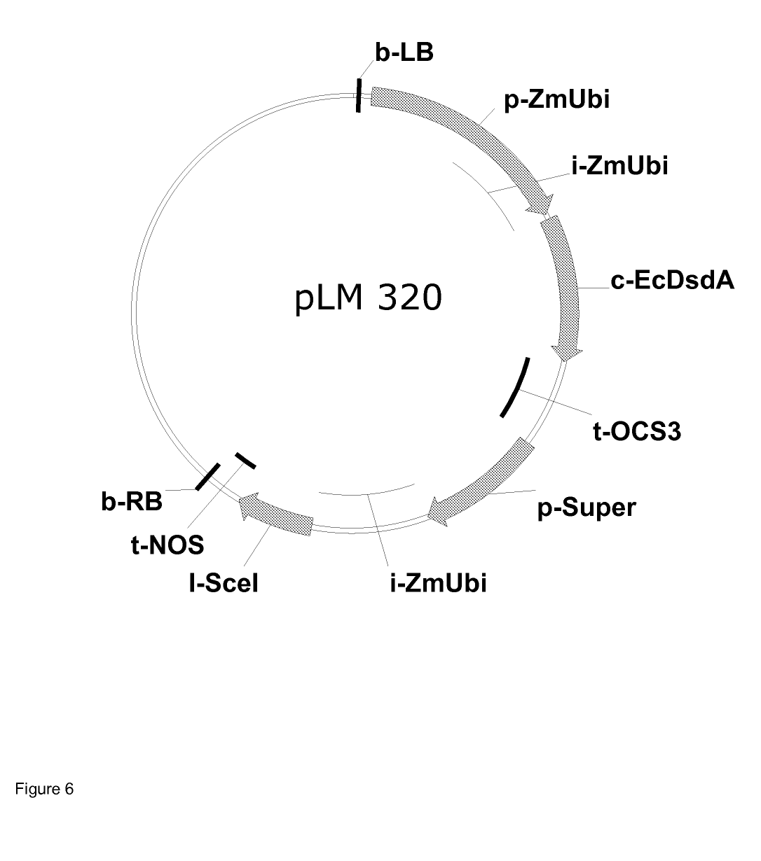

FIG. 7. A diagram of the constructs used for DSB-induced homologous recombination. (A) GU-US construct encodes an expression cassette wherein the GUS ORF comprises an internal duplication (i.e. 650 by of GUS coding sequence: hatched bars), with an I-SceI recognition site located between the duplicated regions (GU-US). (B) Pseudo-marker excision vector comprises a duplicated DNA sequence (i.e. 850 by of AHAS terminator region of the selectable marker cassette: gray bars) flanking the I-SceI sites in order to serve as a target sequence for homologous recombination (HR target). (C) I-SceI construct comprises the I-SceI expression cassette. The T-DNA regions for all of these vectors also comprises a selectable marker cassette (SMS) in addition to the above described elements.

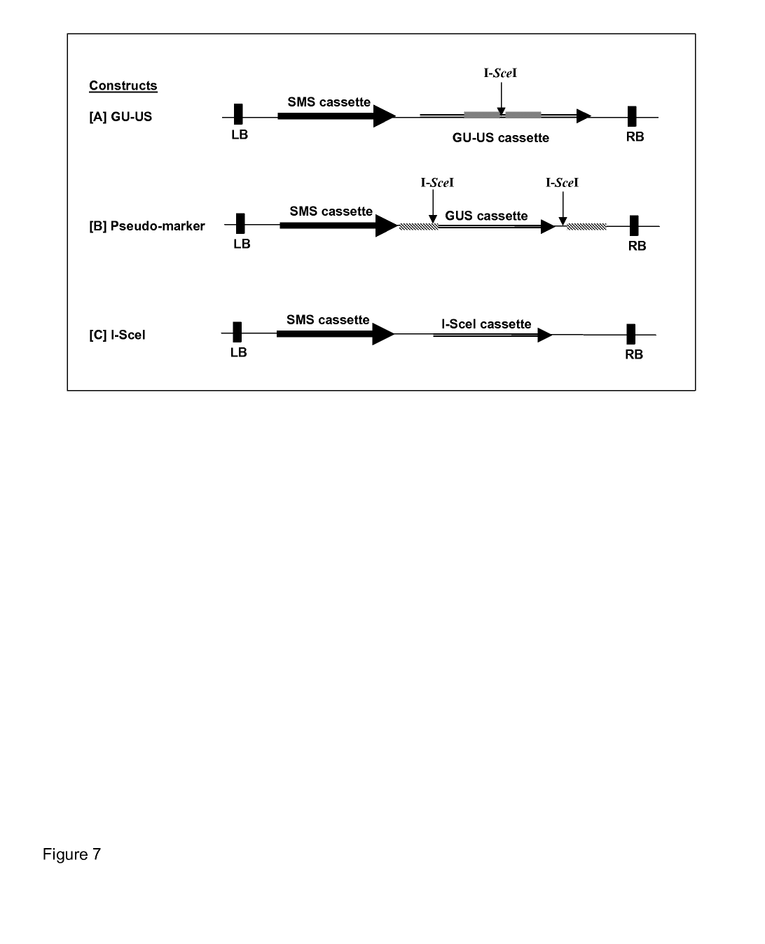

FIG. 8. A selection process of identifying T0 lines that showing potential DSB-mediated HR using transient assays. Medium to high expressing lines comprising I-SceI (or GU-US) were transferred with GU-US (or I-SceI) construct. The lines showing GUS histochemical positive expression (blue spots) were selected. Young embryos in T0 plants were used for immature embryo conversion to identify homozygous lines, which sped up at least 1.5 months compared to the conventional maize breeding timeline, because the seed development and maturation, seed-drying time is omitted. This transient assay process including immature embryo conversion allows not only a reduction in the overall time requirement but also an increased frequency of identification of candidate lines that show the potential for exhibiting a high rate of homologous recombination.

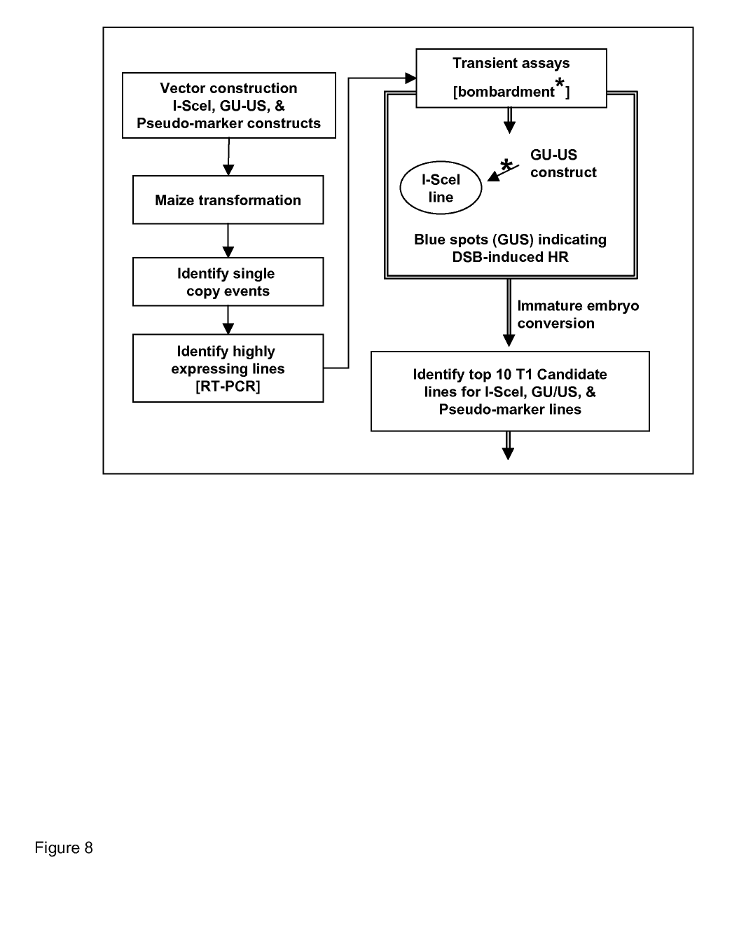

FIG. 9. Approaches for identifying DSB-induced HR occurring in transgenic maize lines. Each method requires various range of time to obtain transgenic lines exhibiting DSB-mediated HR: Conventional method using crossing (A) requires minimum 19 months. Regeneration (B) and retransformation (C) methods require approximately 8-9 months. ***implies the transient assay system described in FIGS. 8 and 10.

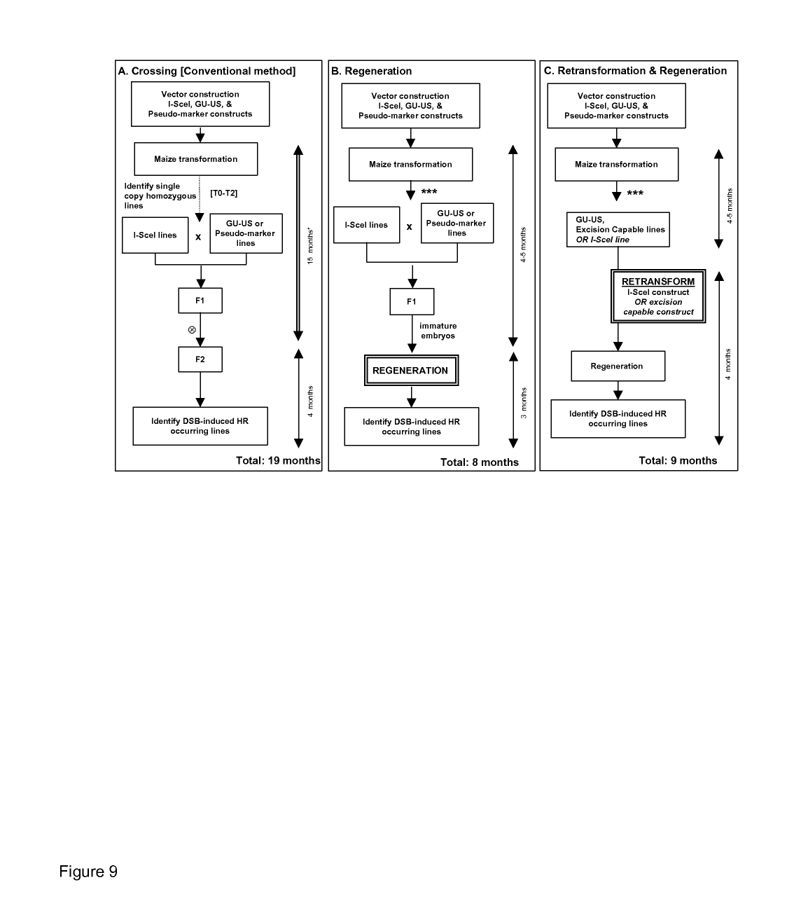

FIG. 10. Transient assay for DSB-induced homologous recombination in maize leaf tissue. (A) Wild type maize leaf tissue was bombarded with vectors comprising expression cassettes for either a functional GUS ORF (left) or the GU-US ORF (right), confirming that expression of GU-US does not result in detection of functional GUS by histochemical staining. (B) Bombardment of leaf tissues from I-SceI expressing plants with the GU-US expression cassette results in the generation of detectable GUS by histochemical staining This result was seen in maize plants using both the ubiquitin promoter (left) and the ScBV promoter (right) to drive I-SceI expression.

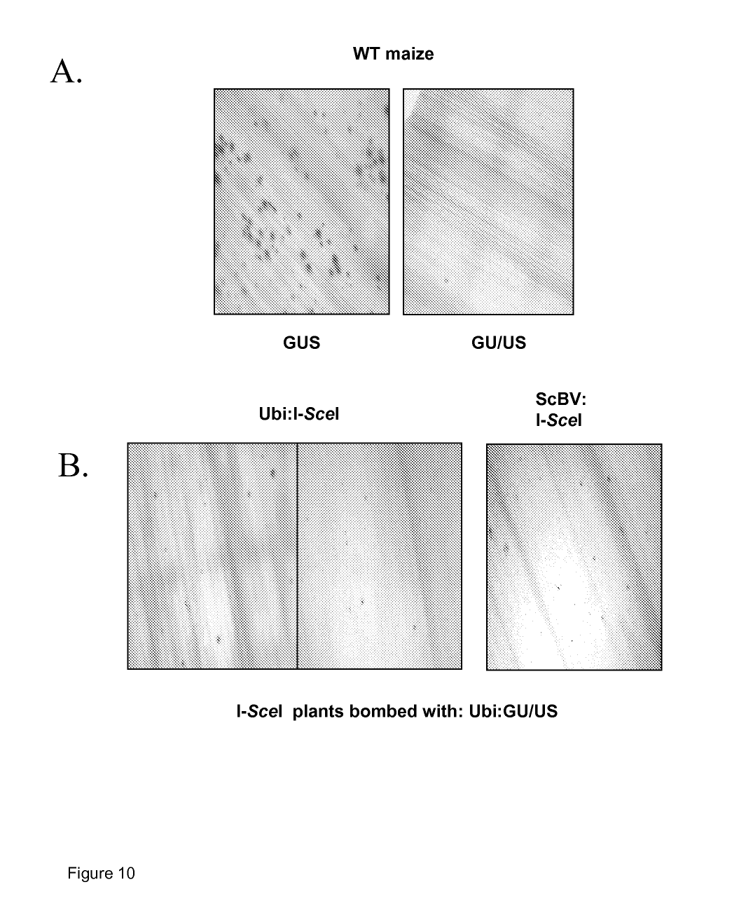

FIG. 11. Histochemical analysis of [JB034 X I-SceI] plant kernels. (A) Histochemical staining of kernels generated by crossing maize lines harboring the GU-US expression cassette with maize lines expressing I-SceI shows the generation of functional GUS. There is no GUS staining when seeds are analyzed from homozygous I-SceI expressing plants (bottom right well in left plate). (B) Histochemical staining of homozygous GU-US kernels demonstrates that in the absence of I-SceI expression, there is no generation of functional GUS protein.

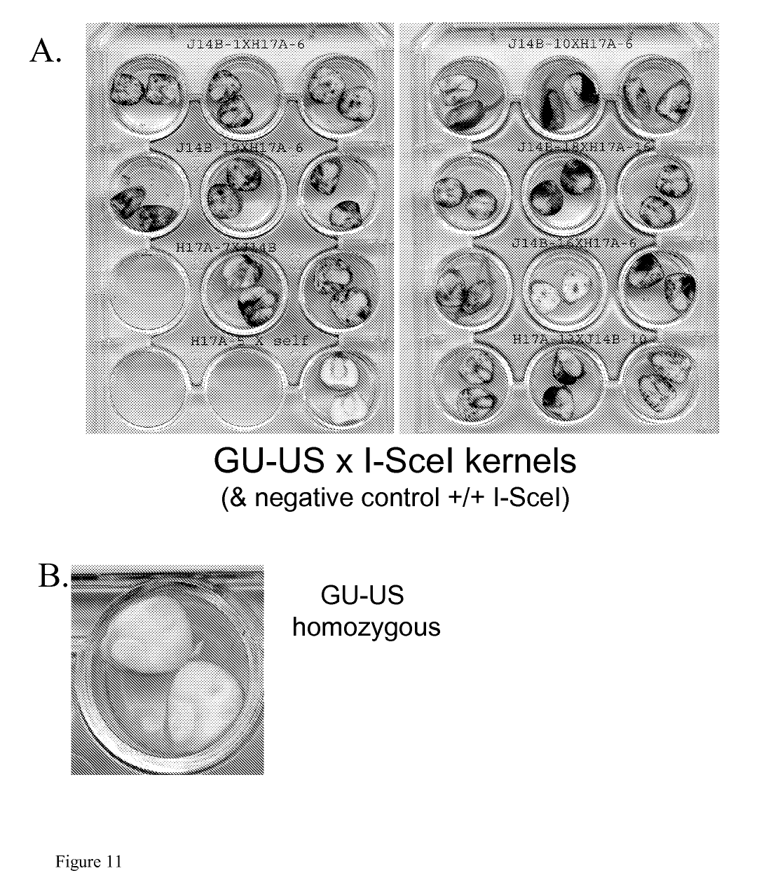

FIG. 12. Genomic PCR of [JB034 X I-SceI] plants, pooled samples. (A) Genomic DNA samples were prepared from [GU-US X I-SceI] plants and pooled. Similar pools of genomic DNA were prepared from self pollinated GU-US plants. Genomic PCR was performed as described in the examples. Positive control reactions using purified pJB034 vector generated the 1.7 Kb product expected from the native construct; this reaction also generated a 1.0 Kb product indicative of the recombined locus, indicating a low level of vector recombination during bacterial passages. Vector pCER044 is equivalent to vector pJB034 following homologous recombination at the GU-US locus, and yields the expected 1.0 Kb PCR product. PCR amplification with genomic DNA from wild type maize plants does not result in the generation of any PCR product, demonstrating that the PCR products generated in these reactions are specific for the JB034 locus. Analysis of the genomic DNA pools shows that the 1.7 Kb unrecombined product is produced with both the [JB034 X I-SceI] and the homozygous JB034 pools, but the recombined 1.0 Kb product is only produced when the [JB034 X I-SceI] genomic DNA is used as a template. (B) Histochemical staining of kernel and leaf samples from homozygous JB034 plants (left) and [JB034 X I-SceI] plants (right), showing that homologous recombination of the GUS locus only is detectable in plants that express I-SceI.

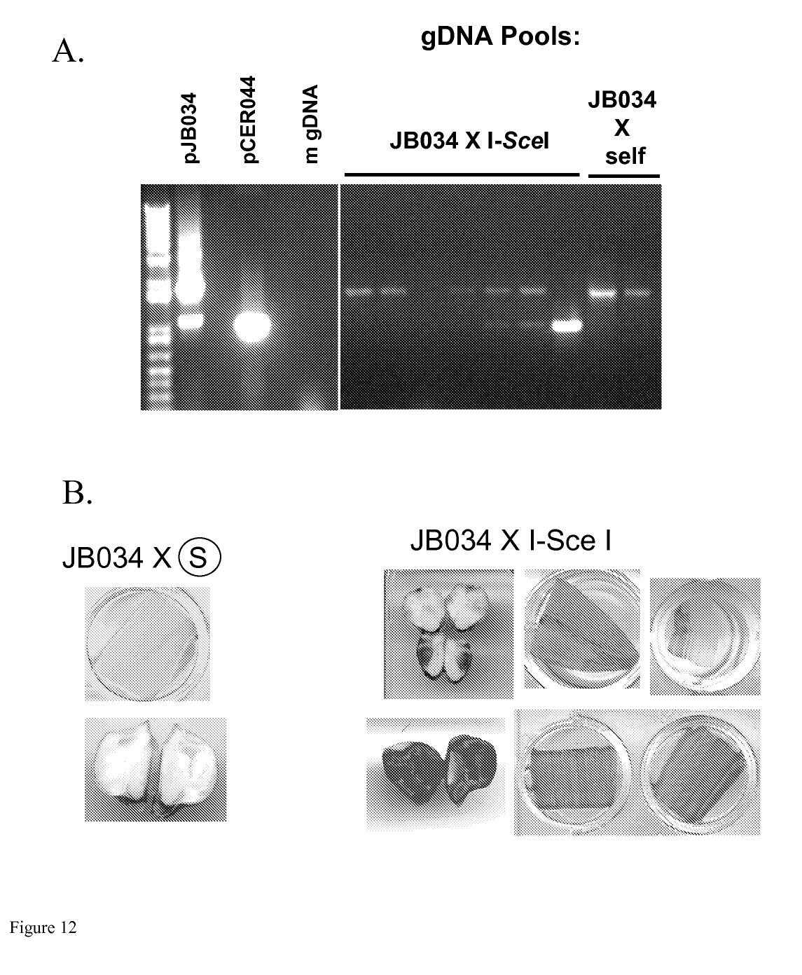

FIG. 13. Genomic PCR of [JB039 X I-SceI] plants, pooled samples. Genomic DNA pools were generated for [JB034 X I-SceI] (blue) and [JB034 C null] (red) plants. PCR was performed using primers 9 and 10 to confirm the presence of the JB039 template and with primers 9 and 11 to detect plants comprising cells that have undergone homologous recombination at this locus (left). PCR with primers 9 and 10 generates the expected 1.2 Kb product from all samples, indicating that all genomic DNA pools comprise the native JB039 locus (top right). PCR with primers 9 and 11 generate the 0.9 Kb product only in genomic DNA pools A2 and A4, each assembled from the [JB039 X I-SceI] crosses (bottom right).

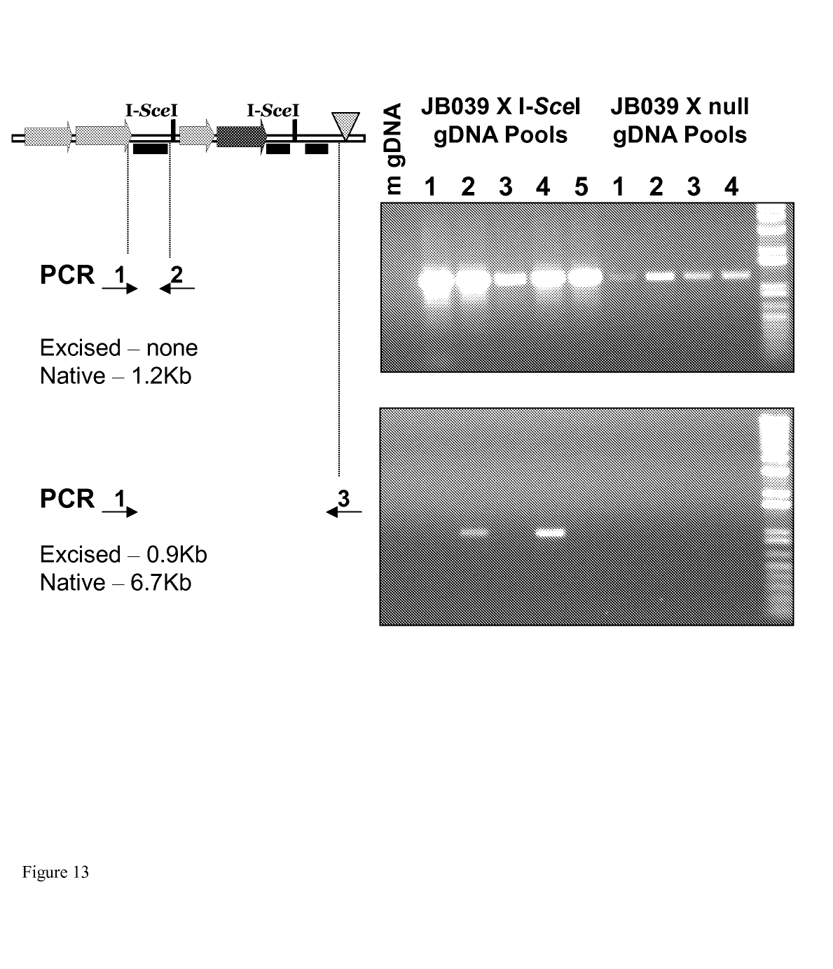

FIG. 14. Genomic PCR of individual [JB039 x I-SceI] plants. Genomic DNA from individual [JB039 x I-SceI] plants was analyzed by PCR using primer combinations 9:10 (top) and 9:11 (bottom) as described in the examples. The control reactions (no DNA, vector control pJB039, and wild type maize genomic DNA) all produced the expected products from both primer sets. Analysis of genomic DNA pool A2 identified two individual plants that comprise the recombined JB039 locus, ie: plants 8a and 9b. Analysis of the plants that make up genomic DNA pool A4 shows that only plant 17b comprises the recombined locus. Lanes labeled 15a and 109 represent genomic DNA samples from individual [JB039 X I-SceI] plants that were not included in the previous genomic DNA pools.

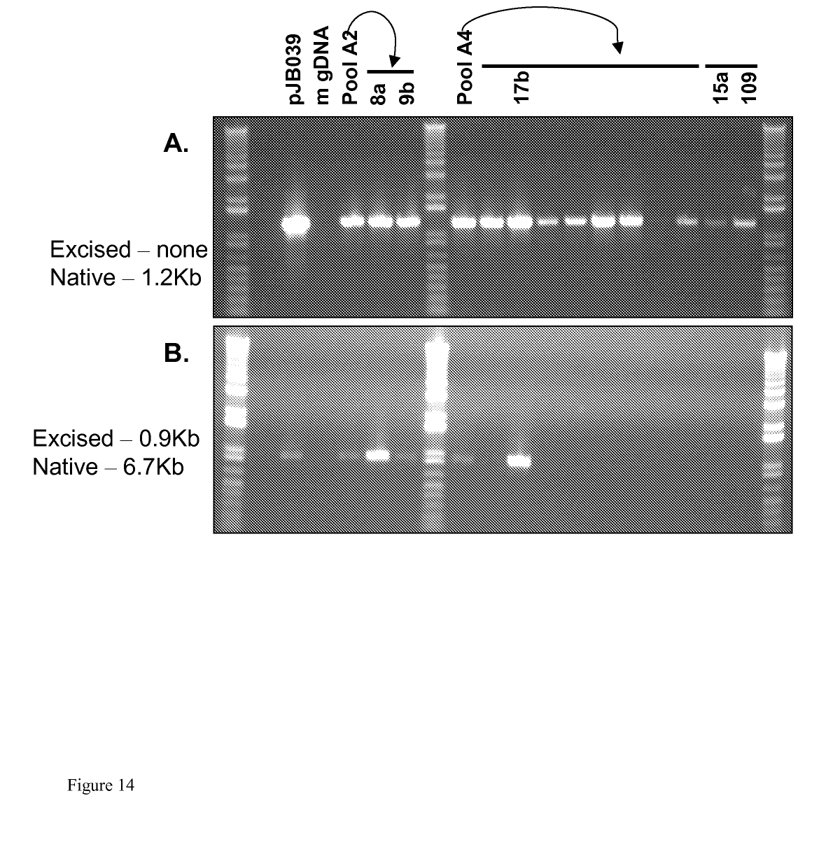

FIG. 15. Graphic representation of the synthetic homing endonuclease I-SceI gene sequences. The Gateway attachment regions, Att-L1 and Att-L2 are depicted by the hashed boxes. The Kozak consensus is indicated by the vertical arrow and the open reading frame by the solid arrow. The location of selected restriction sites is indicated.

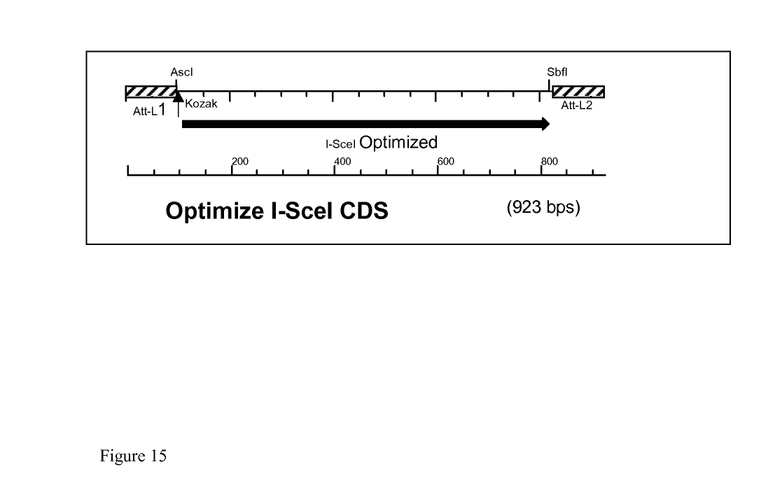

1. A method for excising a nucleic acid sequence from the genome of a plant or of a plant cell, comprising:
a) transforming a plant cell with a construct encoding a DNA double strand break inducing enzyme,
b) generating a transgenic plant line from the cell of step a),
c) performing a transient assay with the plant line of step b) or cells or parts thereof to analyze the functionality of the transgenic DNA double strand break inducing enzyme,
d) crossing the plant line of step b) with a plant line containing a nucleic acid sequence to be excised, wherein the nucleic acid sequence to be excised comprises at least one recognition sequence which is specific for the enzyme of step a) for the site-directed induction of DNA double strand breaks, and wherein the nucleic acid sequence to be excised is bordered at both sides by a repeated sequence which allows for a DNA repair mechanism, and
e) performing either an immature embryo conversion or a tissue culture regeneration through callus formation.

2. A method for excising a nucleic acid sequence from the genome of a plant or of a plant cell, comprising:
a) transforming a plant cell with a construct encoding a nucleic acid sequence to be excised, wherein the nucleic acid sequence to be excised comprises at least one recognition sequence which is specific for a DNA double strand break inducing enzyme for the site-directed induction of DNA double strand breaks, and wherein the nucleic acid sequence to be excised is bordered at both sides by a repeated sequence which allows for a DNA repair mechanism,
b) generating a transgenic plant line from the cell of step a),
c) performing a transient assay with the plant line of step b) or cells or parts thereof to analyze the functionality of the recognition sequence and the repeated sequence of the construct of step a),
d) crossing the plant line of step b) with a plant line containing a DNA double strand break inducing enzyme, and
e) performing either an immature embryo conversion or a tissue culture regeneration through callus formation.

3. The method according to claim 1, wherein the DNA repair mechanism is homologous recombination.

4. The method according to claim 1, further comprising the identification of a single copy transgenic line following step b) or c).

5. The method according to claim 4, wherein the identification of a single copy transgenic line is performed via molecular techniques including quantitative PCR or Southern hybridization.

6. The method according to claim 1, further comprising the analysis of the transgene expression level following step b) or c).

7. The method according to claim 6, wherein the analysis of the transgene expression level is performed via molecular techniques including RT-PCR or Northern hybridization.

8. The method according to claim 1, further comprising the pollination of the transgenic plant line following step b) or c), wherein the pollination is either self-pollination or cross-pollination with a wild-type line.

9. The method according to claim 8, wherein seeds and/or seedlings obtained through the pollination are analyzed for their zygosity.

10. The method according to claim 9, wherein homozygous lines identified after the zygosity analysis are selected for the crossing of step d).

11. The method according to claim 1, wherein the transformation of step a) is selected from the group consisting of Agrobacterium mediated transformation, biolistic transformation, protoplast transformation, polyethylene glycol transformation, electroporation, sonication, microinjection, macroinjection, vacuum filtration, infection, and incubation of dried embryos in DNA-containing solution.

12. The method according to claim 1, wherein the transient assay of step c) is an intrachromosomal homologous recombination assay.

13. The method according to claim 1, wherein the transient assay of step c) comprises a transient transformation of a reporter construct.

14. The method according to claim 13, wherein the reporter construct is selected from the group consisting of a GUS construct, a green fluorescent protein construct, a chloramphenicol transferase construct, a luciferase construct, a beta-galactosidase construct, an R-locus gene product construct, a beta-lactamase construct, a xyl E gene product construct, an alpha amylase construct, a tyrosinase construct and an aequorin construct.

15. The method according to claim 13, wherein the reporter construct comprises a nucleic acid sequence to be excised, wherein the nucleic acid sequence comprises at least one recognition sequence which is specific for the enzyme of step a) for the site-directed induction of DNA double strand breaks, and wherein the nucleic acid sequence is bordered at both ends by a repeated sequence which allows for DNA repair mechanisms including homologous recombination.

16. The method according to claim 1, wherein seeds and/or seedlings obtained by step e) are analyzed for DNA double strand break mediated repair mechanisms including homologous recombination.

17. The method according to claim 16, wherein the analysis of DNA repair mechanisms including homologous recombination in the seeds and/or seedlings is determined by molecular techniques including PCR analyses, colorimetric or biochemical assays, or DNA sequencing.

18. The method according to claim 1, wherein the DNA double strand break inducing enzyme is selected from the group consisting of homing endonucleases, restriction endonucleases, group II endonucleases, recombinases, transposases and chimeric endonucleases.

19. The method according to claim 1, wherein the DNA double strand break inducing enzyme is selected from the group consisting of I-SceI, F-SceI, F-SceII, F-SuvI, F-TevI, F-TevII, I-AmaI, I-AniI, I-CeuI, I-CeuAIIP, I-ChuI, I-CmoeI, I-CpaI, I-CpaII, I-CreI, I-CrepsbIP, I-CrepsbIIP, CrepsbIIIP, I-CrepsbIVP, I-CsmI, I-CvuI, I-CvuAIP, I-DdiI, I-DdiII, I-DirI, I-DmoI, HmuI, I-HspNIP, I-LlaI, T-MsoI, I-NaaI, T-NanI, I-NclIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PpbIP, I-PpoI, I-SPBetaIP, I-Seal, I-SceII, I-SceIII, I-SceIV, I-SceV, I-SceVI, I-SceVII, I-SexIP, I-SneIP, I-SpomCP, I-SpomIP, I-SpomIIP, I-SquIP, I-Ssp68031, I-SthPhiJP, I-SthPhiST3P, T-SthPhiS3bP, 1-TevI, RTI I-TevII, I-TevIII, I-UarAP, I-UarHGPAIP, I-UarHGPA13P, I-ZbiIP, PI-MtuI, PI-MtuHIP, PT-MtuHIIP, PI-PfuI, PI-PfuII, PI-PkoI, PI-PkoII, PT-PspI, PT-Rma43812IP, PI-SPBetaIP, PI-SceI, PT-TfuI, PI-TfuII, PI-ThyI, PT-TliI and PI-TliII.

20. The method according to claim 1, wherein the DNA double strand break inducing enzyme is selected from the group consisting of enzymes having an amino acid sequence as depicted in SEQ ID NOs: 26 or 27 or a substantial homologue thereof.

21. The method according to claim 1, wherein the construct of step a) is selected from the group consisting of a vector, a plasmid, a cosmid, a bacterial construct or a viral construct.

22. The method according to claim 21, wherein the vector is selected from the group consisting of pCB series, pLM series, pJB series, pCER series, pEG series, pBR series, pUC series, M13mp series and pACYC series.

23. The method according to claim 1, wherein the construct of step a) comprises a promoter for the expression of the DNA double strand break inducing enzyme.

24. The method according to claim 23, wherein the promoter is selected from the group consisting of constitutive promoters, development-dependent promoters, plant virus derived promoters, inducible promoters, chemically inducible promoters, biotic or abiotic stress inducible promoters, pathogen inducible promoters, tissue specific promoters, promoters with specificity for the embryo, scutellum, endosperm, embryo axis, anthers, ovaries, pollen, meristem, flowers, leaves, stems, roots, seeds, fruits and/or tubers, promoters which enable seed specific expression in monocotyledons including maize, barley, wheat, rye and rice, super promoters, and functional combinations of such promoters.

25. The method according to claim 23, wherein the promoter is selected from the group consisting of a ubiquitin promoter, sugarcane bacilliform virus promoter, phaseolin promoter, 35S CaMV promoter, 19S CaMV promoter, short or long USB promoter, Rubisco small subunit promoter, legumin B promoter, nopaline synthase promoter, TR dual promoter, octopine synthase promoter, vacuolar ATPase subunit promoter, proline-rich protein promoter, PRP1 promoter, benzenesulfonamide-inducible promoter, tetracycline-inducible promoter, abscisic acid-inducible promoter, salicylic acid-inducible promoter, ethanol inducible promoter, cyclohexanone inducible promoter, heat-inducible hsp80 promoter, chill-inducible alpha-amylase promoter, wound-induced pinII promoter, 2S albumin promoter, legumin promoter, unknown seed protein promoter, napin promoter, sucrose binding protein promoter, legumin B4 promoter, oleosin promoter, Bce4 promoter, high-molecular-weight glutenin promoter, gliadin promoter, branching enzyme promoter, ADP-glucose pyrophosphatase promoter, synthase promoter, bgp1 promoter, lpt2 or lpt1 promoter, hordein promoter, glutelin promoter, oryzin promoter, prolamine promoter, gliadin promoter, glutelin promoter, zein promoter, kasirin promoter, secalin promoter, ory s1 promoter, ZM13 promoter, Bp10 promoter, Lcg1 promoter, AtDMC1 promoter, class I patatin promoter, B33 promoter, cathepsin D inhibitor promoter, starch synthase promoter, GBSS1 promoter, sporamin promoter, tomato fruit-specific promoter, cytosolic FBPase promoter, ST-LSI promoter, CP12 promoter, CcoMT1 promoter, HRGP promoter, super promoter, promoters in combination with an intron-mediated enhancement conferring intron, and functional combinations of such promoters.

26. The method according to claim 23, wherein the promoter comprises a nucleic acid sequence as depicted in nucleotides 1 to 1112 of SEQ ID NO: 6.

27. The method according to claim 1, wherein the nucleic acid to be excised comprises the sequence of the T-DNA region or part thereof or encodes a selection marker or part thereof.

28. The method according to claim 27, wherein the selection marker is selected from the group consisting of negative selection markers, markers conferring resistance to a biocidal metabolic inhibitor, to an antibiotic or to a herbicide, positive selection markers and counter-selection markers.

29. The method according to claim 27, wherein the selection marker is selected from the group consisting of acetohydroxy acid synthase, D-serine deaminase, phosphinothricin acetyltransferase, 5-enolpyruvylshikimate-3-phosphate synthase, glyphosates degrading enzymes, dalapono inactivating dehalogenases, sulfonylurea- and imidazolinone-inactivating acetolactate synthases, bromoxynilo degrading nitrilases, Kanamycin- or G418-resistance genes, neomycin phosphotransferase, 2-desoxyglucose-6-phosphate phosphatase, hygromycin phosphotransferase, dihydrofolate reductase, D-amino acid metabolizing enzyme, D-amino acid oxidase, gentamycin acetyl transferase, streptomycin phosphotransferase, aminoglycoside-3-adenyl transferase, bleomycin resistance determinant, isopentenyltransferase, beta-glucoronidase, mannose-6-phosphate isomerase, UDP-galactose-4-epimerase, cytosine deaminase, cytochrome P-450 enzymes, indoleacetic acid hydrolase, haloalkane dehalogenase and thymidine kinase.

30. The method according to claim 1, wherein the plant is selected from the group consisting of maize, Arabidopsis, sorghum, rice, rapeseed, tobacco, wheat, rye, barley, oat, potato, tomato, sugar beet, pea, sugarcane, asparagus, soy, alfalfa, peanut, sunflower and pumpkin.

31. The method according to claim 1, wherein the crossing of step d) is replaced by a re-transforming of the plant line of step b) with a construct encoding a nucleic acid sequence to be excised, wherein the nucleic acid sequence to be excised comprises at least one recognition sequence which is specific for the enzyme of step a) for the site-directed induction of DNA double strand breaks, and wherein the nucleic acid sequence to be excised is bordered at both sides by a repeated sequence which allows for a DNA repair mechanism.

32. The method according to claim 2, wherein the crossing of step d) is replaced by a re-transforming of the plant line of step b) with a construct encoding a DNA double strand break inducing enzyme.

33. A plant obtained by the method according to claim 1, or progeny, propagation material, a part, tissue, cell or cell culture derived from said plant.

34. (canceled)

35. Aliment, fodder or seeds comprising the plant or progeny, propagation material, part, tissue, cell or cell culture according to claim 33.

36. A process for the production of pharmaceuticals or chemicals, comprising utilizing the plant or progeny, propagation material, part, tissue, cell or cell culture according to claim 33.


SEQUENCE LISTING (29 sequences)

SEQ ID NO: 1 - DNA, 14869 bp, Artificial sequence
  Composite binary vector LB-Ubiquitin promoter/intron::ZmAHAS cds::ZmAHAS 3′/term-[t-nos::I-SceI cds::Zm ubiquitin promoter/intron] reverse-RB
aaactgaaggcgggaaacgacaatcagatctagtaggaaacagctatgaccatgattacgccaagctgcgatcgccaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcctgcaggttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatggcgcgggggatcctctagagtcgacctgcaggcatgcaagcttggcgcgccttaattaaaggcctgttgggggtaccctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaactattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagccgcggaagcttgcatgcctgcaggcatgcaagcttggcgcgccttaattaaaggcctgttaacagccgcgccactagtcaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttgtggaattcgagctcggtacccggggatcctctagagtcgacctgcaggcatgcaagctttgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacccccggggatccactagttctagaaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatgcccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctaatggtggggctttcaaggatatgatcctggatggtgatggcaggactgtgtactgatctaaaatccagcaagcaactgatctaaaatccagcaagcaccgcctccctgctagtacaagggtgatatgttttatctgtgtgatgttctcctgtgttctatctttttttgtaggccgtcagctatctgttatggtaatcctatgtagcttccgaccttgtaattgtgtaggtctgttgttttccttctggcatgtgtcataagagatcatttaagtgtccttttgctacatataaataagataataagcactgctatcgagtggttctgaattggcttctgttgccaaatttaagtgtccaactggtccttgcttttgttttcgctatttttttttccttttttagttattattatattggtaatttcaactcaacatatgatgtatggaataatactagggctgcaatttcaaactattttacaaactagaatggcattttcgtggtttgaggggggtgaaaaaatgatcaagagtggcatttgactgaattagttacctgatccaaagaaataatccattggngccccacttgcatccctaaggctagtttgaaaacataaatctttaatccctaaagctaatttagaaccattggngccccacttgcatccctaaggctagtttgaaaacataaatctttaatccctaaagctaatttattttcgtggttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattctaatttctctaaaacgaagggatctaaacgccctctaaagctaatttggaaactcaaactttsttagcattggaggggattgagaaaaaatattaattcattttcatctcaatcattcaatctccaaagagatttgagttccttattagtctgttccatgcatcaaatcggctcaatgtgtcattatttgccatgacgattgacgagttgttctggggcctagcgctttccacgccgatgtgctggggcctggtcctggagaagacagcttgatatttaaagctatcaattgtttcaattgattcccacttcatttttctaaatgtagaaaacggtgacgtataagaaaaagaatgaattaggcttttattccgtacactaatctagagcggccccttaagagcgctgcgatcgcgttaacagcttgctgaggaggcctcggaccgtta

SEQ ID NO: 2 - DNA, 14180 bp, Artificial sequence
  Composite binary vector RB-Ubiquitin promoter/intron::ZmAHAS cds::ZmAHAS 3′/term-ScBV promoter::I-SceI::Nos terminator-LB
taatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaaggtaccgggccccccctcgaggtcgacggtatcgataagcttgatcgcgccatgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctagcctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataacctgcaggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgatcgcagcttggcgtaatcatggtcatagctgtttcctactagatctgattgtcgtttcccgccttcagtttcgcgccactagtcaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttgtggaattcgagctcggtacccggggatcctctagagtcgacctgcaggcatgcaagctttgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacccccggggatccactagttctagaaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatgcccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctaatggtggggctttcaaggatatgatcctggatggtgatggcaggactgtgtactgatctaaaatccagcaagcaactgatctaaaatccagcaagcaccgcctccctgctagtacaagggtgatatgttttatctgtgtgatgttctcctgtgttctatctttttttgtaggccgtcagctatctgttatggtaatcctatgtagcttccgaccttgtaattgtgtaggtctgttgttttccttctggcatgtgtcataagagatcatttaagtgtccttttgctacatataaataagataataagcactgctatcgagtggttctgaattggcttctgttgccaaatttaagtgtccaactggtccttgcttttgttttcgctatttttttttccttttttagttattattatattggtaatttcaactcaacatatgatgtatggaataatactagggctgcaatttcaaactattttacaaactagaatggcattttcgtggtttgaggggggtgaaaaaatgatcaagagtggcatttgactgaattagttacctgatccaaagaaataatccattggngccccacttgcatccctaaggctagtttgaaaacataaatctttaatccctaaagctaatttagaaccattggngccccacttgcatccctaaggctagtttgaaaacataaatctttaatccctaaagctaatttattttcgtggttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattctaatttctctaaaacgaagggatctaaacgccctctaaagctaatttggaaactcaaactttsttagcattggaggggattgagaaaaaatattaattcattttcatctcaatcattcaatctccaaagagatttgagttccttattagtctgttccatgcatcaaatcggctcaatgtgtcattatttgccatgacgattgacgagttgttctggggcctagcgctttccacgccgatgtgctggggcctggtcctggagaagacagcttgatatttaaagctatcaattgtttcaattgattcccacttcatttttctaaatgtagaaaacggtgacgtataagaaaaagaatgaattaggcttttattccgtacactaatctagagcggccccttaagagcgctgcgatcgcgttaacagcttgctgaggaggcctcggaccgttaat

SEQ ID NO: 3 - DNA, 15801 bp, Artificial sequence
  Composite binary vector RB-Ubiquitin promoter/intron::ZmAHAS cds::ZmAHAS 3′/term-ScBV promoter::GU fragmentI-SceI siteUS fragment::Nos terminator-LB
gtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttgtggaattcgagctcggtacccggggatcctctagagtcgacctgcaggcatgcaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacccccggggatccactagttctagaaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctaatggtggggctttcaaggatatgatcctggatggtgatggcaggactgtgtactgatctaaaatccagcaagcaactgatctaaaatccagcaagcaccgcctccctgctagtacaagggtgatatgtttttatctgtgtgatgttctcctgtattctatctttttttgtaggccgtcagctatctgttatggtaatcctatgtagcttccgaccttgtaattgtgtagtctgttgttttccttctggcatgtgtcataagagatcatttaagtgccttttgctacatataaataagataataagcactgctatgcagtggttctgaattggcttctgttgccaaatttaagtgtccaactggtccttgcttttgttttcgctatttttttccttttttagttattattatattggtaatttcaactcaacatatgatgtatggaataatgctagggctgcaatttcaaactattttacaaaccagaatggcattttcgtggtttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattctaatttctctaaaacgaagggatctaaacgccctctaaagctaatttggaaactcaaactttcttagcattggaggggattgagaaaaaatattaattcattttcatctcaatcattcaatctccaaagagatttgagttccttattagtctgttccatgcatcaaatcggctcaatgtgtcattatttgccatgacgattgacgagttgttctggggcctagcgctttccacgccgatgtgctggggcctggtcctggagaagacagcttgatatttaaagctatcaattgtttcaattgattcccacttcatttttctaaatgtagaaaacggtgacgtataagaaaaagaatgaattaggacttttattccgtacactaatctagagcggccccttaaggcgctgcgatcgcgttaacagcttgctgaggaggcctcggaccgttaattaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaaggtaccgggccccccctcgaggtcgacggtatcgataagcttgatctagtctagagtcgatcgaccatggtacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtgccagcggccgcctagggataacagggtaatagtctagtccgaaaacgccgtgagacatattggttacgatcctaaggtagcgaaattcacccggtaactctgtgccagctagagtcctgtagaaaccccaacccgtgaaatcaaaaaactcgacggcctgtgggcattcagtctggaccgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaagcggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggacttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaataagcttatcgataccgtcgacctcgagcgccactagtcaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctcc

SEQ ID NO: 4 - DNA, 16447 bp, Artificial sequence
  Composite binary vector LB-Ubiquitin promoter/intron::ZmAHAS cds::ZmAHAS 3′/term-ScBV promoter::GUS cds::Nos terminator-HR repeat-RB
tggcaggatatattgtggtgtaaacaaattgacgcttagacaacttaataacacattgcggacgtttttaatgtactgaattgactagtggcgcgccaagcttgcatgcctgcaggcatgcaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacccccgggggatccactagttctagaaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctaatggtggggctttcaaggatatgatcctggatggtgatggcaggactgtgtactgatctaaaatccagcaagcaactgatctaaaatccagcaagcaccgcctccctgctagtacaagggtgatatgtttttatctgtgtgatgttctcctgtattctatctttttttgtaggccgtcagctatctgttatggtaatcctatgtagcttccgaccttgtaattgtgtagtctgttgttttccttctggcatgtgtcataagagatcatttaagtgccttttgctacatataaataagataataagcactgctatgcagtggttctgaattggcttctgttgccaaatttaagtgtccaactggtccttgcttttgttttcgctatttttttccttttttagttattattatattggtaatttcaactcaacatatgatgtatggaataatgctagggctgcaatttcaaactattttacaaaccagaatggcattttcgtggtttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattctaatttctctaaaacgaagggatctaaacgccctctaaagctaatttggaaactcaaactttcttagcattggaggggattgagaaaaaatattaattcattttcatctcaatcattcaatctccaaagagatttgagttccttattagtctgttccatgcatcaaatcggctcaatgtgtcattatttgccatgacgattgacgagttgttctggggcctagcgctttccacgccgatgtgctggggcctggtcctggagaagacagcttgatatttaaagctatcaattgtttcaattgattcccacttcatttttctaaatgtagaaaacggtgacgtataagaaaaagaatgaattaggacttttattccgtacactaatctagagcggccgccaccgcggtggagctcgaattccacgtgttaattaacggtccgaggcctcctcagcaagctgttaacgcgatcgcagcgccttaaggggcccgtttaacaggcctttaattaccctgttatccctaattaaggcgcgccaagcttgcatgcctgcaggaagttgaagacaaagaaggtcttaaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaagggggtaggtcagtcccttatgttacgtcctgtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttgtgggcgcgccaagcttgcatgcctgcaggtcgatagggataacagggtaatctagaggatccccgggtacctctaaaatccagcaagcaactgatctaaaatccagcaagcaccgcctccctgctagtacaagggtgatatgtttttatctgtgtgatgttctcctgtattctatctttttttgtaggccgtcagctatctgttatggtaatcctatgtagcttccgaccttgtaattgtgtagtctgttgttttccttctggcatgtgtcataagagatcatttaagtgccttttgctacatataaataagataataagcactgctatgcagtggttctgaattggcttctgttgccaaatttaagtgtccaactggtccttgcttttgttttcgctatttttttccttttttagttattattatattggtaatttcaactcaacatatgatgtatggaataatgctagggctgcaatttcaaactattttacaaaccagaatggcattttcgtggtttgaggggagtgaaaaaaaatgaggcatttgactgaattagttacctgatccattttcgtggtttggatcattggaattaaattccattctaataatagtaattttggcatatatcaattaagttaattcggttttatgcaaaatatatttgtatactattattatcaagatgtcggagatatttatatgctacatttttactatacaggagtgagatgaagagtgtcatgtaagttacacagtagaaacaaattctattaatgcataaaatcatttccatcatccaccctatgaatttgagatagacctatatctaaactttgaaaagtggttgaatatcaaattccaaattaaataagttattttattgagtgaattccacgtgcggtccgcctcagcgcgatcgccttaaggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagggggggggggggggggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaacccccccccccccccccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcccccgatcaagtagatacactacatatatctacaatagacatcgagccggaaggtgatgtttactttcctgaaatccccagcaattttaggccagtttttacccaagacttcgcctctaacataaattatagttaccaaatctggcaaaagggttaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggcctcaacatctagtcgcaagctgaggggaaccactagtgtcatacgaacctccaagagacggttacacaaacgggtacattgttgatgtcatgtatgacaatcgcccaagtaagtatccagctgtgttcagaacgtacgtccgaattaattcatcggggtacggtcgacgatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 5 - DNA, 11457 bp, Artificial sequence
  Composite binary vector LB-Ubiquitin promoter/intron::Ec DsdA cds::OCS3 3′/term-[t-nos::I-SceI cds::super promoter] reverse-RB
gtgattttgtgccgagctgccggtcggggagctgttggctggctggtggcaggatatattgtggtgtaaacaaattgacgcttagacaacttaataacacattgcggacgtttttaatgtactgaattggatccgcccgggcggtaccaagcttccgcggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacggatcctcatctaagcgcaaagagacgtactatggaaaacgctaaaatgaactcgctcatcgcccagtatccgttggtaaaggatctggttgctcttaaagaaaccacctggtttaatcctggcacgacctcattggctgaaggtttaccttatgttggcctgaccgaacaggatgttcaggacgcccatgcgcgcttatcccgttttgcaccctatctggcaaaagcatttcctgaaactgctgccactggggggattattgaatcagaactggttgccattccagctatgcaaaaacggctggaaaaagaatatcagcaaccgatcagcgggcaactgttactgaaaaaagatagccatttgcccatttccggctccataaaagcacgcggcgggatttatgaagtcctggcacacgcagaaaaactggctctggaagcggggttgctgacgcttgatgatgactacagcaaactgctttctccggagtttaaacagttctttagccaatacagcattgctgtgggctcaaccggaaatctggggttatcaatcggcattatgagcgcccgcattggctttaaggtgacagttcatatgtctgctgatgcccgggcatggaaaaaagcgaaactgcgcagccatggcgttacggtcgtggaatatgagcaagattatggtgttgccgtcgaggaaggacgtaaagcagcgcagtctgacccgaactgtttctttattgatgacgaaaattcccgcacgttgttccttgggtattccgtcgctggccagcgtcttaaagcgcaatttgcccagcaaggccgtatcgtcgatgctgataaccctctgtttgtctatctgccgtgtggtgttggcggtggtcctggtggcgtcgcattcgggcttaaactggcgtttggcgatcatgttcactgcttttttgccgaaccaacgcactccccttgtatgttgttaggcgtccatacaggattacacgatcagatttctgttcaggatattggtatcgacaaccttaccgcagcggatggccttgcagttggtcgcgcatcaggctttgtcgggcgggcaatggagcgtctgctggatggcttctatacccttagcgatcaaaccatgtatgacatgcttggctggctggcgcaggaagaaggtattcgtcttgaaccttcggcactggcgggtatggccggacctcagcgcgtgtgtgcatcagtaagttaccaacagatgcacggtttcagcgcagaacaactgcgtaataccactcatctggtgtgggcgacgggaggtggaatggtgccggaagaagagatgaatcaatatctggcaaaaggccgttaataacgtttcaacgcagcatggatcgtaccgagctcaatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatgggcccaggtacagaattcgcggccgtacaacgcgtaccggttaattaatctagaggcgcgaaactgaaggcgggaaacgacaatcagatctagtaggaaacagctatgaccatgattacgccaagctgcgatcgccaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcctgcaggttatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggctagccagtttgttgaaagcttggtgtttgaaggtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgtttttcatggcgcgggggatcctctagagtcgacctgcaggcatgcaagcttggcgcggatcttcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctccctcgatcgagaaagagatcaatgttgagctgcttcaaaagcaatgggattgaccagctcgcggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatccgcgagcttatcgataccgtcgaatctagaggatccgcccaaagctgcgccttacgcgccgggcccggccggccagatcttgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccgggggggggggggggggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaccccccccccccccccctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcccccgatcaagtagatacactacatatatctacaatagacatcgagccggaaggtgatgtttactttcctgaaatccccagcaattttaggccagtttttacccaagacttcgcctctaacataaattatagttaccaaatctggcaaaagggttgaccggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcagggatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtg

SEQ ID NO: 6 - DNA, 3164 bp, Artificial sequence
  SuperPromoter::Ubiquitin intron::I-SceI::Nos terminator cassette that replaces the reverse orientation [Super promoter::I-SceI::Nos] cassette in pLM 319
ggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaagatcttctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagcccgggggcgcgccatgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagaccttcaaacaccaagctttcaacaaactggctagcctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataacctgcaggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 7 - DNA, 32 bp, Artificial sequence
  Fwd PCR primer for amplification of I-SceI with AscI restriction site upstream of initiating ATG
aggcgcgccatgaaaaacatcaaaaaaaacca

SEQ ID NO: 8 - DNA, 29 bp, Artificial sequence
  Rev PCR primer for amplification of I-SceI with SbfI site downstream of TAA termination codon
gctcctgcaggttatttcaggaaagtttc

SEQ ID NO: 9 - DNA, 22 bp, Artificial sequence
  Oligonucleotide used with SeqID #10 to generate an I-SceI recognition site flanked by SalI and XbaI sites
tcgatagggataacagggtaat

SEQ ID NO: 10 - DNA, 22 bp, Artificial sequence
  Oligonucleotide used with SeqID #9 to generate an I-SceI recognition site flanked by SalI and XbaI sites
ctagattaccctgttatcccta

SEQ ID NO: 11 - DNA, 18 bp, Artificial sequence
  Oligonucleotide used with SeqID #12 to generate an I-SceI recognition site flanked by PacI sites
tagggataacagggtaat

SEQ ID NO: 12 - DNA, 18 bp, Artificial sequence
  Oligonucleotide used with SeqID #11 to generate an I-SceI recognition site flanked by PacI sites
taccctgttatccctaat

SEQ ID NO: 13 - DNA, 18 bp, Artificial sequence
  Fwd PCR primer used for RT-PCR analysis of maize endogenous constitutively expressed gene used as internal control for transgene expression level assays
tctgccttgcccttgctt

SEQ ID NO: 14 - DNA, 23 bp, Artificial sequence
  Rev PCR primer used for RT-PCR analysis of maize endogenous constitutively expressed gene used as internal control for transgene expression level assays
caattgcttggcaggtcttattt

SEQ ID NO: 15 - DNA, 20 bp, Artificial sequence
  Fwd PCR primer used for RT-PCR analysis of transgene expression levels via amplification of 3′ UTR region encoded by NOS terminator
tccccgatcgttcaaacatt

SEQ ID NO: 16 - DNA, 25 bp, Artificial sequence
  Rev PCR primer used for RT-PCR analysis of transgene expression levels via amplification of 3′ UTR region encoded by NOS terminator
ccatctcataaataacgtcatgcat

SEQ ID NO: 17 - DNA, 21 bp, Artificial sequence
  Fwd PCR primer used for RT-PCR analysis of GUS expression levels
ttacgtggcaaaggattcgat

SEQ ID NO: 18 - DNA, 20 bp, Artificial sequence
  Rev PCR primer used for RT-PCR analysis of GUS expression levels
gccccaatccagtccattaa

SEQ ID NO: 19 - DNA, 23 bp, Artificial sequence
  Fwd primer for RT-PCR analysis of expression of I-SceI
gaccaggtatgtctgctgtacga

SEQ ID NO: 20 - DNA, 24 bp, Artificial sequence
  Rev primer for RT-PCR analysis of expression of I-SceI
caggtggttaacacgttctttttt

SEQ ID NO: 21 - DNA, 23 bp, Artificial sequence
  Fwd PCR primer used for detection of homologous recombination of JB034 locus
gatcgcagtgcgtgtgtgacacc

SEQ ID NO: 22 - DNA, 21 bp, Artificial sequence
  Rev PCR primer used for detection of homologous recombination of JB034 locus
gtccgcatcttcatgacgacc

SEQ ID NO: 23 - DNA, 21 bp, Artificial sequence
  Fwd PCR primer used for detection of homologous recombination of JB039 locus
ctaatggtggggctttcaagg

SEQ ID NO: 24 - DNA, 23 bp, Artificial sequence
  Rev PCR primer used for detection of homologous recombination of JB039 locus.  When used with SeqID #23, amplification of a recombined locus should yeild a 0.9Kb product, while amplification of a native locus should amplify a 6.7Kb p
ccttaaggcgatcgcgctgaggc

SEQ ID NO: 25 - DNA, 20 bp, Artificial sequence
  Rev PCR primer used for confirming the presence of the JB039 locus. When used with SeqID #23, amplification of an unrecombined locus should yeild a 1.2Kb product, while the recombined locus should fail to produce a PCR product in this
agtgtacggaataaaagtcc

SEQ ID NO: 26 - DNA, 708 bp, Artificial sequence
  I-SceI gene codon optimized for expression in maize and soybean using the Leto program from Entelechon GmbH, Regensburg, Germany.
atgaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggcttctctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtga

SEQ ID NO: 27 - DNA, 923 bp, Artificial sequence
  Optimized Coding region of the Enzyme with Attachment regions
caaataatgattttattttgactgatagtgacctgttcgttgcaacaaattgatgagcaatgcttttttataatgccaactttgtacaaaaaagcaggggcgcgccaccatgaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggcttctctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtgacctgcaggccagctttcttgtacaaagttggcattataagaaagcattgcttatcaatttgttgcaacgaacaggtcactatcagtcaaaataaaatcattatttg

SEQ ID NO: 28 - DNA, 30 bp, Artificial sequence
  Recognition sequence of I-SceI
agttacgctagggataacagggtaatatag

SEQ ID NO: 29 - DNA, 18 bp, Artificial sequence
  Core sequence of I-SceI
tagggataacagggtaat